(19) United States
(12) Patent Application Publication
(10) Pub. No.: US 2026/0062678 A1
(43) Pub. Date: Mar. 5, 2026

(54) CELL REPLACEMENT THERAPY FOR PULMONARY DISEASES

(71) Applicant: The Trustees of Columbia University in the City of New York, New York, NY (US)

(72) Inventors: Hans-Willem Snoeck, New York, NY (US); Hsiao-Yun LIU, New York, NY (US)

(73) Assignee: The Trustees of Columbia University in the City of New York, New York, NY (US)

(21) Appl. No.: 19/381,083

(22) Filed: Nov. 6, 2025

Related U.S. Application Data
(63) Continuation of application No. PCT/US2024/028885, filed on May 10, 2024.
(60) Provisional application No. 63/501,453, filed on May 11, 2023.
(60) Provisional application No. 63/517,464, filed on Aug. 3, 2023.
(60) Provisional application No. 63/612,038, filed on Dec. 19, 2023.

(51) Int. Cl. C12N 5/071; A61K 35/42; A61P 11/00

(52) U.S. Cl. CPC C12N 5/0689; A61K 35/42; A61P 11/00; C12N 2500/38; C12N 2501/01; C12N 2501/11; C12N 2501/117; C12N 2501/119; C12N 2501/155; C12N 2501/33; C12N 2501/39; C12N 2501/727; C12N 2502/1323; C12N 2506/45

(57) ABSTRACT

Disclosed are lung progenitor cells and methods of generating the lung progenitor cells starting from mammalian pluripotent stem cells. The lung progenitors can be used as therapeutic treatments for various pulmonary disorders or injuries. The lung progenitor cells may also be used to model lung diseases/conditions and screen for desired agents.

### CROSS-REFERENCE TO RELATED APPLICATIONS

The present application is a continuation of International Patent Application No. PCT/US2024/028885 filed May 10, 2024, which claims priority to U.S. Provisional Patent Application Nos. 63/501,453 (filed on May 11, 2023), 63/517,464 (filed on Aug. 3, 2023), and 63/612,038 (filed on Dec. 19, 2023), each of which is incorporated herein by reference in its entirety.

### STATEMENT OF GOVERNMENT SUPPORT

This invention was made with government support under HL174927, HL142727, and HL134760 awarded by the National Institutes of Health. The government has certain rights in the invention.

### FIELD OF THE INVENTION

The present invention relates to methods of generating lung progenitor cells. The lung progenitors can be used as therapeutic treatments for various pulmonary disorders or injuries. The lung progenitor cells may be used to model lung diseases/conditions and screen for desired agents.

### BACKGROUND

Idiopathic pulmonary fibrosis (IPF) is a lethal fibrotic adult lung disease with a prevalence of ˜140,000 in the US and a survival of 3-4 years.^{1,2} Age, exposures and genetic predisposition^{3,4,5} are risk factors that compromise the function of alveolar epithelial cells, leading to aberrant, fibrotic injury repair. Childhood interstitial lung disease (chILD) is a related group of rare pediatric genetic diseases caused by mutations in genes involved in alveolar surfactant production and characterized by clinical manifestations ranging from neonatal respiratory failure and death to interstitial lung disease (ILD) and fibrosis later in life.^{6,7} The respiratory epithelium contains basal (BC), ciliated, secretory, goblet and neuroendocrine cells in the airways, and alveolar type 1 (AT1) and type 2 alveolar epithelial (AT2) cells in the alveoli, where gas exchange takes place. Critical to pathogenesis of both diseases is the AT2 cell which produce surfactants that prevent alveolar collapse. Mutations or variants in genes involved in surfactant production predispose to both IPF and chILD. Furthermore, mutations affecting telomere integrity are associated with IPF.^{1,2} Two drugs slow progression of IPF but neither is curative.⁸⁻¹⁰ Although lung transplantation is the sole life-saving treatment for IPF and severe chILD, the 5-year survival rate of recipients is only 59%, while the morbidity is incapacitating.¹¹ While genetic therapies have been proposed for chILD, vectors that specifically and efficiently target alveolar epithelial cells have not yet been identified.

Although hPSC-derived, eventually gene-corrected, AT2 cells would appear a logical choice for cell replacement therapy (CRT), the distal lung stem cell compartment needs to be at least partially replaced to achieve durable improvement. This idea is based on the fact that while a fraction of AT2 cells participate in alveolar maintenance in steady-state,¹⁸⁻²⁰ populations derived from distal airways play a major role after injury.²¹⁻²⁹ Importantly, some of these accumulate in IPF lungs.^{26,27,30,31} Modeling in organoids derived from human pluripotent stem cells (hPSCs) provided evidence that distal lung stem cell populations involved in regeneration in humans are functionally affected by genetic mutations associated with IPF and chILD as well.

While age and certain exposures are risk factors, up to 20% of IPF patients show familial predisposition.^{2,42} Genetic studies revealed two classes of molecularly distinct etiologies that underly susceptibility to IPF: mutation or variants in genes that maintain mitotic chromosome and telomere integrity,^{3,4} and in genes affecting surfactant production.⁵ The implication of mutations in surfactant proteins SFTPA⁴³⁻⁴⁶ and SFTPC^{47,48} suggest a key role of the surfactant-producing AT2 cells. Such mutations result in an unfolded protein response in the endoplasmic reticulum (UPR^{ER}), mistrafficking of surfactant proteins or impaired autophagy, suggesting proteotoxic stress as one pathogenetic mechanism.^{5,49-52} Some Hermansky-Pudlak Syndrome (HPS) mutations show a high incidence HPS-associated interstitial pneumonia (HPSIP), an entity similar to IPF.⁵³⁻⁵⁶ HPS is caused by abnormal biogenesis and trafficking of lysosome-related organelles (LROs) and characterized by pigmentation abnormalities and bleeding diathesis associated with dysfunction of melanosomes and platelet delta granules, respectively, which are, similar to lamellar bodies (LBs) of AT2 cells, LROs.⁵⁷⁻⁵⁹ Mutations in ABCA3, a phospholipid transporter in the limiting membrane of LBs cause a spectrum of clinical manifestations including neonatal respiratory distress and childhood ILD, but has been associated with IPF was well.⁶⁰⁻⁶⁴ 8 to 15% of patients with familial IPF have heterozygous mutations in telomerase components or genes involved in telomere maintenance.⁶⁵⁻⁷⁶ Furthermore, several susceptibility loci have been identified that affect telomere length.⁷⁷ Since DNA damage responses and proteostasis are connected^{78,79} and since senescence and DNA damage induce ER stress through as yet unknown mechanisms,⁸⁰ the pathogenesis of IPF may converge on failure to maintain cellular quality control in surfactant-producing AT2 cells, because they devote their endosomal and proteostatic machinery to homeostasis of highly hydrophobic surfactant proteins.⁵ Supporting the notion that an appropriate ER stress response is critical to prevent fibrosis, mice with the deletion of the ER stress sensor, Grp78 (aka BIP) develop spontaneous fibrosis.⁸¹ It is therefore possible that fibrosis is not the result of enhanced UPR^{ER} per Se, but a consequence of a defective downstream response to increased ER stress in AT2 cells.

Abnormal differentiation may be another hallmark of IPF. Remodeled regions of IPF lungs express proximal markers (bronchiolization).⁸² An “aberrant basaloid cell” (aBC) population, expressing KRT17, COL1A1 and other ECM components and lacking BC markers such as KRT5, is present but rare in normal lungs³⁰ (“aberrant” is therefore a misnomer), but abundant in fibrotic lesions.^{30,31} In mice, pre-AT1 transitional state (PATS) cells have been described that are transitional between AT2 and AT1 fates, accumulate after bleomycin injury and share expression signatures with human aBCs (and are therefore called PATS-like in humans).⁸³ A population of KRT8+“alveolar differentiation intermediate” (ADI) cells with characteristics similar to PATS cells and to human aBCs has been identified as well.²³ Both PATS-like and ADI cells are increased in fibrotic lesions in IPF,^{23,83} and regions with abundant presence of PATS-like cells lacked expression RAGE⁺ AT1 cells.⁸³ These findings are suggestive of a differentiation defect from the AT2 towards the AT1 lineage in IPF.⁸³ Further supporting this notion is the finding that prevention of AT1 differentiation after pneumonectomy resulted in fibrosis, whereas deletion of AT1 is sufficient to cause spontaneous fibrosis in mice.⁸⁴ Furthermore, several types of putative alveolar progenitor cells, collectively called transitional cells, or respiratory airway/terminal respiratory bronchiole secretory (RA/TRB) cells and characterized by expression of the secretory cell marker, SCGB3A2, reside in terminal bronchioles of humans and are more prevalent in fibrotic lesions in IPF lungs.^{26,27,30} SCGB3A2 also marks a transitional AT2-like cell, the AT0 cell.²⁶ The exact lineage relations and overlap among these cell types is unclear, but they are mostly likely at least in part transitional between AT2 and AT1 cells, can be direct AT2 precursors, and may be derived from distal airway progenitors or secretory cells. While the accumulation of these populations may be viewed as reactive to AT2 injury, we show here that they may be intrinsically dysfunctional and also play a causative role in IPF.

Regenerative medicine holds promise for new treatment options. Diseases that are amenable to cellular therapies encompass both airway and distal lung disease. Replacing damaged lung tissue with stem cell-derived cells may provide improvement or even cure. Novel approaches for cell replacement therapy for lung diseases are urgently needed.

### SUMMARY

The present disclosure provides for a method for generating lung progenitor cells. The method may comprise: (a) producing anterior foregut endoderm cells from mammalian pluripotent stem cells (PSCs); (b) culturing the anterior foregut endoderm cells in a suspension culture comprising a glycogen synthase kinase (GSK) inhibitor, a bone morphogenic protein (BMP) agonist, at least one fibroblast growth factors (FGF) agonist, and retinoic acid, to generate a lung bud organoid (LBO); (c) dissociating the LBO and culturing the dissociated LBO on a first cell culture substrate in presence of a GSK inhibitor, a BMP agonist, at least one FGF agonist, and retinoic acid, to generate plated LBO, wherein the first cell culture substrate is coated with a first biomolecule; and (d) culturing the plated LBO on a second cell culture substrate in a first culture medium or a second culture medium, wherein the first culture medium comprises an inhibitor of Rho kinase (ROCK), a GSK inhibitor, at least one FGF agonist, a BMP agonist, and retinoic acid, wherein the second culture medium comprises insulin, an epidermal growth factor (EGF) agonist, a corticosteroid, a 3′,5′-cyclic adenosine monophosphate (cAMP) pathway activator, and an inhibitor of ROCK, wherein the second cell culture substrate is coated with a second biomolecule.

The GSK inhibitor may be CHIR99021. The GSK inhibitor may be at a concentration ranging from about 1 μM to about 10 μM, or at a concentration of about 3 μM.

The BMP agonist may be BMP4. The BMP agonist may be at a concentration ranging from about 5 ng/ml to about 20 ng/ml, or at a concentration of about 10 ng/ml.

The at least one FGF agonist may comprise FGF10 and keratinocyte growth factor (KGF). The at least one FGF agonist may be at a concentration ranging from about 5 ng/ml to about 20 ng/ml, or at a concentration of about 10 ng/ml.

Retinoic acid may be at a concentration ranging from about 20 nM to about 80 nM, or at a concentration of about 50 nM.

In certain embodiments, the BMP agonist is BMP4, the at least one FGF agonist is KGF and FGF10, where KGF, FGF10, and/or BMP4 are each at a concentration of about 10 ng/ml.

The inhibitor of ROCK may be Y27632. The inhibitor of ROCK may be at a concentration ranging from about 5 μM to about 15 μM, or at a concentration ranging from about 5 μM to about 10 μM. The inhibitor of ROCK may be at a concentration of about 10 μM in the first culture medium. The inhibitor of ROCK may be at a concentration of about 5 μM in the second culture medium.

Insulin may be at a concentration ranging from about 1 μg/ml to about 10 μg/ml, or at a concentration of about 5 μg/ml.

The EGF agonist may be EGF. The EGF agonist may be at a concentration ranging from about 0.05 ng/ml to about 0.5 ng/ml, or at a concentration of about 0.1 ng/ml.

The corticosteroid may be hydrocortisone. The corticosteroid may be at a concentration ranging from about 10 ng/ml to about 50 ng/ml, or at a concentration of about 25 ng/ml.

The cAMP pathway activator may be cholera toxin. The cAMP pathway activator may be at a concentration ranging from about 1 ng/ml to about 10 ng/ml, or at a concentration of about 8 ng/ml.

The first culture medium may be serum-free or may comprise serum. The first culture medium may comprise a serum substitute. The first culture medium may comprise a cell culture medium conditioned by feeder cells.

The second culture medium may be serum-free or may comprise serum. The second culture medium may comprise a serum substitute. The second culture medium may comprise a cell culture medium conditioned by feeder cells.

The feeder cells may be fibroblasts, such as 3T3-J2 cells.

The first biomolecule may comprise a solubilized basement membrane preparation from the Engelbreth-Holm-Swarm (EHS) mouse sarcoma. The first biomolecule may comprise gelatin and/or collagen.

The second biomolecule may comprise a solubilized basement membrane preparation from the Engelbreth-Holm-Swarm (EHS) mouse sarcoma. The second biomolecule may comprise gelatin and/or collagen.

The first cell culture substrate and the second cell culture substrate may be identical, or may be different. The first biomolecule and the second biomolecule may be identical, or may be different.

The LBO may comprise (i) lung epithelial cells expressing FOXA2, FOXA1, NKX2.1 and EPCAM, and (ii) mesenchymal progenitors expressing PDGFRa, CD90, TBX4 and HOXA5.

In step (c), the LBO may be dissociated to single cells.

The mammalian pluripotent stem cells (PSCs) may be human pluripotent stem cells (hPSCs). The mammalian pluripotent stem cells (PSCs) may be embryonic stem cells (ESCs) and/or induced pluripotent stem cells (iPSCs).

The lung progenitor cells may comprise secretory-like cells, and airway basal-like cells. The lung progenitor cells may further comprise fibroblasts, T cells and dendritic cells.

The lung progenitor cells may comprise (i) cells expressing MUC1, NOTCH3, UPK3A, KRT4, and KRT13, and (ii) cells expressing p63, KRT5, KRT17, ITGB4, JAG2 and DLK2.

The lung progenitor cells may comprise MUC1^{hi}CD104^{lo} cells and MUC1^{lo} CD104^{hi} cells.

The present disclosure also provides for lung progenitor cells generated by the present methods.

The present disclosure provides for a cell population comprising the present lung progenitor cells.

Also provided in the present disclosure is a cell population comprising lung progenitor cells generated by the present methods.

The present disclosure provides for lung progenitor cells generated in vitro, comprising secretory-like cells, and airway basal-like cells. The lung progenitor cells may further comprise fibroblasts, T cells and dendritic cells.

The present disclosure provides for lung progenitor cells generated in vitro, comprising cells expressing MUC1, NOTCH3, UPK3A, KRT4, and KRT13, and cells expressing p63, KRT5, KRT17, ITGB4, JAG2 and DLK2.

The present disclosure provides for a pharmaceutical composition comprising the present lung progenitor cells, or the present cell population.

The present disclosure provides for a method of treating a pulmonary disorder or injury in a subject in need thereof. The method may comprise administering to the subject an effective amount of the present lung progenitor cells, or the cell population. The method may comprise administering to the subject the present pharmaceutical composition.

The pulmonary disorder or injury may be: cystic fibrosis; emphysema; chronic obstructive pulmonary disease (COPD); pulmonary fibrosis; idiopathic pulmonary fibrosis (IPF); Hermansky-Pudlak Syndrome; hypersensitivity pneumonitis; sarcoidosis; asbestosis; autoimmune-mediated interstitial lung disease; pulmonary hypertension; lung cancer; acute lung injury (adult respiratory distress syndrome); respiratory distress syndrome of prematurity, chronic lung disease of prematurity (bronchopulmonary dysplasia); surfactant protein B deficiency, surfactant protein C deficiency, ABCA3 deficiency; NKX2.1 mutation; ciliopathies; congenital diaphragmatic hernia; pulmonary alveolar proteinosis; pulmonary hypoplasia; lung injury, and combinations thereof.

The pulmonary disorder or injury may be an interstitial lung disease or a congenital surfactant deficiency.

The lung progenitor cells may be non-syngeneic, or syngeneic, with the subject.

The lung progenitor cells may be allogeneic or xenogeneic with the subject.

### BRIEF DESCRIPTION OF THE DRAWINGS

FIGS. 1A-1G. Characterization of iRAPs. FIG. 1A: Schematic of experimental design. FIG. 1B: Representative images of iRAPs generated from iPSCs. FIG. 1C: Cell expansion of iPSC-derived iRAPs. FIG. 1D: Representative confocal images of expression of indicated markers between 1 and 6 passages of ESC-derived iRAPs. FIG. 1E: Transmission electron microscopy (TEM). FIG. 1F: UMAP feature plots of scRNAseq data from ESC-derived iRAPs for indicated markers. FIG. 1G: Comparative cell identity assignment of scRNAseq data of the distal respiratory airways of human lung as reported by Murthy et al. (Nature 2022) (left) and of iRAPs (right).

FIGS. 2A-2F. Characterization of D-iRAPs and AT0/AT2 cells. FIG. 2A: Schematic of experimental design. FIG. 2B: Flow cytometric analysis of HT1-56 and HT2-280 in iRAPs and D-iRAPs, with quantification (bottom) *p<0.05, **p<0.01, ****p<0.0001, one-way ANOVA with Tukey multiple comparison test (n=3). FIG. 2C: Z-stack confocal 3D images. Scale bars=20 μm. FIG. 2D: Cell identity assignment of D-iRAP scRNA-seq dataset based on HLCA. FIG. 2E: UMAP feature plots of indicated markers. FIG. 2F: Expression of SFTPC mRNA in HT2-280- and HT2-280⁺ cells isolated from D-iRAPs (n=3, one-way ANOVA).

FIGS. 3A-3J. Generation of iAT1 cells. FIG. 3A: Schematic of experimental design. FIG. 3B: Representative immunofluorescence (IF) images for indicated markers in DCI-SB (“Ctrl-ALI”) and BMP-SB 2D conditions. Scale bars=50 μm. FIG. 3C: RT-qPCR comparing different conditions (one-way ANOVA with multiple comparison using Tukey test, n>4). FIG. 3D: Representative flow cytometry plot for HT1-56 in iAT1 cells (left) with quantification of HT1-56 and HT2-280 (n=3) throughout differentiation (right). FIG. 3E: IF for YAP, zonula occludens (ZO)-1 and RAGE at different plating densities in iAT1 cells. FIGS. 3F-3H: Quantitative analysis of amount of RAGE/cell (FIG. 3F), cell size a determined by ZO-1 outline (FIG. 3G), and the fraction of YAP+ nuclei (FIG. 3H) at different cell densities.

FIG. 3I: Fraction of HT1-56 (AT1-committed) and HT-280+(AT2-committed) cells in D-iRAPs generated in the presence of LATSi. FIG. 3J: Cell identity assignment of iAT1 scRNA-seq dataset and UMAP feature plots of indicated markers.

FIGS. 4A-4G. Phenotype of HSP1^{-/-} iRAPs. FIG. 4A: TEM showing that multivesicular bodies containing “onion-ring” structures are present in WT iRAPs that appear empty in HSP1^{-/-} iRAPs. FIG. 4B: Representative images of Lysotracker® uptake in iRAPs (scale bar=250 μm). FIG. 4C: Representative flow cytometry histogram of Lysotracker© uptake in WT and HSP1^{-/-} iRAPs, with quantification (right). ***p<0.001, One-way ANOVA with Dunnett's multiple comparison test, n=3. FIG. 4D: UMAP feature plots of indicated markers in WT and HPS^{-/-} iRAPs. FIGS. 4E-4F: mRNA expression for indicated markers in WT and HPS1^{-/-} iRAPs. *p<0.05, **p<0.01, Student's unpaired t-test, WT n=6, HPS1^{-/-} n=3. FIG. 4G: Expression of KRT17 (KRT5 was absent) in tile scans of multiple WT and HSP1−/− iRAPs with quantification on the right.

FIG. 5. ER stress in HPS1−/− iRAPs. Left and middle panels: mRNA expression for indicated markers after 5 hours of 5 ng/mL tunicamycin exposure. *p<0.05, **p<0.01, Student's unpaired t-test, n=3. Right panel: Apoptosis after 5 days of 0.1 mg/mL tunicamycin treatment normalized to untreated iRAPs of the same genotype. *p<0.05, Student's unpaired t-test, n>4.

FIGS. 6A-6N. Defects in HSP1^{-/-} D-iRAPs and iAT1 cells. FIG. 6A: Differentiation schematic (D-iRAP highlighted). FIG. 6B: Representative flow cytometry plot for HT1-56. FIG. 6C: quantification of HT1-56⁺ cells in HSP1^{-/-} D-iRAPs normalized to WT. **p<0.01, Student's unpaired t-test, n=11. FIG. 6D: mRNA expression for AGER mRNA in HPS-1^{-/-} and WT D-iRAPs. **p<0.01, Student's t-test, n=3. FIG. 4E: Representative flow cytometry contour plot of HT2-280 expression in WT and HPS1^{-/-} D-iRAPs. FIG. 6F: quantification of HT2-280⁺ cells in HSP1^{-/-} D-iRAPs normalized to WT. *p<0.05, Student's unpaired t-test, n>10. FIG. 6G: mRNA expression for indicated markers in WT and HPS1^{-/-} D-iRAPs compared to iRAPs of the same genotype. ***p<0.001, Two-way ANOVA with Tukey's multiple comparison, n=3. FIG. 6H: Differentiation schematic (iAT1 highlighted). FIG. 6I: Representative flow cytometry plots of HT1-56 in WT and HPS1^{-/-} iAT1 cells. FIG. 6J: Quantification of FIG. 41. FIG. 6K: mRNA expression for AGER mRNA in HPS1^{-/-} and WT D-iRAPs. **p<0.01, Student's t-test, n=3. FIG. 6L: Expression of RAGE in tile scans of WT and HSP1−/− iAT1 cells with quantification on the right, *p<0.05, Student's t-test, n=3. FIG. 6M: Expression of nuclear YAP in tile scans of WT and HSP1−/− iAT1 cells with quantification on the right, **p<0.01, Student's t-test, n=3. FIG. 6N: Effect of tunicamycin on expression of HT1-56 (AT1) and HT2-280 (AT2) expression in WT D-iRAPs, **p<0.01, Student's t-test, n=3.

FIG. 7. iRAPs conclusion. Schematic overview of the identity and potential of iRAPs as well as defects observed with mutations associated with familial IPF or iRAPs exposed to ER stress.

FIGS. 8A-8F. Characterization of DLEPs. FIG. 8A: Generation of DLEPs from 3D organoids (represented by light sheet microscopic image). FIG. 8B: Expansion of DLEPs generated from an ESC and an iPSC line. FIG. 8C: Cell identity assignment of DLEPs based on the Human Lung Cell Atlas and representative feature plots identifying BC-like, secretory-like (variant secretory) cells and fibroblastic cells. FIG. 8D: IF for p63, KRT5 and KRT17 in ESC-derived DLEPs. FIG. 8E: Representative feature plots comparing DLEPs and the trachea-engrafting iBC recently reported by Ma et al. (Cell Stem Cell 2023). FIG. 8F: Flow cytometric analysis of CD104 and MUC1 expression in ESC- and iPSC-derived DLEPs cultured with and without DAPT.

FIGS. 9A-9D. Regional de-epithelialization. FIG. 9A: Schematic representation and IF for ProSPC (AT2), AQ5 (AT1), and CD31 (endothelial cells). De-epithelialized area outlined by dotted white lines. Representative squares shown at higher magnification (lower panels). FIG. 9B: IF for epithelial cells (AQ5, ProSPC), and endothelial cells (CD31) in treated region 5 and 10 days post de-epithelialization. FIG. 9C: LIS of de-epithelialized region after 2, 5, and 10 days (UN=untreated). Mean±SEM, one-way ANOVA, n=3 animals per condition, 60 ROI (30 from left lung, 30 from right lung) per animal; *, p=0.0287; ****, p<0.0001. FIG. 9D: EdU incorporation after de-epithelialization with and without irradiation.

FIGS. 10A-10E. Engraftment of DLEPs. FIG. 10A: Schematic representation of experimental approach. FIG. 10B: Annotations (white squares) of clusters of human cells based on hMit staining, using de-epithelialized, non-engrafted lung as background control. FIG. 10C: RNA in situ hybridization (RNAscope) for human b2-microglobulin (hB2M), and IF for hMit, hKRT5, hTP63, ProSPB, ProSPC, RAGE and SCGB3A2 in representative engrafted areas from left lung. (White arrowheads: human cells co-expressing hMit with ProSPC). The left upper panel shows a region without human cells as a negative control for RNAscope. FIG. 10D: Percent engraftment in lower left lung measured by flow cytometry for B2M. FIG. 10E: Percent engraftment in the most highly engrafted for each experiment. DE=definitive endoderm negative control. Mean±SEM, unpaired Student's t-test (n=6 per condition; *, p=0.02). Pooled equal numbers of experiments with ESC- and iPSC-derived DLEPs.

FIG. 11. Expression of SPC in engrafted DLEPs in rats conditioned with de-epithelialization and irradiation on day 10. Representative areas showing engraftment of human cells based on hMit staining in different rats conditioned with de-epithelialization and irradiation (Arrowheads: examples of human cells co-expressing hMit and RAGE; human cells co-expressing hMit and SPC; native rat SPC⁺ cells). Single channels and a representative higher magnification area of engrafted cells are highlighted in the white square.

FIGS. 12A-12B. Injury repair. FIG. 12A: H&E staining of injured (De-epi+IR) and engrafted (De-Epi+IR+DLEPs) lower left lobes. Lower panels: Representative higher magnification images. FIG. 12B: Lung injury scores (IR: irradiation; LLL: lower left lung; RL: right lung, UN: control lung, 800 blindly evaluated fields in total, 30 ROI per tile scan, one-way ANOVA; *, p=0.0383; ****, p<0.0001).

FIGS. 13A-13C. Culture conditions FIG. 13A: Schematic overview of the protocol to generate LOs. FIG. 13B: Expansion of DLEPs generated under indicated conditions. FIG. 13C: Flow cytometric profile of DLEPs generated under indicated conditions.

### DETAILED DESCRIPTION

The present disclosure provides for lung progenitor cells and methods of generating the lung progenitor cells. These lung progenitor cells may be used to engraft the lungs, serving as a cell replacement therapy or a regenerative therapy for treating various lung diseases, conditions, and injuries. The lung progenitor cells may be prepared from cells of subjects with mutation(s) and subsequently used to define relevant factor(s) associated with the mutation(s).

Cell replacement therapy (CRT), involving exchange of diseased cells with healthy cells to halt or reverse the disease, holds the promise of providing a curative and equitable treatment for pulmonary disorders or injuries such as chILD and IPF. However, progress has been hindered by the inability to identify appropriate engrafting cells and the lack of human-sized animal models.

CRT needs an unlimited supply of cells capable of engrafting and/or of promoting lung repair. A drug screening platform requires expandable cells that faithfully recapitulate defects associated with the disease. In certain embodiments, the present method targets the distal lung stem cell compartment for efficacious CRT for pulmonary disorders or injuries such as IPF and chILD. In certain embodiments, the present distal lung stem cells constitute a cell-based platform for drug screening.

In certain embodiments, the present lung progenitor cells are derived from hPSCs. hPSCs undergo unlimited expansion in vitro, can be generated in a patient-specific or immunologically tolerated fashion,³² and are amenable to gene editing.

The present disclosure provides for a method for generating lung progenitor cells. The method may comprise: (a) producing anterior foregut endoderm cells from mammalian pluripotent stem cells (PSCs); (b) culturing the anterior foregut endoderm cells in a suspension culture comprising a glycogen synthase kinase (GSK) inhibitor, a bone morphogenic protein (BMP) agonist, one or more FGF agonists, and retinoic acid, to generate a (or at least one) lung bud organoid (LBO); (c) dissociating the LBO and culturing the dissociated LBO on a first cell culture substrate in the presence of a GSK inhibitor, a BMP agonist, one or more FGF agonists, and retinoic acid, to generate plated LBO, wherein the first cell culture substrate is coated with a first biomolecule; and (d) culturing the plated LBO on a second cell culture substrate in a first culture medium or a second culture medium, wherein the first culture medium comprises an inhibitor of Rho kinase (ROCK), a GSK inhibitor, at least one FGF agonist, a BMP agonist, and retinoic acid, wherein the second culture medium comprises insulin, an agonist of the EGF signaling pathway, a corticosteroid, a 3′,5′-cyclic adenosine monophosphate (cAMP) pathway activator, and an inhibitor of ROCK, wherein the second cell culture substrate is coated with a second biomolecule.

In certain embodiments, the present method for generating lung progenitor cells may comprise: (a) producing anterior foregut endoderm cells from mammalian stem cells (e.g., mammalian pluripotent stem cells (PSCs)); (b) culturing the anterior foregut endoderm cells in a suspension culture comprising a Wnt agonist (e.g., a glycogen synthase kinase (GSK) inhibitor such as CHIR99021, etc.), a bone morphogenic protein (BMP) agonist (e.g., BMP4), one or more FGF agonists (e.g., FGF10, keratinocyte growth factor (KGF)), and retinoic acid (or its derivative), to generate a (or at least one) lung bud organoid (LBO); (c) dissociating the LBO and culturing the dissociated LBO on a first cell culture substrate in the presence of a Wnt agonist (e.g., a GSK inhibitor such as CHIR99021), a BMP agonist (e.g., BMP4), one or more FGF agonists (e.g., FGF10, KGF), and retinoic acid (or its derivative), to generate plated LBO; and (d) culturing the plated LBO on a second cell culture substrate in a first culture medium or a second culture medium, wherein the first culture medium comprises an inhibitor of Rho kinase (ROCK) (e.g., Y27632), a Wnt agonist (e.g., a GSK inhibitor such as CHIR99021), one or more FGF agonists (e.g., FGF10, KGF), a BMP agonist (e.g., BMP4), and retinoic acid (or its derivative), wherein the second culture medium comprises insulin, an agonist of the EGF signaling pathway (e.g., EGF), a corticosteroid (e.g., hydrocortisone), a 3′,5′-cyclic adenosine monophosphate (cAMP) pathway activator (e.g., cholera toxin), and an inhibitor of ROCK (e.g., Y27632), wherein the second cell culture substrate is coated with a second biomolecule.

In certain embodiments, the LBO may be dissociated to single cells or cell clusters.

In certain embodiments, the first culture medium may comprise one or more of Y27632, CHIR99021, FGF10, KGF, BMP4, and retinoic acid. In one embodiment, Y27632 is at a concentration of about 0.5 μM to about 20 μM, about 1 μM to about 15 μM, about 1 μM to about 10 μM, about 5 μM to about 10 μM, about 5 μM, or about 10 μM. In one embodiment, CHIR99021 is at a concentration of about 0.5 μM to about 10 μM, about 1 μM to about 8 μM, about 1 μM to about 5 μM, or about 3 μM. In one embodiment, KGF is at a concentration of about 1 ng/ml to about 50 ng/ml, about 2 ng/ml to about 30 ng/ml, about 5 ng/ml to about 15 ng/ml, or about 10 ng/ml. In one embodiment, FGF10 is at a concentration of about 1 ng/ml to about 50 ng/ml, about 2 ng/ml to about 30 ng/ml, about 5 ng/ml to about 15 ng/ml, or about 10 ng/ml. In one embodiment, BMP4 is at a concentration of about 1 ng/ml to about 50 ng/ml, about 2 ng/ml to about 30 ng/ml, about 5 ng/ml to about 15 ng/ml, or about 10 ng/ml. In one embodiment, retinoic acid is at a concentration of about 10 nM to about 100 nM, about 20 nM to about 80 nM, about 30 nM to about 60 nM, about 40 nM to about 70 nM, about 40 nM, or about 50 nM.

In certain embodiments, the second culture medium may comprise one or more of insulin, EGF, hydrocortisone, cholera toxin, and Y27632. In one embodiment, insulin is at a concentration of about 1 μg/ml to about 50 μg/ml, about 2 μg/ml to about 30 μg/ml, about 5 μg/ml to about 15 μg/ml, or about 5 μg/ml. In one embodiment, EGF is at a concentration of about 0.01 ng/ml to about 1 ng/ml, about 0.05 ng/ml to about 0.8 ng/ml, about 0.05 ng/ml to about 0.5 ng/ml, about 0.05 ng/ml to about 0.2 ng/ml, or about 0.1 ng/ml. In one embodiment, hydrocortisone is at a concentration of about 1 ng/ml to about 50 ng/ml, about 2 ng/ml to about 40 ng/ml, about 5 ng/ml to about 30 ng/ml, about 10 ng/ml to about 30 ng/ml, or about 25 ng/ml. In one embodiment, cholera toxin is at a concentration of about 1 ng/ml to about 30 ng/ml, about 2 ng/ml to about 20 ng/ml, about 5 ng/ml to about 15 ng/ml, about 5 ng/ml to about 10 ng/ml, or about 8 ng/ml. In one embodiment, Y27632 is at a concentration of about 0.5 μM to about 20 μM, about 1 μM to about 15 μM, about 1 μM to about 10 μM, about 5 μM to about 10 μM, about 10 μM, or about 5 μM.

As used herein, the terms “first culture medium” and “first cell culture medium” are used interchangeably; the terms “second culture medium” and “second cell culture medium” are used interchangeably.

The cell culture medium (e.g., the first culture medium, or the second culture medium) may contain salts, amino acids, vitamins, minerals, trace metals, sugars, lipids, and/or nucleosides. By way of example and without limitation, the following cell culture media may be used as the basal cell culture medium: Dulbecco's Modified Eagle's Medium (DMEM), DMEM/F-12 (ThermoFisher), Essential 8™ Medium (ThermoFisher), TeSR™ (StemCell Technologies), StemPro™-34 (ThermoFisher), StemSpan™ (StemCell Technologies), Basal Medium Eagles (BME), Minimum Essential Medium (MEM), Ham's F12, RPMI 1640, Iscove's, McCoy's, Nutrient Mixture F-10 (HAM's F-10) and Nutrient Mixture F-12 (HAM's F-12). The culture media mentioned above are cited as illustrative examples of culture medium suitable for use in the present methods.

The first culture medium, or the second culture medium, may be serum-free. The first culture medium, or the second culture medium, may contain serum (for example fetal bovine serum).

When the cell culture medium (e.g., the first culture medium, or the second culture medium) is serum-free, it may contain a serum substitute.

Examples of serum substitutes include, but are not limited to, commercially available serum substitutes such as KnockOut™ Serum Replacement (ThermoFisher), Nutridoma (Boehringer Mannheim), B-27™ Supplement (ThermoFisher), BIT9500 (StemCell Technologies), X-VIVO-10 or X-VIVO-15 (Cambrex BioSciences), N2 supplement, Chemically-defined Lipid Concentrated (ThermoFisher), Glutamax (ThermoFisher), Ultroser™ (Sigma), and milk or milk fractions (such as nonfat dry milk filtrate).

In other embodiments, the serum substitute may contain one or more of the following: albumin (e.g., lipid-rich albumin, human serum albumin, bovine serum albumin etc.), transferrin, insulin, fatty acids, collagen precursors, collagen precursor, trace elements (e.g., zinc and selenium), 2-mercaptoethanol, 3′ thiol glycerol, or their equivalents and the like.

In certain embodiments, the first culture medium or the second culture medium may comprise a cell culture medium conditioned by feeder cells.

A conditioned medium can include a medium in which feeder cells have been grown.

The cell culture medium may be conditioned by cells grown in two-dimensional culture (e.g., a monolayer), or in three-dimensional culture.

Before collecting the conditioned medium, feeder cells may be cultured for a time sufficient to allow their adhesion to the culture substrate, their multiplication and the secretion of the components which characterize the conditioned medium. For example, feeder cells may be cultured for at least or about 3 hours, at least or about 12 hours, at least or about 24 hours, at least or about 48 hours, at least or about 72 hours, at least or about 4 days, at least or about 5 days, at least or about 6 days, at least or about 7 days, at least or about 8 days, at least or about 9 days, at least or about 10 days, at least or about 11 days, at least or about 12 days, at least or about 13 days, at least or about 14 days, at least or about 15 days, or even longer.

The conditioned medium may be prepared as follows: (i) culturing feeder cells in a cell culture medium (e.g., the first cell culture medium, or the second cell culture medium) for a time period as described herein; and (ii) separating the cell fraction from the liquid culture medium, thereby obtaining a conditioned medium.

In order to separate the cellular fraction from the medium, different methods may be used. For example, the conditioned medium can be processed by filtration using filters of adequate porosity. Alternatively, the separation of the conditioned medium can be achieved by centrifugation followed by cell sedimentation. In certain embodiments, the step of separation of the conditioned medium from the cellular component is performed by filtration or centrifugation, or a combination of both.

As used herein, feeder cells are intended to mean supporting cell types used alone or in combination. The cell type may further be of human or other species (e.g., mouse) origin. The tissue from which the feeder cells may be derived include embryonic, fetal, neonatal, juvenile or adult tissue, and it further includes tissue derived from skin, including foreskin, umbilical cord, muscle, lung, epithelium, placenta, fallopian tube, glandula, stroma or breast. The feeder cells may be derived from cell types such as fibroblasts, fibrocytes, myocytes, keratinocytes, endothelial cells and epithelial cells. Examples of specific cell types that may be used for deriving feeder cells include embryonic fibroblasts, extraembryonic endodermal cells, extraembryonic mesoderm cells, fetal fibroblasts and/or fibrocytes, fetal muscle cells, fetal skin cells, fetal lung cells, fetal endothelial cells, fetal epithelial cells, umbilical cord mesenchymal cells, placental fibroblasts and/or fibrocytes, placental endothelial cells.

The feeder cells may be fibroblasts, such as human foreskin fibroblasts (hFF) or mouse embryonic fibroblast (MEF) cells (e.g., 3T3 cells). The feeder cells may be SNL76/7 cells, and/or 10T1/2 cells.

The feeder cells may comprise irradiated cells such as irradiated fibroblasts. These feeder cells may be used after exposure to radiation or treated with a cell division inhibitor (such as mitomycin C) to stop the cell division.

Anterior foregut endoderm cells are first generated from mammalian pluripotent stem cells (PSCs). In certain embodiments, PSCs (e.g., ESC or iPSC cells) are cultured in a cell culture medium containing Y-27632 and BMP4 (e.g., the primitive streak/embryoid body medium containing about 10 μM Y-27632, about 3 ng/ml BMP4) to allow embryoid body formation. Embryoid bodies are fed (e.g., every day) with fresh cell culture media containing Y27632, BMP4, FGF2 and Activin A (e.g., the endoderm induction medium containing about 10 μM Y-27632, about 0.5 ng/ml BMP4, about 2.5 ng/ml FGF2 and about 100 ng/ml Activin A). Cells are then cultured in a cell culture medium containing Noggin and SB431542 (e.g., the anteriorization medium 1 containing about 100 ng/ml Noggin and about 10 μM SB431542) for a period of time (e.g., about 24 hours), followed by being cultured in a cell culture medium containing SB431542 and IWP2 (e.g., the anteriorization medium 2 containing about 10 μM SB431542 and about 1 μM IWP2) for a period of time (e.g., about 24 hours).

Lung bud organoids (LBOs) can be generated from anterior foregut endoderm cells. In certain embodiments, anterior foregut endoderm cells are cultured in a suspension culture (e.g., a branching medium) containing CHIR99021, FGF10, KGF, BMP4 and retinoic acid (e.g., the ventralization/branching medium containing about 3 μM CHIR99021, about 10 ng/ml FGF10, about 10 ng/ml KGF, about 10 ng/ml BMP4 and about 50 nM all-trans retinoic acid) for a period of time (e.g., about 48 hours) to form three-dimensional clumps which then fold into lung bud organoids or LBOs (e.g., as early as day 10 to day 12, with day 0 being the start of culturing the PSCs). The cell culture medium (e.g., the branching medium) is changed (e.g., regularly such as every other day until day 20 to day 25).

The LBOs are dissociated and plated on a first cell culture substrate which is coated with a first biomolecule, and then cultured in the presence of CHIR99021, FGF10, KGF, BMP4 and retinoic acid (e.g., the branching medium containing about 3 μM CHIR99021, about 10 ng/ml FGF10, about 10 ng/ml KGF, about 10 ng/ml BMP4 and about 50 nM all-trans retinoic acid) to generate plated LBOs.

As used herein, day 0 is the start of culturing the stem cells (e.g., PSCs), unless specifically stated otherwise.

Lung progenitor cells can be generated from LBOs. The plated LBOs are then cultured on a second cell culture substrate in a first culture medium or a second culture medium, where the second cell culture substrate is coated with a second biomolecule. In certain embodiments, the first culture medium may comprise an inhibitor of Rho kinase (ROCK) (e.g., Y27632), a Wnt agonist (e.g., a GSK inhibitor such as CHIR99021), one or more FGF agonists (e.g., FGF10, KGF), a BMP agonist (e.g., BMP4), and retinoic acid (or its derivative) (e.g., about 10 μM Y27632, about 3 μM CHIR99021, about 10 ng/ml FGF10, about 10 ng/ml KGF, about 10 ng/ml BMP4, and about 50 nM retinoic acid). In certain embodiments, the second culture medium may comprise insulin, an agonist of the EGF signaling pathway (e.g., EGF), a corticosteroid (e.g., hydrocortisone), a 3′,5′-cyclic adenosine monophosphate (cAMP) pathway activator (e.g., cholera toxin), and an inhibitor of ROCK (e.g., Y27632), (e.g., about 5 μg/ml insulin, about 0.1 ng/ml EGF, about 25 ng/ml hydrocortisone, about 8 ng/ml cholera toxin, and about 5 μM Y27632). After a period of time (e.g., about 2 to about 3 weeks), a lung progenitor cell culture is established that can be maintained by regular passaging for, e.g., six months or longer.

In certain embodiments, the present method does not require sorting for lineage-specific reporters or surface markers to enrich for desired lung lineages to isolate these prior to further differentiation.

The lung progenitor cells may be serially passaged every (about) 1 week, every (about) 2 weeks, every (about) 3 weeks, every (about) 4 weeks, every (about) 5 weeks, every (about) 3 days, every (about) 4 days, every (about) 5 days, every (about) 6 days, every (about) 8 days, every (about) 9 days, every (about) 10 days, every (about) 11 days, every (about) 12 days, every (about) 13 days, every (about) 15 days, every (about) 16 days, every (about) 17 days, every (about) 18 days, every (about) 19 days, or every (about) 20 days. The lung progenitor cells may be passaged for at least or about 1 passage, at least or about 2 passages, at least or about 3 passages, at least or about 4 passages, at least or about 5 passages, at least or about 6 passages, at least or about 7 passages, at least or about 8 passages, at least or about 9 passages, at least or about 10 passages, or more passages.

The lung progenitor cells may be cultured for 1 or more passages, for example, 1, 2, 3, 4, 5, 6, 7, 8, 9, 10, 15, 20, 25, 30 or more passages, for example, 20-30 passages, 30-35 passages, 32-40 passages or more. In some embodiments, an expanding cell population is split/passaged once a month, once every two weeks, once a week, twice a week, three times a week, four times a week, five times a week, six times a week or daily.

The present disclosure also provides lung progenitor cells which have been cultured for at least or about 1 week, at least or about 2 weeks, at least or about 3 weeks, at least or about 4 weeks, at least or about 5 weeks, at least or about 6 weeks, at least or about 7 weeks, at least or about 8 weeks, at least or about 9 weeks, at least or about 10 weeks, at least or about 12 weeks, at least or about 14 weeks, at least or about 16 weeks, at least or about 18 weeks, at least or about 20 weeks.

The cell culture medium used in the present method may be a serum-free medium or a serum-containing medium.

The lung progenitor cells may be cryopreserved, or thawed after cryopreservation.

The present disclosure provides for lung progenitor cells derived from mammalian pluripotent stem cells (e.g., human pluripotent stem cells or hPSCs), and methods for generating these cells. PSCs may comprise embryonic stem cells (ESCs) and/or induced pluripotent stem cells (iPSCs). Derived from the inner cell mass of the blastocyst, ESCs can be maintained in a pluripotent state in vitro and have the potential to generate every cell type in the organism. iPSCs may be generated by reprogramming somatic cells to a pluripotent state similar to ESCs, and can be patient-specific.

Producing anterior foregut endoderm cells from mammalian PSCs (e.g., step (a)), may last for about 2 days to about 8 days, about 3 days to about 7 days, about 3 days to about 6 days, about 3 days, or about 6 days. Producing anterior foregut endoderm cells from mammalian PSCs (e.g., step (a)) may be conducted, for example, at a time point ranging from day 3 to day 8, or from day 4 to day 6, counting from the start of culturing the PSCs (day 0).

Culturing the anterior foregut endoderm cells in a suspension culture to generate at least one lung bud organoid (LBO) (e.g., step (b)), may last for about 2 days to about 30 days, about 5 days to about 28 days, about 10 days to about 25 days, about 15 days to about 25 days, about 16 days to about 23 days, about 10 days to about 16 days, about 10 days to about 30 days, about 10 days to about 20 days, about 16 days, or about 23 days. Culturing the anterior foregut endoderm cells in a suspension culture to generate at least one lung bud organoid (LBO) (e.g., step (b)) may be conducted, for example, at a time point ranging from day 8 to day 30, or from day 10 to day 25, counting from the start of culturing the PSCs.

Dissociating and plating the LBO on a first cell culture substrate (e.g., in step (c)), may be conducted at a time point ranging from day 15 to day 30, or day 25, counting from the start of culturing the PSCs.

In certain embodiments, culturing the dissociated LBO on a first cell culture substrate (e.g., in step (c)), may last for about 20 days to about 200 days, about 30 days to about 180 days, about 50 days to about 160 days, about 100 days to about 200 days, about 20 days to about 50 days, about 20 days to about 30 days, about 10 days to about 30 days, or about 10 days to about 20 days. Culturing the dissociated LBO on a first cell culture substrate (e.g., in step (c)), may last shorter or longer than the above periods. Culturing the dissociated LBO to generate plated LBO (e.g., in step (c)) may be conducted, for example, at a time point ranging from day 20 to day 180, counting from the start of culturing the PSCs.

In certain embodiments, the plated LBO may be cultured on a second cell culture substrate in a first culture medium or a second culture medium (e.g., in step (d)) at a time point ranging from about day 20 to about day 180, or from about day 25 to about day 150, counting from the start of culturing the PSCs. The plated LBO may be cultured on a second cell culture substrate in a first culture medium or a second culture medium (e.g., in step (d)) for desired time periods, or indefinitely.

Stem cells (e.g., pluripotent stem cells, such as embryonic stem (ES) cells or induced pluripotent cells (iPSCs)) are subjected to a series of different culture steps to orchestrate differentiation of the stem cells into definitive endoderm (DE), anterior foregut endoderm (AFE) cells, and then into LBOs. In certain embodiments, LBOs (up to about 20-25 days in suspension culture) express sonic hedgehog (SHH) on the tips of budding epithelial structures.

The phenotypes of the various cells may be:
1. Embryonic stem cells or iPSC cells: undifferentiated.
2. Definitive endoderm: FOXA2+, cKIT+, CXCR4+, EPCAM+ (epithelial marker).
3. Anterior foregut endoderm: FOXA2+, SOX2+, EPCAM+, CDX2-.
4. Ventral anterior foregut endoderm: FOXA2+, SOX2^{low}, EPCAM+.
5. Lung bud organoids: FOXA2+NKX2.1+EPCAM+.

In certain embodiments, first, stem cells (e.g., hPSCs) are subjected to the embryoid bodies/primitive streak formation medium under conditions to induce differentiation of the pluripotent cells to definitive endoderm (DE). This first stage typically takes 4 days (day 0 to day 4 counting from the start of culturing the stem cells) and forms embryoid bodies having endoderm (e.g., as determined through expression of CXCR4 and c-kit). Second, day 5 to day 6, embryoid bodies are subjected to the anteriorization medium under conditions for the embryoid bodies to form anterior foregut patterning. Third, day 6 to day 20-25, cells are then subjected to the ventralization medium/branching medium under conditions that induce ventralization and ultimate production of lung bud organoids (LBOs). LBO formation may be determined by sonic hedgehog (SHH) expression on the tips of budding epithelial structures.

Such methods are not limited to a particular manner of accomplishing the directed differentiation of stem cells (e.g., PSCs) into anterior foregut endoderm cells. Indeed, any suitable method for producing anterior foregut endoderm cells from stem cells such as pluripotent stem cells (e.g., iPSCs or ESCs) is applicable to the methods described herein.

The present disclosure also provides for lung progenitor cells generated by the present methods, or a cell population comprising the lung progenitor cells generated by the present methods.

The present disclosure provides for lung progenitor cells, or a cell population, generated in vitro.

The lung progenitor cells, or cell population may comprise secretory-like cells, and airway basal-like cells. The lung progenitor cells, or cell population may further comprise fibroblasts, T cells and dendritic cells.

In certain embodiments, at least or about 10%, at least or about 20%, at least or about 30%, at least or about 40%, at least or about 50%, at least or about 60%, at least or about 70%, at least or about 80%, at least or about 90%, of the lung progenitor cells, or cell population, are secretory-like cells. In certain embodiments, at least or about 10%, at least or about 20%, at least or about 30%, at least or about 40%, at least or about 50%, at least or about 60%, at least or about 70%, at least or about 80%, at least or about 90%, of the lung progenitor cells, or cell population, are airway basal-like cells. In certain embodiments, about 70% to about 90% of the lung progenitor cells are airway basal-like cells, and about 10% to about 30% of the lung progenitor cells are secretory-like cells. In certain embodiments, about 80% of the lung progenitor cells are airway basal-like cells, and about 20% of the lung progenitor cells are secretory-like cells.

The lung progenitor cells, or cell population may comprise cells expressing MUC1, NOTCH3, UPK3A, KRT4, and KRT13, and cells expressing p63, KRT5, KRT17, ITGB4, JAG2 and DLK2. In certain embodiments, about 10% to about 30% of the lung progenitor cells are cells expressing MUC1, NOTCH3, UPK3A, KRT4, and KRT13, and about 70% to about 90% of the lung progenitor cells are cells expressing p63, KRT5, KRT17, ITGB4, JAG2 and DLK2. In certain embodiments, about 20% of the lung progenitor cells are cells expressing MUC1, NOTCH3, UPK3A, KRT4, and KRT13, and about 80% of the lung progenitor cells are cells expressing p63, KRT5, KRT17, ITGB4, JAG2 and DLK2.

In certain embodiments, at least or about 10%, at least or about 20%, at least or about 30%, at least or about 40%, at least or about 50%, at least or about 60%, at least or about 70%, at least or about 80%, at least or about 90%, of the lung progenitor cells, or cell population, are cells expressing MUC1, NOTCH3, UPK3A, KRT4, and KRT13.

In certain embodiments, at least or about 10%, at least or about 20%, at least or about 30%, at least or about 40%, at least or about 50%, at least or about 60%, at least or about 70%, at least or about 80%, at least or about 90%, of the lung progenitor cells, or cell population, are cells expressing p63, KRT5, KRT17, ITGB4, JAG2 and DLK2.

The lung progenitor cells, or cell population may comprise MUC1^{hi}CD104^{lo} cells and MUC1^{lo}CD104^{hi} cells.

In certain embodiments, at least or about 10%, at least or about 20%, at least or about 30%, at least or about 40%, at least or about 50%, at least or about 60%, at least or about 70%, at least or about 80%, at least or about 90%, of the lung progenitor cells, or cell population, are MLUC1^{hi}CD104^{lo} cells.

In certain embodiments, at least or about 10%, at least or about 20%, at least or about 30%, at least or about 40%, at least or about 50%, at least or about 60%, at least or about 70%, at least or about 80%, at least or about 90%, of the lung progenitor cells, or cell population, are MUC1^{lo}CD104^{hi} cells.

The present disclosure provides for a pharmaceutical composition comprising the present lung progenitor cells, or cell population.

The pharmaceutical composition may further comprise a pharmaceutically and/or physiologically acceptable vehicle or carrier, such as buffered saline or other buffers, e.g., HEPES, to maintain pH at appropriate physiological levels, and, optionally, other medicinal agents, pharmaceutical agents, stabilizing agents, buffers, carriers, adjuvants, diluents, etc. Exemplary physiologically acceptable carriers include sterile, pyrogen-free water and sterile, pyrogen-free, phosphate buffered saline.

The cell population, cell aggregates, or cell clusters may be dissociated by an enzymatic treatment. For example, the enzyme(s) may comprise at least one protease. The organoid, cell aggregates, or cell clusters may be dissociated by dispase, accutase, trypsin, and/or collagenase (e.g., collagenase I, II, III, and IV, etc.).

The present lung progenitor cells, cell population, or pharmaceutical composition may be engrafted, transplanted, or implanted into a subject. The present lung progenitor cells, cell population, or pharmaceutical composition may be administered to the subject by routes including, but not limited to, intranasal, direct delivery to a desired tissue/organ (e.g., the lung, airway or nasal cavity of a subject), oral, inhalation, intrabrochial, intratracheal, intravenous, intramuscular, subcutaneous, intradermal, and other parental routes of administration. The present lung progenitor cells, cell population, or pharmaceutical composition may be administered as an aerosol. The present lung progenitor cells, cell population, or pharmaceutical composition may be administered via a nebulizer. Additionally, routes of administration may be combined, if desired.

The lung progenitor cells may be non-syngeneic with the subject. The lung progenitor cells may be syngeneic with the subject. The lung progenitor cells may be allogeneic or xenogeneic with the subject. The lung progenitor cells may be autologous or allogeneic to the subject.

Also encompassed by the present disclosure is a method of treating a pulmonary disorder or injury in a subject in need thereof. The method may comprise administering to the subject an effective amount (e.g., a therapeutically effective amount) of the present lung progenitor cells, or pharmaceutical composition.

The method may comprise engrafting an effective amount (e.g., a therapeutically effective amount) of the present lung progenitor cells, or pharmaceutical composition into the lung, airway or nasal cavity of the subject. The engrafted cells may integrate into the epithelium.

The present disclosure provides for methods of using the present lung progenitor cells, or cell population, in a drug discovery screen; toxicity assay; research of tissue embryology, cell lineages, and differentiation pathways; gene expression studies including recombinant gene expression; research of mechanisms involved in tissue injury and repair; research of inflammatory and infectious diseases; studies of pathogenetic mechanisms; or studies of mechanisms of cell transformation and etiology of cancer.

The cell culture medium may comprise one or more factors selected from the group consisting of Wnt ligands, Wnt signaling activators (or Wnt agonists), BMPs, epidermal growth factors (EGFs), fibroblast growth factors (FGFs), and retinoic acid.

The cell culture medium may comprise one or more agonists of the Wnt signaling, FGF signaling, BMP signaling, and EGF signaling pathways. For example, the cell culture medium may comprise 1 or more, 2 or more, 3 or more, 4 or more, 5 or more, 6 or more, 7 or more, 8 or more, 9 or more, or 10 or more, agonists of the Wnt signaling, FGF signaling, BMP signaling, and EGF signaling pathways.

A Wnt agonist, or an agonist (or activator) of the Wnt signaling, may be used in one or more of the following steps: culturing the anterior foregut endoderm cells in a suspension culture to generate at least one lung bud organoid (LBO) (e.g., step (b)); culturing the dissociated LBO on a first cell culture substrate (e.g., step (c)); and in the first culture medium.

The “Wnt signaling activator” or “Wnt signaling agonist” as used herein refers to an agent that activates the Wnt signaling pathway.

The Wnt signaling pathway can include a series of events that occur when a Wnt protein binds to a cell-surface receptor of a Frizzled receptor family member. This results in the activation of Dishevelled family proteins which inhibit a complex of proteins that includes axin, GSK-3, and the protein APC to degrade intracellular β-catenin. The resulting enriched nuclear β-catenin enhances transcription by TCF/LEF family transcription factors.

A Wnt agonist (e.g., a small molecule or other agents) may be an agent that activates TCF/LEF-mediated transcription in a cell. A Wnt agonist can be an agent that binds and activates a Frizzled receptor family member including any and all of the Wnt family proteins, an inhibitor of intracellular β-catenin degradation, and an activator of TCF/LEF. The Wnt agonist may stimulate a Wnt activity in a cell by at least or about 10%, at least or about 20%, at least or about 30%, at least or about 50%, at least or about 70%, at least or about 90%, at least or about 100%, relative to a level of the Wnt activity in the absence of the agent, as assessed in the same cell type. As known to a skilled person, a Wnt activity can be determined by measuring the transcriptional activity of Wnt, for example by pTOPFLASH and pFOPFLASH Tcfluciferase reporter constructs (see, e.g., Korinek et al., 1997, Science 275:1784-1787).

A Wnt agonist may comprise a secreted glycoprotein including Wnt-1/Int-1; Wnt-2/Irp (Int-1-related Protein); Wnt-2b/13; Wnt-3/Int-4; Wnt-3a; Wnt-4; Wnt-5a; Wnt-5b; Wnt-6 (Kirikoshi H et al. 2001, Biochem. Biophys. Res. Com. 283: 798-805); Wnt-7a; Wnt-7b; Wnt-8a/8d; Wnt-8b; Wnt-9a/14; Wnt-9b/14b/15; Wnt-10a; Wnt-10b/12; Wnt-11; and Wnt-16.

Further Wnt agonists include the R-spondin family of secreted proteins, which is implicated in the activation and regulation of Wnt signaling pathway and which is comprised of 4 members (R-spondin 1 (NU206, Nuvelo, San Carlos, Calif.), R-spondin 2, R-spondin 3, and R-spondin-4); and Norrin (also called Norrie Disease Protein or NDP), which is a secreted regulatory protein that functions like a Wnt protein in that it binds with high affinity to the Frizzled-4 receptor and induces activation of the Wnt signaling pathway (Kestutis Planutis et al. (2007) BMC Cell Biol. 8: 12). Compounds that mimic the activity of R-spondin may be used as Wnt agonists. Lgr5 agonists such as agonistic anti-Lgr5 antibodies are examples of Wnt agonists that may be used.

A small molecule agonist of the Wnt signaling pathway, an aminopyrimidine derivative, may also be used as a Wnt agonist (Liu et al. (2005) Angew Chem Int Ed Engl. 44, 1987-90).

Examples of the Wnt signaling activator include glycogen synthase kinase (GSK) inhibitors such as GSK3 inhibitors. In some embodiments, activation of Wnt/beta-catenin signaling is achieved by inhibiting GSK3 phosphotransferase activity or GSK3 binding interactions. GSK3 inhibition can be achieved in a variety of ways including, but not limited to, providing small molecules that inhibit GSK3 phosphotransferase activity, RNA interference (RNAi such as small interfering RNAs or siRNAs, and short hairpin RNAs or shRNAs) against GSK3, and overexpression of dominant negative form of GSK3. Dominant negative forms of GSK3 are known in the art as described, e.g., in Hagen et al. (2002), J. Biol. Chem., 277(26):23330-23335, which describes a Gsk3 comprising an R96A mutation.

In some embodiments, GSK3 is inhibited by a small molecule that inhibits GSK3 phosphotransferase activity or GSK3 binding interactions. Suitable small molecule GSK3 inhibitors include, but are not limited to, CHIR99021, CHIR98014, BIO-acetoxime, 6-Bromoindirubin-3′-oxime (BIO), LiCl, SB216763, SB415286, AR A014418, Kenpaullone, 1-Azakenpaullone, Bis-7-indolylmaleimide, TWS119, and any combinations thereof.

GSK3 inhibitors also include lithium, and FRAT-family members and FRAT-derived peptides that prevent interaction of GSK3 with axin. An overview is provided by Meijer et al., (2004) Trends in Pharmacological Sciences 25, 471-480. Methods and assays for determining a level of GSK3 inhibition are known to a skilled person and comprise, for example, the methods and assay as described in Liao et al 2004, Endocrinology, 145(6): 2941-9.

In certain embodiments, the GSK3 activity may be inhibited by RNA interference targeting GSK3. For example, GSK3 expression levels can be knocked-down using siRNAs against GSK3, or a retroviral vector with an inducible expression cassette for GSK3, e.g., a Tet-inducible retroviral RNA interference (RNAi) system, or a cumate-inducible system.

In some embodiments, an agonist of Wnt signaling is Wnt3a, which mediates canonical Wnt signaling; any inducer of canonical Wnt signaling can be used, including, for example, Wnt/beta-catenin pathway agonists glycogen synthase kinase 3 beta (GSK3b) inhibitors, and casein kinase 1 (CK1) inhibitors.

Non-limiting examples of Wnt agonists include DNA encoding β-catenin (e.g., vectors encoding β-catenin, etc.), β-catenin polypeptides, one or more Wnt/β-catenin pathway agonists (e.g., Wnt ligands, DSH/DVL-1, -2, -3, LRP6N, WNT3A, WNT5A, and WNT3A), one or more glycogen synthase kinase 3β (GSK3β) inhibitors (e.g., lithium chloride (LiCl), Purvalanol A, olomoucine, alsterpaullone, kenpaullone, benzyl-2-methyl-1,2,4-thiadiazolidine-3,5-dione (TDZD-8), 2-thio(3-iodobenzyl)-5-(1-pyridyl)-[1,3,4]-oxadiazole, 2,4-dibenzyl-5-oxothiadiazolidine-3-thione (OTDZT), (2′Z,3′E)-6-Bromoindirubin-3′-oxime (BIO), α-4-Dibromoacetophenone (Tau Protein Kinase I (TPK I) Inhibitor), 2-Chloro-1-(4,5-dibromo-thiophen-2-yl)-ethanone, N-(4-Methoxybenzyl)-N′-(5-nitro-1,3-thiazol-2-yl)urea (AR-A014418), indirubin-5-sulfonamide; indirubin-5-sulfonic acid (2-hydroxyethyl)-amide indirubin-3′-monoxime; 5-iodo-indirubin-3′-monoxime; 5-fluoroindirubin; 5,5′-dibromoindirubin; 5-nitroindirubin; 5-chloroindirubin; 5-methylindirubin, 5-bromoindirubin, 4-Benzyl-2-methyl-1,2,4-thiadiazolidine-3,5-dione (TDZD-8), H-KEAPPAPPQSpP-NH2 (L803) and Myr-N-GKEAPPAPPOSpP-NH2 (L803-mts)), one or more anti-sense RNA or siRNA that bind specifically to GSK3, one or more casein kinase 1 (CK1) inhibitors (e.g., antisense RNA or siRNA that binds specifically to CK1 mRNA), protease inhibitors, and proteasome inhibitors.

In certain embodiments, the GSK3 inhibitor (e.g., CHIR99021 or any agent described herein) is used at a concentration ranging from about 1 μM to about 100 μM, from about 1 μM to about 50 μM, from about 1 μM to about 30 μM, from about 1 μM to about 20 μM, from about 1 μM to about 10 μM, at least or about 1 μM, at least or about 2 μM, at least or about 3 μM, at least or about 4 μM, at least or about 5 μM, at least or about 6 μM, at least or about 7 μM, at least or about 8 μM, at least or about 9 μM, at least or about 10 μM, at least or about 11 μM, at least or about 12 μM, at least or about 13 μM, at least or about 14 μM, at least or about 15 μM, at least or about 16 μM, at least or about 17 μM, at least or about 18 μM, at least or about 19 μM, or at least or about 20 μM, or higher concentrations. In another embodiment, the GSK3 inhibitor is used at a concentration ranging from about 0.1 μM to about 1 μM, e.g., at least or about 0.1 μM, at least or about 0.2 μM, at least or about 0.3 μM, at least or about 0.4 μM, at least or about 0.5 μM, at least or about 0.6 μM, at least or about 0.7 μM, at least or about 0.8 μM, at least or about 0.9 μM, or at least or about 1 μM.

An FGF agonist, or an agonist (or activator) of the FGF signaling, may be used in one or more of the following steps: culturing the anterior foregut endoderm cells in a suspension culture to generate at least one lung bud organoid (LBO) (e.g., step (b)); culturing the dissociated LBO on a first cell culture substrate (e.g., step (c)); and in the first culture medium.

The agonists of the FGF signaling include, but are not limited to, FGF7 or keratinocyte growth factor (KGF), FGF9, or FGF10. In some embodiments, other agonists of FGF signaling can be used, e.g., FGF1, FGF2, FGF3, FGF5, FGF6, FGF9, FGF11, FGF12, FGF13, FGF14, FGF15, FGF16, FGF17, FGF18, FGF19, FGF20, FGF21, FGF22, or FGF23.

For example, FGF (e.g., KGF or FGF10 or any FGF as described herein) may be at a concentration of about 1 ng/ml to 10 g/ml, 10 ng/ml to 1 g/ml, 10 ng/ml to 500 ng/ml, 10 ng/ml to 250 ng/ml, 10 ng/ml to 100 ng/ml, at least or about 1 ng/ml, at least or about 2 ng/ml, at least or about 3 ng/ml, at least or about 4 ng/ml, at least or about 5 ng/ml, at least or about 6 ng/ml, at least or about 7 ng/ml, at least or about 8 ng/ml, at least or about 9 ng/ml, at least or about 10 ng/ml, at least or about 11 ng/ml, at least or about 12 ng/ml, at least or about 13 ng/ml, at least or about 14 ng/ml, at least or about 15 ng/ml, at least or about 16 ng/ml, at least or about 17 ng/ml, at least or about 18 ng/ml, at least or about 19 ng/ml, at least or about 20 ng/ml, at least or about 25 ng/ml, at least or about 30 ng/ml, at least or about 35 ng/ml, at least or about 40 ng/ml, at least or about 45 ng/ml, at least or about 50 ng/ml, at least or about 55 ng/ml, at least or about 60 ng/ml, at least or about 65 ng/ml, at least or about 70 ng/ml, at least or about 75 ng/ml, at least or about 80 ng/ml, at least or about 85 ng/ml, at least or about 90 ng/ml, at least or about 95 ng/ml, or at least or about 100 ng/ml. In certain embodiments, FGF7 and/or FGF10 is/are at a concentration of about 25 ng/ml to 150 ng/ml, 50 ng/ml to 150 ng/ml, or 75 ng/ml to 150 ng/ml. In certain embodiments, KGF and/or FGF10 are present at a concentration of about 10 ng/ml.

A BMP agonist, or an agonist (or activator) of the BMP signaling, may be used in one or more of the following steps: culturing the anterior foregut endoderm cells in a suspension culture to generate at least one lung bud organoid (LBO) (e.g., step (b)); culturing the dissociated LBO on a first cell culture substrate (e.g., step (c)); and in the first culture medium.

The agonists of BMP signaling include, but are not limited to, BMP1, BMP2, BMP3, BMP4, BMP5, BMP6, BMP7, BMP8, BMP9, BMP10, BMP11, BMP12, BMP13, BMP14, BMP15, BMP16, BMP17, BMP18, BMP19, or BMP20. In certain embodiments, any of BMP2-7 is/are used, e.g., BMP4.

BMP may be present at a concentration of about 1 ng/ml to 10 g/ml, 10 ng/ml to 1 g/ml, 10 ng/ml to 500 ng/ml, 10 ng/ml to 250 ng/ml, or 10 ng/ml to 100 ng/ml. In certain embodiments, BMP4 is present at a concentration of about 25 ng/ml to 150 ng/ml, 50 ng/ml to 150 ng/ml or 75 ng/ml to 150 ng/ml. In certain embodiments, one or more BMP is/are present in cultures at a concentration of about 0.5 ng/ml, about 3 ng/ml, and/or about 10 ng/ml. For example, BMP (e.g., BMP4 or any BMP as described herein) may be present at a concentration of about 1 ng/ml to 10 g/ml, 10 ng/ml to 1 g/ml, 10 ng/ml to 500 ng/ml, 10 ng/ml to 250 ng/ml, 10 ng/ml to 100 ng/ml, at least or about 1 ng/ml, at least or about 2 ng/ml, at least or about 3 ng/ml, at least or about 4 ng/ml, at least or about 5 ng/ml, at least or about 6 ng/ml, at least or about 7 ng/ml, at least or about 8 ng/ml, at least or about 9 ng/ml, at least or about 10 ng/ml, at least or about 11 ng/ml, at least or about 12 ng/ml, at least or about 13 ng/ml, at least or about 14 ng/ml, at least or about 15 ng/ml, at least or about 16 ng/ml, at least or about 17 ng/ml, at least or about 18 ng/ml, at least or about 19 ng/ml, at least or about 20 ng/ml, at least or about 25 ng/ml, at least or about 30 ng/ml, at least or about 35 ng/ml, at least or about 40 ng/ml, at least or about 45 ng/ml, at least or about 50 ng/ml, at least or about 55 ng/ml, at least or about 60 ng/ml, at least or about 65 ng/ml, at least or about 70 ng/ml, at least or about 75 ng/ml, at least or about 80 ng/ml, at least or about 85 ng/ml, at least or about 90 ng/ml, at least or about 95 ng/ml, or at least or about 100 ng/ml. In certain embodiments, BMP (e.g., BMP4 or any BMP as described herein) is present at a concentration of about 25 ng/ml to 150 ng/ml, 50 ng/ml to 150 ng/ml, or 75 ng/ml to 150 ng/ml. In certain embodiments, BMP (e.g., BMP4 or any BMP as described herein) is present at a concentration of about 10 ng/ml.

Retinoid acid, or its derivatives, may be used in one or more of the following steps: culturing the anterior foregut endoderm cells in a suspension culture to generate at least one lung bud organoid (LBO) (e.g., step (b)); culturing the dissociated LBO on a first cell culture substrate (e.g., step (c)); and in the first culture medium. Retinoic acid may be all-trans retinoic acid, 9-cis retinoic acid, 13-cis retinoic acid, etc.

In certain embodiments, retinoic acid or its derivative is used at a concentration ranging from about 1 nM to about 100 nM, from about 20 nM to about 80 nM, from about 30 nM to about 60 nM, at least or about 10 nM, at least or about 20 μM, at least or about 30 nM, at least or about 40 nM, at least or about 50 nM, at least or about 60 nM, at least or about 70 nM, at least or about 80 nM, at least or about 90 nM, at least or about 100 nM, at least or about 15 nM, at least or about 25 nM, at least or about 35 nM, at least or about 45 nM, at least or about 55 nM, at least or about 65 nM, at least or about 75 nM, at least or about 85 nM, at least or about 95 nM, or at least or about 5 nM, or higher concentrations. In another embodiment, retinoic acid is used at a concentration ranging from about 40 nM to about 60 nM, e.g., at least or about 30 nM, at least or about 70 nM, at least or about 41 nM, at least or about 42 nM, at least or about 43 nM, at least or about 44 nM, at least or about 46 nM, at least or about 47 nM, at least or about 48 nM, or at least or about 49 nM.

An inhibitor of ROCK (or ROCK inhibitor) may be used in the first culture medium and/or the second culture medium.

The inhibitor of ROCK (or ROCK inhibitor) may be any agent that decreases the level and/or activity of ROCK. The ROCK inhibitors can be small organic or inorganic molecules; saccharides; oligosaccharides; polysaccharides; biological macromolecules, e.g., peptides, proteins, and peptide analogs and derivatives; peptidomimetics; nucleic acids and nucleic acid analogs and derivatives (including but not limited to microRNAs, siRNAs, shRNAs, antisense RNAs, a ribozymes, and aptamers); an extract made from biological materials such as bacteria, plants, fungi, or animal cells; animal tissues; naturally occurring or synthetic compositions; and any combinations thereof.

ROCK inhibitors include, but are not limited to, a small organic molecule ROCK inhibitor such as N-[(1S)-2-hydroxy-1-phenylethyl]-N′-[4-(4-pyridinyl)phenyl]-urea (AS1892802), fasudil hydrochloride (also known as HA 1077), N-[3-[[2-(4-amino-1,2,5-oxadiazol-3-yl)-1-ethyl-1H-imidazo[4,5-c]pyridin-6-yl]oxy]phenyl]-4-[2-(4-morpholinyl)ethoxy]benzamide (GSK269962), 4-[4-(Trifluoromethyl)phenyl]-N-(6-Fluoro-1H-indazol-5-yl)-2-methyl-6-oxo-1,4,5,6-tetrahydro-3-pyridinecarboxamide (GSK 429286), (5)-(+)-2-Methyl-1-[(4-methyl-5-isoquinolinyl)sulfonyl]-hexahydro-1H-1,4-diazepine dihydrochloride (H 1152 dihydrochloride), (S)-(+)-4-Glycyl-2-methyl-1-[(4-methyl-5-isoquinolinyl)sulfonyl]-hexahydro-1H-1,4-diazepine dihydrochloride (glycyl-H 1152 dihydrochloride), N-[(3-Hydroxyphenyl)methyl]-N′-[4-(4-pyridinyl)-2-thiazolyl]urea dihydrochloride (RKI 1447 dihydrochloride), (3S)-1-[[2-(4-Amino-1,2,5-oxadiazol-3-yl)-1-ethyl-1H-imidazo[4,5-c]pyridin-7-yl]carbonyl]-3-pyrrolidinamine dihydrochloride (SB772077B dihydrochloride), N-[2-[2-(Dimethylamino)ethoxy]-4-(1H-pyrazol-4-yl)phenyl-2,3-dihydro-1,4-benzodioxin-2-carboxamide dihydrochloride (SR 3677 dihydrochloride), and trans-4-[(1R)-1-Aminoethyl]-N-4-pyridinylcyclohexanecarboxamide dihydrochloride (Y-27632 dihydrochloride), N-Benzyl[2-(pyrimidin-4-yl)amino]thiazole-4-carboxamide (Thiazovivin), a isoquinolinesulfonamide compound (Rho Kinase Inhibitor), N-(4-Pyridyl)-N′-(2,4,6-trichlorophenyl)urea (Rho Kinase Inhibitor II), 3-(4-Pyridyl)-1H-indole (Rho Kinase Inhibitor III, Rockout), and 4-pyrazoleboronic acid pinacol ester; a Rock antibody such as Rock-1 (B1), Rock-1 (C-19), Rock-1 (H-11), Rock-1 (G-6), Rock-1 (H-85), Rock-1 (K-18), Rock-2 (C-20), Rock-2 (D-2), Rock-2 (D-11), Rock-2 (N-19), Rock-2 (H-85), Rock-2 (30-J) (commercially available from Santa Cruz Biotechnology); a ROCK CRISPR/Cas9 knockout plasmid such as Rock-1 CRISPR/Cas9 KO plasmid (h), Rock-2 CRISPR/Cas9 KO plasmid (h), Rock-1 CRISPR/Cas9 KO plasmid (m), Rock-2 CRISPR/Cas9 KO plasmid (m); a ROCK siRNA, shRNA plasmid and/or shRNA lentiviral particle gene silencer such as Rock-1 siRNA (h): sc-29473, Rock-1 siRNA (m): sc-36432, Rock-1 siRNA (r): sc-72179, Rock-2 siRNA (h): sc-29474, Rock-2 siRNA (m): sc-36433, Rock-2 siRNA (r): se-108088 (commercially available from Santa Cruz Biotechnology).

In certain embodiments, the ROCK inhibitor decreases the level and/or activity of ROCK in cells or cell culture medium by at least or about 5%, at least or about 10%, at least or about 20%, at least or about 30%, at least or about 40%, at least or about 50%, at least or about 60%, at least or about 70%, at least or about 80%, at least or about 90%, or at least or about 95%. In one embodiment, a ROCK inhibitor may completely inhibit the level and/or activity of ROCK in the cells or cell culture medium.

The ROCK inhibitor may be Y27632 ((1R,4r)-4-((R)-1-aminoethyl)-N-(pyridin-4-yl)cyclohexanecarboxamide). Y27632 may have the following structure.

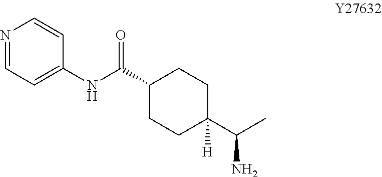 [SMILES: C[C@@H](N)[C@H]1CC[C@H](C(=O)Nc2ccncc2)CC1]

In certain embodiments, the ROCK inhibitor (e.g., Y27632 or any other agent described herein) is used at a concentration ranging from about 0.1 μM to about 100 μM, from about 1 μM to about 50 μM, from about 0.5 μM to about 25 μM, from about 1 μM to about 20 μM, from about 1 μM to about 10 μM, from about 5 μM to about 15 μM, from about 5 μM to about 10 μM, from about 1 μM to about 30 μM, e.g., at least or about 1 μM, at least or about 2 μM, at least or about 3 μM, at least or about 4 μM, at least or about 5 μM, at least or about 6 μM, at least or about 7 μM, at least or about 8 μM, at least or about 9 μM, at least or about 10 μM, at least or about 11 μM, at least or about 12 μM, at least or about 13 μM, at least or about 14 μM, at least or about 15 μM, at least or about 16 μM, at least or about 17 μM, at least or about 18 μM, at least or about 19 μM, at least or about 20 μM, at least or about 21 μM, at least or about 22 μM, at least or about 23 μM, at least or about 24 μM, at least or about 25 μM, at least or about 26 μM, at least or about 27 μM, at least or about 28 μM, at least or about 29 μM, or at least or about 30 μM, or higher concentrations. In another embodiment, the ROCK inhibitor is used at a concentration ranging from about 0.1 μM to about 1 μM, e.g., at least or about 0.1 μM, at least or about 0.2 μM, at least or about 0.3 μM, at least or about 0.4 μM, at least or about 0.5 μM, at least or about 0.6 μM, at least or about 0.7 μM, at least or about 0.8 μM, at least or about 0.9 μM, or at least or about 1 μM.

An effective amount of the ROCK inhibitor (e.g., Y27632 or any agent described herein) for use in the present methods can be, for example, between about 0.1 μM and about 110 μM. In some aspects, an effective amount of ROCK inhibitor (e.g., Y27632 or any agent described herein) is 10 μM or 5 μM.

An EGF agonist, or an agonist (or activator) of the EGF signaling, may be used in the second culture medium. For example, the EGF agonist (e.g., EGF) may be at a concentration of about 0.05 ng/ml to 1 ng/ml, about 0.05 ng/ml to 0.8 ng/ml, about 0.05 ng/ml to 0.6 ng/ml, about 0.05 ng/ml to 0.5 ng/ml, about 0.1 ng/ml to 20 g/ml, about 1 ng/ml to 10 g/ml, 10 ng/ml to 1 g/ml, 10 ng/ml to 500 ng/ml, 10 ng/ml to 250 ng/ml, or 10 ng/ml to 100 ng/ml. In preferred embodiments, EGF is present in cultures at a concentration of about 25 ng/ml to 150 ng/ml, 50 ng/ml to 150 ng/ml or 75 ng/ml to 150 ng/ml. In one embodiment, the EGF agonist (e.g., EGF) is present in the second culture medium at a concentration of about 0.1 ng/ml.

A steroid (e.g., a corticosteroid) may be used in the second culture medium. The steroid may be a glucocorticoid or a mineralocorticoid. Exemplary steroids include, but are not limited to, hydrocortisone (cortisol), dexamethasone, dexamethasone derivatives, beclometasone, betamethasone, fluocortolone, halometasone, mometasone, prednisone, prednisone derivatives, fludrocortisone, hydrocortisone acetate, cortisone acetate, tixocortol pivalate, prednisolone, methylprednisolone, corticosterone, cortisone, aldosterone, amcinonide, budesonide, desonide, fluocinolone acetonide, fluocinonide, halcinonide, triamcinolone acetonide, alclometasone dipropionate, betamethasone dipropionate, betamethasone valerate, clobetasol propionate, clobetasone butyrate, fluprednidene acetate, mometasone furoate, ciclesonide, cortisone acetate, hydrocortisone aceponate, hydrocortisone acetate, hydrocortisone buteprate, hydrocortisone butyrate, hydrocortisone valerate, prednicarbate, and tixocortol pivalate.

The concentration of the corticosteroid (e.g., hydrocortisone or any steroid as described herein) may range from about 10 nM to about 10 μM, from about 10 nM to about 5 μM, from about 10 nM to about 1 μM, from about 10 nM to about 500 nM, from about 20 nM to about 400 nM, from about 30 nM to about 300 nM, from about 40 nM to about 250 nM, from about 50 nM to about 200 nM, from about 50 nM to about 180 nM, from about 50 nM to about 160 nM, from about 50 nM to about 150 nM, from about 50 nM to about 130 nM, from about 60 nM to about 300 nM, from about 60 nM to about 250 nM, from about 60 nM to about 200 nM, from about 60 nM to about 180 nM, from about 60 nM to about 160 nM, from about 60 nM to about 150 nM, from about 60 nM to about 130 nM, from about 80 nM to about 300 nM, from about 80 nM to about 250 nM, from about 80 nM to about 200 nM, from about 80 nM to about 180 nM, from about 80 nM to about 160 nM, from about 80 nM to about 150 nM, from about 80 nM to about 130 nM, from about 100 nM to about 300 nM, from about 100 nM to about 250 nM, from about 100 nM to about 200 nM, from about 100 nM to about 180 nM, from about 100 nM to about 160 nM, from about 100 nM to about 150 nM, from about 100 nM to about 130 nM, at least or about 10 nM, at least or about 20 nM, at least or about 30 nM, at least or about 40 nM, at least or about 50 nM, at least or about 60 nM, at least or about 70 nM, at least or about 80 nM, at least or about 90 nM, at least or about 100 nM, at least or about 110 nM, at least or about 120 nM, at least or about 125 nM, at least or about 127 nM, at least or about 130 nM, at least or about 140 nM, at least or about 150 nM, at least or about 15 nM, at least or about 25 nM, at least or about 35 nM, at least or about 45 nM, at least or about 55 nM, at least or about 65 nM, at least or about 75 nM, at least or about 85 nM, at least or about 95 nM, or at least or about 5 nM.

The concentration of the corticosteroid (e.g., hydrocortisone or any steroid as described herein) may range from about 10 ng/ml to about 100 ng/ml, from about 10 ng/ml to about 90 ng/ml, from about 10 ng/ml to about 80 ng/ml, from about 10 ng/ml to about 70 ng/ml, from about 10 ng/ml to about 60 ng/ml, from about 10 ng/ml to about 50 ng/ml, from about 20 ng/ml to about 100 ng/ml, from about 20 ng/ml to about 90 ng/ml, from about 20 ng/ml to about 80 ng/ml, from about 20 ng/ml to about 70 ng/ml, from about 20 ng/ml to about 60 ng/ml, from about 20 ng/ml to about 50 ng/ml, from about 20 ng/ml to about 30 ng/ml, from about 30 ng/ml to about 100 ng/ml, from about 30 ng/ml to about 90 ng/ml, from about 30 ng/ml to about 80 ng/ml, from about 30 ng/ml to about 70 ng/ml, from about 30 ng/ml to about 60 ng/ml, from about 30 ng/ml to about 50 ng/ml, from about 40 ng/ml to about 100 ng/ml, from about 40 ng/ml to about 90 ng/ml, from about 40 ng/ml to about 80 ng/ml, from about 40 ng/ml to about 70 ng/ml, from about 40 ng/ml to about 60 ng/ml, from about 40 ng/ml to about 50 ng/ml, at least or about 10 ng/ml, at least or about 15 ng/ml, at least or about 20 ng/ml, at least or about 25 ng/ml, at least or about 30 ng/ml, at least or about 40 ng/ml, at least or about 50 ng/ml, at least or about 60 ng/ml, at least or about 70 ng/ml, at least or about 80 ng/ml, at least or about 90 ng/ml, or at least or about 100 ng/ml.

Insulin may be used in the second culture medium.

The concentration of insulin may range from about 0.1 μg/ml to about 100 μg/ml, about 0.5 μg/ml to about 50 μg/ml, about 1 μg/ml to about 50 μg/ml, about 1 μg/ml to about 40 μg/ml, about 1 μg/ml to about 30 μg/ml, about 1 μg/ml to about 20 μg/ml, about 1 μg/ml to about 10 μg/ml, about 2 μg/ml to about 20 μg/ml, about 2 μg/ml to about 10 μg/ml, about 5 μg/ml to about 10 μg/ml, at least or about 1 μg/ml, at least or about 2 μg/ml, at least or about 3 μg/ml, at least or about 4 μg/ml, at least or about 5 μg/ml, at least or about 6 μg/ml, at least or about 7 μg/ml, at least or about 8 μg/ml, at least or about 9 μg/ml, or at least or about 10 μg/ml.

A cAMP pathway activator may be used in the second culture medium. The cAMP pathway activator may be any suitable activator which increases the levels of cAMP in a cell. The cAMP pathway involves activation of many types of hormones and neurotransmitter G-protein coupled receptors. Binding of the hormone or neurotransmitter to its membrane-bound receptor induces a conformational change in the receptor that leads to activation of the α-subunit of the G-protein. The activated G subunit stimulates, while the non-activated G subunit inhibits, adenylyl cyclase. Stimulation of adenylyl cyclase catalyzes the conversion of cytoplasmic ATP to cAMP, thus increasing the levels of cAMP in the cell.

The cAMP pathway activator may be, for example, an adenylyl cyclase activator. Examples of suitable adenylyl cyclase activators include forskolin, a forskolin analogue and cholera toxin. In some embodiments, the cAMP pathway activator is cholera toxin. In some embodiments, the cAMP pathway activator is forskolin. In some embodiments the cAMP pathway activator may be a cAMP analog, for example 8-bromo-cAMP. In some embodiments, the cAMP pathway activator is NKH477.

In some embodiments, the cAMP pathway activator (e.g., cholera toxin, or any other cAMP pathway activator as described herein) is used at a concentration ranging from about 10 nM to about 500 μM, from about 10 nM to about 100 μM, from about 1 μM to about 50 μM, from about 1 μM to about 25 μM, from about 5 μM to about 1000 μM, from about 5 μM to about 500 μM, from about 5 μM to about 100 μM, from about 5 μM to about 50 μM, from about 5 μM to about M, from about 10 μM to about 1000 μM, from about 10 μM to about 500 μM, from about 10 μM to about 100 μM, from about 10 μM to about 50 μM, from about 10 μM to about 25 μM, from about 10 μM to about 1 mM, from about 10 μM to about 900 μM, from about 10 μM to about 800 μM, from about 10 μM to about 700 μM, from about 10 μM to about 600 μM, from about 10 μM to about 500 μM, from about 10 μM to about 400 μM, from about 10 μM to about 300 μM, from about 10 μM to about 200 μM, from about 10 μM to about 100 μM, from about 50 μM to about 1 mM, from about 50 μM to about 900 μM, from about 50 μM to about 800 μM, from about 50 μM to about 700 μM, from about 50 μM to about 600 μM, from about 50 μM to about 500 μM, from about 50 μM to about 400 μM, from about 50 μM to about 300 μM, from about 50 μM to about 200 μM, or from about 50 μM to about 100 μM. In some embodiments the cAMP pathway activator is used at a concentration of at least or about 10 nM, at least or about 20 nM, at least or about 50 nM, at least or about 100 nM, at least or about 200 nM, at least or about 500 nM, at least or about 1 μM, at least or about 2 μM, at least or about 5 μM, at least or about 10 μM, at least or about 20 μM, at least or about 30 μM, at least or about 40 μM, at least or about 50 μM, at least or about 60 μM, at least or about 70 μM, at least or about 80 μM, at least or about 90 μM, at least or about 110 μM, at least or about 120 μM, at least or about 130 μM, at least or about 140 μM, at least or about 150 μM, at least or about 160 μM, or at least or about 100 μM (0.1 mM).

For example, NKH477 can in some embodiments be used at a concentration of between about 100 nM and about 10 μM, or at a concentration of about 100 nM, about 1 μM or about 10 μM. Forskolin can in some embodiments be used at a concentration of between about 1 μM and about 100 μM, or at a concentration of about 1 μM, about 10 μM or about 100 μM.

In some embodiments, the cAMP pathway activator (e.g., cholera toxin, or any other cAMP pathway activator as described herein) is used a concentration of between about 1 ng/ml and about 500 ng/ml, about 1 ng/ml and about 100 ng/ml, about 1 ng/ml and about 80 ng/ml, about 1 ng/ml and about 50 ng/ml, about 1 ng/ml and about 20 ng/ml, about 1 ng/ml and about 10 ng/ml, about 10 ng/ml and about 100 ng/ml, about 50 ng/ml and about 100 ng/ml, at least or about 1 ng/ml, at least or about 2 ng/ml, at least or about 5 ng/ml, at least or about 8 ng/ml, at least or about 10 ng/ml, at least or about 20 ng/ml, at least or about 30 ng/ml, at least or about 40 ng/ml, at least or about 50 ng/ml, at least or about 60 ng/ml, at least or about 70 ng/ml, at least or about 80 ng/ml, at least or about 90 ng/ml, at least or about 100 ng/ml, at least or about 200 ng/ml, at least or about 300 ng/ml, at least or about 400 ng/ml, or at least or about 500 ng/ml.

The first biomolecule and second biomolecule may be identical or may be different.

The first biomolecule or second biomolecule may comprise one or more proteins, polypeptides, and/or peptides. The first biomolecule or second biomolecule may include one or more extracellular matrix (ECM) proteins. The first biomolecule or second biomolecule may include, but are not limited to, a solubilized basement membrane preparation from the Engelbreth-Holm-Swarm (EHS) mouse sarcoma, Matrigel, fibronectin, collagen (e.g., collagen I, collagen IV, etc.), collagen derivatives, gelatin, laminin, heparan sulfate proteoglycans, entactin/nidogen, cellulose, cellulose derivatives, cellulose polymers, proteoglycans, heparin sulfate, chondroitin sulfate, keratin sulfates, hyaluronic acid, elastin, fibrin, chitosan, alginate, vinculin, agar, agarose, hyaluronic acid, and combinations thereof. The first biomolecule or second biomolecule may comprise one or more polymers including, but not limited to: polyethylene-imine and dextran sulfate, poly(vinylsiloxane)ecopolymerepoly-ethyleneimine, phosphorylcholine, poly(ethylene glycol), poly(lactic-glycolic acid), poly(lactic acid), polyhydroxyvalerte and copolymers, polyhydroxybutyrate and copolymers, polydiaxanone, polyanhydrides, polypeptides, poly(orthoesters), polyesters, and combinations thereof. The first biomolecule or second biomolecule may comprise one or more matrices described in Gjorevsky et al, Nature, 2016, 539(7630):560-564 and DiMarco et al., Biomater Sci. 2015, 3(10):1376-85.

In one embodiment, the first biomolecule or second biomolecule may comprise a gelatinous extracellular protein mixture secreted by Engelbreth-Holm-Swarm (EHS) mouse sarcoma cells. In one embodiment, the first biomolecule or second biomolecule may comprise Matrigel. Matrigel may comprise laminin, collagen IV, heparan sulfate proteoglycans, entactin/nidogen, TGF-beta, epidermal growth factor, insulin-like growth factor, fibroblast growth factor, tissue plasminogen activator, or combinations thereof.

The first biomolecule or second biomolecule may comprise (consist essentially of, or consist of) a naturally derived biopolymer matrix, a synthetic ECM analogue matrix, a hydrogel, a polyethylene glycol (PEG) hydrogel, an RGD functionalized PEG hydrogel, a polyacrylate hydrogel, a hydrogel having a cross-linked hydrophilic polymer functionalized with an RGD-containing peptide, etc.

Hydrogels include naturally derived hydrogels and synthetic hydrogels. Naturally derived hydrogels and synthetic hydrogels may be mixed to form hybrid hydrogels.

Naturally derived hydrogels may include, but are not limited to, Matrigel. Naturally derived hydrogels may be derived from decellularized tissue extracts. Extracellular matrix may be collected from a specific tissue and may be used as or combined with a hydrogel material to be used to support cells of that tissue type. See, e.g., Skardal et al., Tissue Specific Synthetic ECM Hydrogels for 3-D in vitro Maintenance of Hepatocyte Function, Biomaterials 33 (18): 4565-75 (2012). Chitosan hydrogel is an example of a naturally derived hydrogel that is degradable and supportive for several different cell types. See, e.g., Moura et al., In Situ Forming Chitosan Hydrogels Prepared via Ionic/Covalent Co-Cross-Linking, Biomacromolecules 12 (9): 3275-84 (2011). Hyaluronic acid hydrogels may also be used. See, e.g., Skardal et al., A hydrogel bioink toolkit for mimicking native tissue biochemical and mechanical properties in bioprinted tissue constructs, Acta Biomater. 25: 24-34 (2015).

Synthetic hydrogels may be produced from a variety of materials (e.g., polyethylene glycol). By combining natural components, such as extracellular matrix molecules (e.g., extracellular matrix proteins), with synthetic hydrogels, hybrid hydrogels can be produced. See, e.g., Salinas et al., Chondrogenic Differentiation Potential of Human Mesenchymal Stem Cells Photoencapsulated within Poly(Ethylene Glycol)-Arginine-Glycine-Aspartic Acid-Serine Thiol-Methacrylate Mixed-Mode Networks, Tissue Engineering 13 (5): 1025-34 (2007).

A hydrogel may be a matrix comprising a network of hydrophilic polymer chains. A biofunctional hydrogel may be a hydrogel that contains bio-adhesive (or bioactive) molecules, and/or cell signaling molecules that interact with living cells to promote cell viability and a desired cellular phenotype. Biofunctional hydrogels may also be referred to as bioactive. Examples of bio-adhesive molecules include, but are not limited to, fibronectin, vitronectin, bone sialoprotein, laminin, collagen and elastin. Examples of bio-adhesive molecules include cell adhesion peptides such as fibronectin-derived RGD. The hydrogels may comprise a hydrophilic polymer crosslinked with a functional molecule, where the functional molecule may comprise an oligopeptide, a small molecule, a protein, an oligo- or polysaccharides, or an oligo-nucleotides or poly-nucleotides. The functional molecule may be an RGD-containing ligand such as fibronectin or a functional variant thereof, where the functional variant of fibronectin may be a linear, branched or cyclic peptide.

In some embodiments, the hydrophilic polymer may be polyethylene glycol, polyoxazoline, polyaliphatic polyurethanes, polyether polyurethanes, polyester polyurethanes, polyethylene copolymers, polyamides, polyvinyl alcohols, polyethylene oxide, polypropylene oxide, polypropylene glycol, polytetramethylene oxide, polyvinyl pyrrolidone, polyacrylamide, polyhydroxy ethyl acrylate, poly(hydroxyethyl methacrylate), or mixtures or co-polymers thereof.

Hydrogel precursors may be linear PEG molecules, or multi-arm PEG hydrogel precursor molecules, such as those bearing 4-arms or 8-arms. Hydrogel precursors may be PEG hydrogel precursor molecules with molecular weight of 10-40 kDa. U.S. Pat. No. 10,934,529, the disclosure of which is incorporated herein by reference.

In certain embodiments, the first biomolecule or second biomolecule may be a biomatrix scaffold. The biomatrix scaffold may comprise collagens, fibronectins, laminins, nidogen/entactin, elastin, proteogylcans, glycosaminoglycans, growth factors, cytokines or combinations thereof. Biomatrix scaffold may be an isolated tissue extract enriched in extracellular matrix, which retains many or most of the collagens and collagen-bound factors found naturally in the biological tissue. Exemplary collagens include all types of collagens, such as Type I through Type XXIX collagens. U.S. Pat. No. 10,246,678, the disclosure of which is incorporated herein by reference.

A solution (e.g., an aqueous solution) of the first biomolecule or second biomolecule may be used to coat the first cell culture substrate or second cell culture substrate. The solution then solidifies to form a coating on the first cell culture substrate or second cell culture substrate. The concentration of the first biomolecule or second biomolecule in the coating may range from about 30% to about 100%, from about 40% to about 100%, from about 50% to about 100%, at least or about 20%, at least or about 30%, at least or about 40%, at least or about 50%, at least or about 60%, at least or about 70%, at least or about 80%, at least or about 90%, or about 100%.

The first cell culture substrate and second cell culture substrate may be identical or may be different.

The first cell culture substrate or second cell culture substrate may be a cell culture plate, a cell culture dish, a cell culture flask, a slide, a cell culture vessel, a cell culture container, a single-well plate, or a multi-well plate.

A stem cell may refer to a totipotent, pluripotent, multipotent, oligopotent or unipotent cell that can undergo self-renewing cell division to give rise to phenotypically and genotypically identical daughter cells for an indefinite time and can ultimately differentiate into at least one final cell type. The term “stem cell” may mean a cell derived from any source of tissue or organ and can replicate as undifferentiated or lineage committed cells and have the potential to differentiate into at least one, preferably multiple, cell lineages.

Examples of stem cells include totipotent, pluripotent, multipotent, oligopotent and unipotent stem cells (e.g., progenitor cells). Examples of pluripotent stem cells include embryonic stem cells, embryonic germ cells, embryonic carcinoma cells, and induced pluripotent stem cells (iPSCs). Non-limiting examples of stem cells include embryonic stem cells, fetal stem cells, and adult (or somatic) stem cells. Stem cells can be obtained commercially, or obtained/isolated directly from patients, or from any other suitable source.

A stem cell may also be undifferentiated or partially differentiated precursor cells, such as embryonic germ cells, mesenchymal stem cells, multipotent adult stem cells, etc.

In one embodiment, the stem cell is a human stem cell.

Pluripotent stem cells (PSCs) may include embryonic stem cells (ESCs) and induced pluripotent stem cells (iPSCs). In certain embodiments, embryonic stem cells or iPSC cells are undifferentiated pluripotent stem cells, expressing OCT4, SOX2, NANOG, and SSEA4.

ESCs may have unlimited self-renewal and multipotent and/or pluripotent differentiation potential, thus possessing the capability of developing into any organ, tissue type or cell type. These cells can be derived from the inner cell mass of the blastocyst, or from the primordial germ cells from a post-implantation embryo (embryonal germ cells or EG cells). Evans et al. (1981) Nature 292:154-156; Matsui et al. (1991) Nature 353:750-2; Thomson et al. (1995) Proc. Natl. Acad. Sci. USA. 92:7844-8; Thomson et al. (1998) Science 282:1145-1147; and Shamblott et al. (1998) Proc. Natl. Acad. Sci. USA 95:13726-31.

“Induced pluripotent stem cells,” commonly abbreviated as iPS cells or iPSCs, refer to a type of pluripotent stem cell artificially prepared from a non-pluripotent cell, for example an adult somatic cell, or terminally differentiated cell, such as a fibroblast, a hematopoietic cell, a myocyte, a neuron, an epidermal cell, or the like, by introducing certain factors, referred to as reprogramming factors. iPSCs may be generated by reprogramming somatic cells to a pluripotent state. In one aspect, the iPSC is derived from a fibroblast cell. For example, patient fibroblast cells can be collected from the skin biopsy and transformed into iPS cells. Dimos J T et al. (2008) Induced pluripotent stem cells generated from patients with ALS can be differentiated into motor neurons. Science 321: 1218-1221; Nature Reviews Neurology 4, 582-583. Luo et al., Generation of induced pluripotent stem cells from skin fibroblasts of a patient with olivopontocerebellar atrophy, Tohoku J. Exp. Med. 2012, 226(2): 151-9.

In certain embodiments, lung progenitor cells may be generated from a patient-specific source (e.g., iPSC cells), which can provide cell-based regenerative treatments for repopulating healthy lung tissue in diseased patient lungs.

In certain embodiments, the iPSC cells may be from a subject having at least one mutation in a lung disease-associated gene, and the iPSC cells have been genetically altered to correct the gene mutation. In one embodiment, the iPSCs may be genetically altered via the CRISPR/Cas system. In one embodiment, the CRISPR/Cas9 system is used to introduce patient mutations into the stem cell.

The present lung progenitor cells, cell population, or pharmaceutical composition may be administered to a subject to treat a pulmonary disorder or injury.

In some embodiments, the present lung progenitor cells may be used to correct lung-related congenital defects caused by genetic mutations. In particular, mutation affecting human lung development can be corrected using the present lung progenitor cells. In some embodiments, the present lung progenitor cells may be used to generate replacement tissue.

In some embodiments, the present lung progenitor cells may be used to generate replacement lung tissue for lung related disorders.

The CRISPR/Cas system may be used to generate or correct lung disease related gene mutations. The genetically corrected or mutated cell line is then developed into lung progenitor cells.

In one embodiment, the pulmonary disorder or injury is an airway lung disease and/or a distal lung disease. In another embodiment, the pulmonary disorder or injury is pulmonary fibrosis. In another embodiment, the pulmonary disorder or injury is a non-malignant lung disease. In yet another embodiment, the pulmonary disorder or injury is an interstitial lung disease (including congenital interstitial lung diseases, etc.). In certain embodiments, the pulmonary disorder or injury may be a congenital surfactant deficiency.

Non-limiting examples of pulmonary disorders or injuries include, cystic fibrosis; emphysema; chronic obstructive pulmonary disease (COPD); interstitial lung diseases including pulmonary fibrosis, idiopathic pulmonary fibrosis (IPF), Hermansky-Pudlak Syndrome (HPS), hypersensitivity pneumonitis, sarcoidosis, asbestosis, autoimmune-mediated interstitial lung disease; pulmonary hypertension; lung cancer; acute lung injury (adult respiratory distress syndrome); respiratory distress syndrome of prematurity, chronic lung disease of prematurity (bronchopulmonary dysplasia); congenital surfactant deficiencies, including surfactant protein B deficiency, surfactant protein C deficiency, ABCA3 deficiency; ciliopathies; congenital diaphragmatic hernia; pulmonary alveolar proteinosis; pulmonary hypoplasia; lung injury, and combinations thereof. The pulmonary disorder or injury may be HPS-associated interstitial pneumonia (HPSIP).

Pulmonary fibrosis is the formation or development of excess fibrous connective tissue (fibrosis) in the lungs, also described as “scarring of the lung.” Pulmonary fibrosis may be a secondary effect of other diseases. Most of these are classified as interstitial lung diseases. Examples include autoimmune disorders, viral infections or other microscopic injuries to the lung. However, pulmonary fibrosis can also appear without any known cause (termed “idiopathic”), and differs from other forms of fibrosis in that it is not responsive to any immune suppressive treatment.

Alternative approaches to treat diseased lung and airways include the use of tissues reconstituted within decellularized lung matrices. The present lung progenitors may be used to seed a decellularized lung matrix.

The present lung progenitor cells may be used for studying human lung development, modeling lung diseases (e.g., such as RSV infection and fibrosis), testing therapeutic agents, screening drugs, and regenerative medicine.

In some embodiments, the present lung progenitor cells may be used to identify the molecular basis of normal human lung development.

In some embodiments, the present lung progenitor cells may be used to identify the molecular basis of congenital defects affecting human lung development.

Diseases that can be studied using the present lung progenitor cells include genetic diseases, metabolic diseases, pathogenic diseases, inflammatory diseases, etc.

The present lung progenitor cells can be used for culturing of a pathogen and thus can be used as ex vivo infection models. Examples of pathogens that may be cultured using the present lung progenitor cells include viruses, bacteria, prions or fungi that cause disease in its animal host. Thus, the present lung progenitor cells can be used as a disease model that represents an infected state. For example, the present lung progenitor cells may be used to model the morphological features of respiratory syncytial virus (RSV) infection in the human lung (e.g., the distal lung). For example, lung progenitor cells may be generated from RUES2 cells and then infected with RSV.

Lung progenitor cells may be generated from mutated stem cells to study lung diseases including fibrosis, surfactant secretion disease, or cystic fibrosis. The mutated stem cells may have mutations in HPS1, HPS2, HPS3, HPS5, HPS8, ABCA3, and/or telomerase.

To recapitulate fibrosis in vitro, lung progenitor cells may be generated from RUES2 cells carrying a deletion of the HPS1 gene (e.g., engineered using CRISPR/Cas9) which predisposes the cells with high penetrance to IPF.

In certain embodiments, when patient-specific iPSC lines are generated from cystic fibrosis patients, either before or after gene editing to correct the CFTR genetic lesion responsible for the disease, the present lung progenitor cells allow precise interrogation of mutant versus corrected CFTR function through forskolin-induced epithelial sphere swelling assays.

In certain embodiments, iPSCs derived from patients harboring a lung disease related genetic mutation can be corrected, in vitro, using the CRISPR/Cas system to produce a genetically corrected cell line. Production of lung progenitor cells using cells that have been genetically altered for correcting a genetic defect provides a method of testing such genetic alterations for their capacity to correct the disease phenotype.

The term “lung-disease related mutation” as used herein relates to a gene mutation or polymorphism known to cause a lung disease phenotype. For example, certain lung diseases are caused by gene mutations in one or more of the following, non-exhaustive list of genes: HPS1 (gene ID 3257), HPS2 (gene ID 7031; TFF1), HPS3, HPS4 (gene ID 89781), HPS5, HPS8, TERT (e.g., hTERT, gene ID 7015), TERC (e.g., hTERC; gene ID 7012), dyskerin, CFTR (gene ID 1080), DKC1 (gene ID 1736), LYST, SFPTB (gene ID 6439), SFTPC (gene ID 6440), SFTPA1 (gene ID 653509), SFTPA2 (gene ID 729238), MUC5B (gene ID 727897), SHH (gene ID 6469), PTCH (e.g., PTCH1; gene ID 5727), SMO (gene ID 6608), ABCA3 (gene ID 21), PARN, RTEL1, and KIF15.

Cells harboring mutated genes including, but not limited to, those described above, can be subjected to a CRISPR/Cas system. For example, the cells may be subjected to the CRISPR/Cas induced genetic correction at a stage of growth and expansion such at a pluripotent stage. These cells would then be developed into lung progenitor cells as described herein.

The present lung progenitor cells may be used for agent or vaccine screening (e.g., screening for efficacy, toxicity, or other metabolic or physiological activity) or for treatment of (including resistance to treatment of) lung infection, diseases, and injuries. In particular, the present methods, cells, compositions, and kits can be used in assays, e.g., high-throughput assays, e.g., for the discovery of agents to treat lung disorders and injuries.

The present lung progenitor cells may be used to study drug delivery. In some embodiments, the present lung progenitor cells may be used to screen drugs for lung tissue uptake and mechanisms of transport. For example, this can be done in a high throughput manner to screen for the most readily absorbed drugs, and can augment phase 1 clinical trials that are done to study drug lung tissue uptake and lung tissue toxicity. This includes pericellular and intracellular transport mechanisms of small molecules, peptides, metabolites, salts.

The present lung progenitor cells may be used to screen for a test agent. The present screening method may comprise contacting a test agent or a library of agents with the present lung progenitor cells.

In some embodiments, the present lung progenitor cells can be used in vaccine development and/or production. Methods of determining whether a test agent has immunological activity may include testing for immunoglobulin generation, chemokine generation and cytokine generation by the cells.

For acute treatment testing, an agent or vaccine may be applied, e.g., once for several hours. For chronic treatment testing, an agent or vaccine may be applied, e.g., for days to one week. Such testing may be carried out by providing the present lung progenitor cells under suitable conditions (e.g., in a culture medium with oxygenation); applying an agent to be tested (e.g., a drug candidate) to the lung organoid (e.g., by topical or vapor application); and then detecting a physiological response (e.g., damage, scar tissue formation, infection, cell proliferation, burn, cell death, marker release such as histamine release, cytokine release, changes in gene expression, etc.), the presence of such a physiological response indicating said agent or vaccine has therapeutic efficacy, toxicity, or other metabolic or physiological activity if inhaled or otherwise delivered into the lung of a mammalian subject. A control sample of the lung organoid may be maintained under like conditions, to which a control agent (e.g., physiological saline, compound vehicle or carrier) may be applied, so that a comparative result is achieved.

In certain embodiments, the present lung progenitor cells may be used in methods for screening for a test agent that can treat certain condition(s). For example, agents may be screened for preventing or reducing the formation of collagen using the present lung progenitor cells. Agents may be screened for preventing or reducing fibrosis using the present lung progenitor cells. Agents may be screened for preventing or reducing the formation of fibronectin and/or any other extracellular matrix protein, as well as mesenchymal cells (fibroblast, lipofibroblast, myofibroblasts, etc.) using the present lung progenitor cells. Agents may be screened for increasing or decreasing surfactant production using the present lung progenitor cells.

Agents may be screened for treating fibrosis using lung progenitor cells having mutations in one or more genes that are known to cause fibrosis (e.g., HPS1, HPS2, SFTPC and TERC). Cell lines with mutations in HPS and/or LYST may be used as controls, because these mutations affect lysosome-related organelles but are not associated with clinical fibrosis.

Examples of the agents include a small molecule (e.g., a small organic molecule), a protein, a peptide, an antibody or fragments thereof, a nonpeptidic compound, a synthesis compound, a fermentation product, a cell extract, a plant extract, an animal tissue extract, a nucleic acid (e.g., DNA, RNA), a cell culture supernatant, a plasma, or the like. In other embodiments, types of agents include, but are not limited to, peptide analogs including peptides comprising non-naturally occurring amino acids, e.g., D-amino acids, phosphorous analogs of amino acids, such as α-amino phosphoric acids, or amino acids having non-peptide linkages, nucleic acid analogs such as phosphorothioates and PNAs, hormones, antigens, synthetic or naturally occurring drugs, opiates, dopamine, serotonin, catecholamines, thrombin, acetylcholine, prostaglandins, organic molecules, pheromones, adenosine, sucrose, glucose, lactose and galactose.

The present disclosure also provides a kit comprising the present lung progenitor cells, cell population, or pharmaceutical composition. The kit can include a package insert including information concerning cell growth and maintenance, as well as buffers and/or growth factors in the kit.

The present kit may further include containers for suitable administration and a package insert including information concerning the lung progenitor cells, cell population, or pharmaceutical compositions, and dosage forms in the kit. Generally, such information aids researchers, scientists, patients and physicians in using the enclosed lung progenitor cells, cell population, or pharmaceutical compositions effectively and safely. For example, the following information may be supplied in the insert: pharmacokinetics, pharmacodynamics, clinical studies, efficacy parameters, indications and usage, contraindications, warnings, precautions, adverse reactions, overdosage, proper dosage and administration, how supplied, proper storage conditions, references, manufacturer/distributor information and patent information.

As used herein, definitive endoderm (DE) is one of the three germ layers arising after gastrulation that give rise to the intestinal tract, liver, pancreas, stomach and all other organs derived from the AFE, as listed above. DE expresses the markers: FOXA2, FOXA1, cKIT, CXCR4, and EPCAM.

As used herein, “anterior foregut endoderm” (AFE) refers to endoderm that is anterior to the endoderm that gives rise to the most proximal derivatives of the endoderm or primitive gut tube. Anterior foregut endoderm may include, for example, pharyngeal endoderm or lung endoderm and other, more highly differentiated populations of endodermal cells. As embryonic tissues express characteristic sets of molecular markers, the various cell types encompassed by the term “anterior foregut endoderm” may exhibit different expression patterns of molecular markers. Anterior foregut endoderm can give rise to various tissues, e.g., tonsils, tympanic membrane, thyroid, parathyroid glands, thymus, trachea, esophagus, stomach, lung and larynx/pharynx. Anterior foregut endoderm expresses FOXA2, FOXA1, SOX2 and EPCAM and is negative for the distal endoderm marker CDX2.

An organoid may refer to an artificial, in vitro three-dimensional construct created to mimic or resemble the functionality and/or histological structure of an organ or portion thereof. An organoid may refer to a 3-dimensional growth of mammalian cells in culture that retains characteristics of the tissue in vivo, e.g., prolonged tissue expansion with proliferation, multilineage differentiation, recapitulation of cellular and tissue ultrastructure, etc.

A lung bud organoid (LBO) may contain lung epithelial cells (expressing FOXA2, FOXA1, NKX2.1 and EPCAM) and/or mesenchymal progenitors (expressing PDGFRa, CD90, TBX4, and HOXA5). LBOs may be spheroids when generated from anterior foregut endoderm cells in suspension cultures in vitro. LBOs may form between day 15 to day 25 and may include folding structures.

As used herein, a “therapeutically effective” amount is an amount of an agent effective to treat, ameliorate or lessen a symptom or cause of a given pathological condition in a subject suffering therefrom to which the agent is to be administered.

As used herein, a “prophylactically effective” amount is an amount of an agent effective to prevent or to delay the onset of a given pathological condition in a subject to which the substance is to be administered. A prophylactically effective amount refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

“Treating” or “treatment” of a state, disorder or condition includes: (1) preventing or delaying the appearance of clinical symptoms of the state, disorder, or condition developing in a person who may be afflicted with or predisposed to the state, disorder or condition but does not yet experience or display clinical symptoms of the state, disorder or condition; or (2) inhibiting the state, disorder or condition, i.e., arresting, reducing or delaying the development of the disease or a relapse thereof (in case of maintenance treatment) or at least one clinical symptom, sign, or test, thereof; or (3) relieving the disease, i.e., causing regression of the state, disorder or condition or at least one of its clinical or sub-clinical symptoms or signs.

The term “subject” includes any animal, preferably a mammal (e.g., rat, mouse, dog, cat, rabbit) and more preferably a human. Subjects, which may be treated according to the present disclosure, include all animals which may benefit from the present cells, cell populations, pharmaceutical compositions, or methods. Such subjects include mammals. “Patient” or “subject” refers to mammals and includes human and veterinary subjects. Certain veterinary subjects may include avian species.

“Mammalian” and “mammals” as used herein refers to both human subjects (and cell sources) and non-human subjects (and cell sources or types), such as dogs, cats, rats, mice, rabbits, monkeys, etc. (e.g., for veterinary purposes). Mammals include humans (infants, children, adolescents and/or adults), and animals such as dogs and cats, farm animals such as cows, pigs, sheep, horses, goats and the like, and laboratory animals (e.g., rats, mice, guinea pigs, and the like).

As used herein, the phrase “pharmaceutically acceptable” refers to molecular entities and compositions that are “generally regarded as safe”, e.g., that are physiologically tolerable and do not typically produce an allergic or similar untoward reaction, such as gastric upset, dizziness and the like, when administered to a human. The term “pharmaceutically acceptable” may mean approved by a regulatory agency of the Federal or a state government or listed in the U.S. Pharmacopoeia or other generally recognized pharmacopeias for use in animals, and more particularly in humans. Pharmaceutically acceptable excipients, diluents, and carriers for therapeutic use are well known in the pharmaceutical art, and are described, for example, in Remington: The Science and Practice of Pharmacy. Lippincott Williams & Wilkins (A. R. Gennaro edit. 2005). The choice of pharmaceutical excipient, diluent, and carrier can be selected with regard to the intended route of administration and standard pharmaceutical practice.

The term “allogeneic” refers to any material derived from a different animal of the same species as the individual to whom the material is introduced. Two or more individuals are said to be allogeneic to one another.

The term “autologous” refers to any material derived from the same individual to whom it is later to be re-introduced into the same individual.

The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 20%, 10% or 5%.

Standard methods in molecular biology are described Sambrook, Fritsch and Maniatis (1982 & 1989 2nd Edition, 2001 3rd Edition) Molecular Cloning, A Laboratory Manual, Cold Spring Harbor Laboratory Press, Cold Spring Harbor, NY; Sambrook and Russell (2001) Molecular Cloning, 3rd ed., Cold Spring Harbor Laboratory Press, Cold Spring Harbor, NY; Wu (1993) Recombinant DNA, Vol. 217, Academic Press, San Diego, CA). Standard methods also appear in Ausbel, et al. (2001) Current Protocols in Molecular Biology, Vols. 1-4, John Wiley and Sons, Inc. New York, NY, which describes cloning in bacterial cells and DNA mutagenesis (Vol. 1), cloning in mammalian cells and yeast (Vol. 2), glycoconjugates and protein expression (Vol. 3), and bioinformatics (Vol. 4).

The following examples of specific aspects for carrying out the present disclosure are offered for illustrative purposes only, and are not intended to limit the scope of the present disclosure in any way.

### Example 1

We generated two types of expandable cells from hPSC-derived lung organoids (LOs)³³⁻³⁸.

The first are called induced respiratory airway progenitors (iRAPs). The in vivo equivalent of these cells with specific mutation(s) accumulates in IPF as part of the ongoing aberrant repair process. We found that these cells recapitulate all the epithelial hallmarks of IPF, more specifically increased susceptibility to ER stress, spontaneous accumulation of ‘aberrant basaloid cells’, and impaired AT1 differentiation.³⁹ They can therefore be developed into a drug discovery platform. The defects observed in iRAPs mutant for genes involved in IPF justify the pursuit CRT efforts to replace these cells in disease lungs. In certain embodiments, iRAPs are alveolar stem cells that show epithelial hallmarks of IPF when mutant for HPS1. Mutated iRAPs may have increased generation of KRT5-KRT17+ cells at the expense of SCGF3A2+ cells, impaired AT1 differentiation, increased ER stress susceptibility (typically associated with AT2 cells in IPF), ER stress causes AT1 differentiation defect and KRT17 increase.

The second are distal lung epithelial progenitors (DLEPs). DLEPs, which shared features with airway secretory cells types that have been shown to participate in alveolar repair after severe injury in mice,^{25,40} engraft in lung alveoli and promote near complete injury repair after intrabronchial administration to immune-suppressed rats conditioned by prior regional de-epithelialization followed by local irradiation.⁴¹ This approach was well-tolerated. In certain embodiments, DLEPs are cells with airway-like phenotypes that engraft in distal lung of rat model of lung injury and promote repair.

Drugs that correct epithelial dysfunction in IPF can furthermore be complementary to CRT as they would not only improve the function of the distal lung stem cells and AT2 cells affected by IPF but may also promote engraftment and proper differentiation of engrafting cells.

Induced Respiratory Airway Progenitors (iRAPs): A Platform for Drug Screening

iRAPs may be used as a cell-based model for screening of compounds (small molecules, cytokines or antibodies) that directly impact on the epithelial pathogenesis of IPF.

IPF models. A major challenge to developing novel treatments is the availability of well-validated models that fully recapitulate the disease. Several mouse models showed that mutant SP-C proteins are aberrantly folded and lead to toxic gain-of function induction of ER stress and AT2 injury.⁸⁵⁻⁸⁷ Expression of some mutant SP-C proteins during lung development leads to perinatal mortality^{85,88} and expression of other mutations (p.Leu188Gln) after lung development leads to bleomycin-induced fibrosis.⁴⁹ A knock-in mouse model of attenuated expression of the SP-C p.Ile73Thr mutation leads to spontaneous age-related fibrosis when the mutant allele is expressed at low levels, but acute lung injury when the allele is expressed at high levels.⁸⁹ Although many mouse models have no phenotypes unless challenged with bleomycin,⁹⁰ they do indicate increased susceptibility to AT2 injury and a fibrotic response. Mouse models are therefore valuable tools, although higher throughput mechanistic studies, validation of genetic variants and discovery of therapeutic targets are challenging. Rare and common variants have been linked to IPF susceptibility in linkage, candidate gene, GWAS, Mendelian randomization, and next generation sequencing studies in humans.^{3,43,65,77,91,92} Human explanted lung tissue enables genomic, transcriptomic, proteomic, metabolomic and cell-cell interaction studies at the single cell level, and have revealed the accumulation of aBCs and of transitional SCGB3A2⁺ populations.^{26,30,93} Human models are therefore invaluable to verify observations made in mouse and organoid models. A drawback is that most results have been from end-stage lung tissue from a limited number of ILD subtypes. Lung organoids generated from human pluripotent stem cells (hPSCs) offer an inexhaustible source of cells for generating lung lineages of interest for disease modeling, drug screening, or cell-based therapies. The models can be patient-specific (iPSCs) or mutation-specific (gene-edited ESCs). While the reductionist nature of organoids imparts malleability and the possibility of in-depth mechanistic and high-throughput approaches, a limitation of organoid models is the absence of vasculature and immune cells.

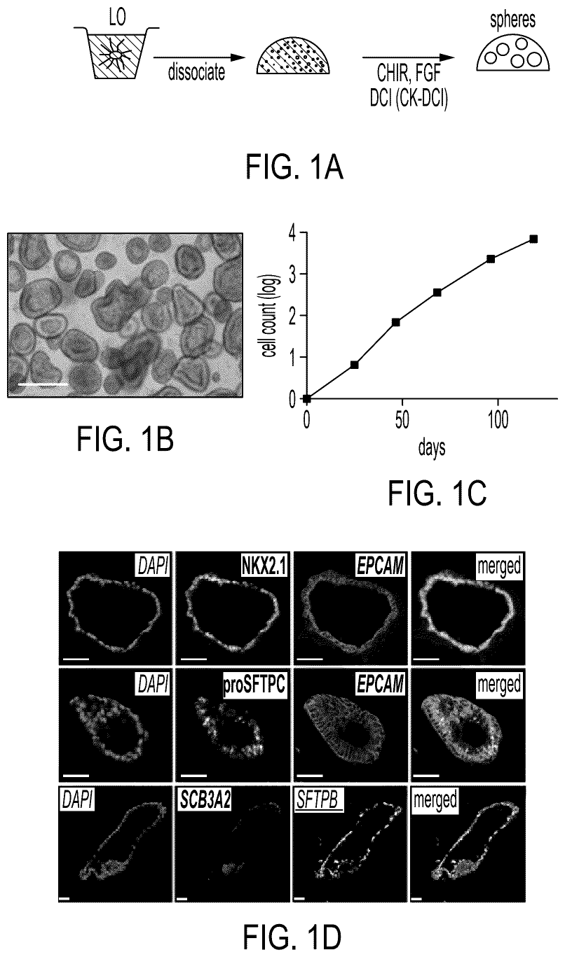

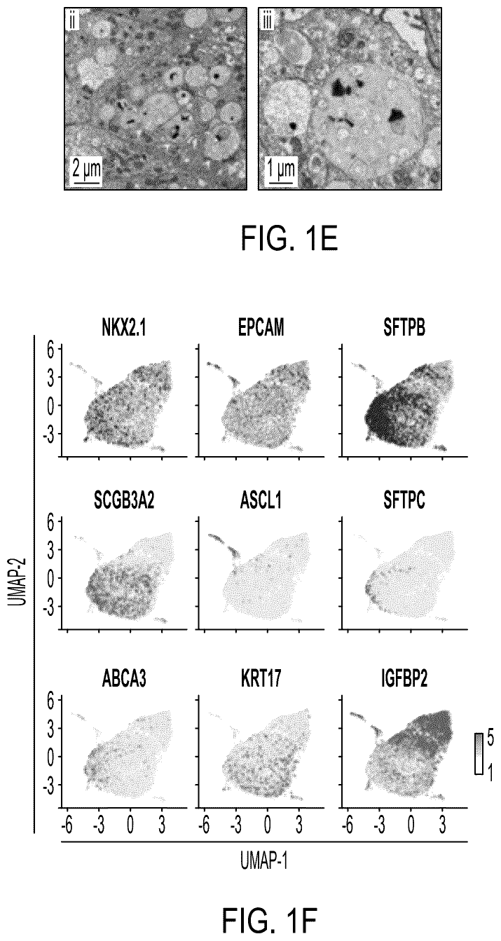

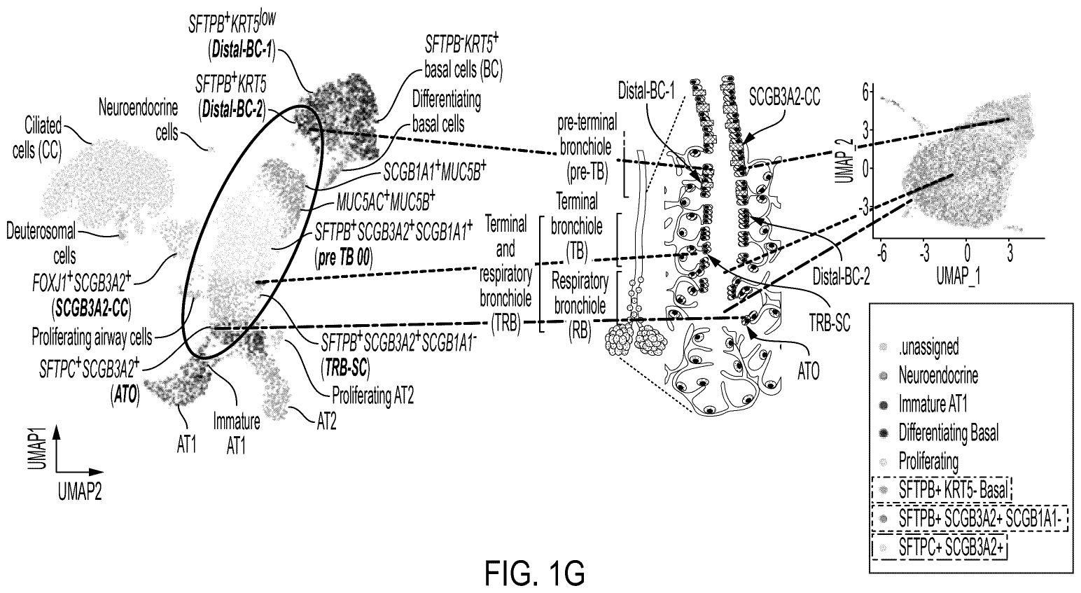

The Induced Respiratory Airway Progenitor (iRAP) Model RA/TRB progenitors. We cultured dissociated cells from LOs in a culture medium supplemented with CHIR, KGF, dexamethasone, cAMP) and IBMX (CK-DCI) (FIG. 1A), previously reported to promote outgrowth of spheres containing AT2 cells (alveolospheres) from hPSC-derived lung progenitors.⁹⁴⁻⁹⁶ Hollow spheres developed that could be serially passaged every ˜3 weeks for up to at least 7 passages with an average expansion of ˜10-fold between each passage (FIG. 1B-1C). Confocal microscopy (FIG. 1D) showed expression of NKX2.1 (lung), EPCAM (epithelial), very sporadic expression of proSFTPC (AT2, one rare organoid shown in FIG. 1d, most are negative), and extensive co-expression of SFTPB and SCGB3A2. The latter expression pattern is very suggestive of RA/TRB cells. Transmission electron microscopy revealed apical presence of multivesicular bodies (MVBs), precursors of lamellar bodies (LBs) in AT2 cells,⁹⁷ (FIG. 1E), a finding consistent with ubiquitous SFTPB expression.^{98,99}

By scRNA the cells were homogenous with respect to the expression of NKX2.1 and EPCAM, and therefore entirely lung epithelium-specified. There was a gradient of SFTPB expression, with a population expressing SCGB3A2 that expresses the most SFTPB (FIG. 1F). Unbiased cell type assignment based on the dataset of Murthy et al.¹⁰⁰ showed that these are TRB-SCs (terminal respiratory bronchiole secretory cells SCGB3A2⁺SFTPB⁺) (FIG. 1G). A small subset of these, and in fact the population that expressed the most SFTPB, also expresses the AT2 markers, SFTPC and ABCA3, corresponding to SCGB3A2⁺SFTPB⁺SFTPC⁺ AT0 cells. Finally, the third major fraction in this continuum is SFTPB^{+/lo}SCGB3A2−. These are classified as distal BCs, BC-like cells present in RA/TRBs that can be low to negative for KRT5 and p63, as was the case here. The only mature cell types are neuroendocrine cells (˜1% of the cells). The other cell identifications we reported concern very rare cells (<1%) in the population that are probably transitions between the aforementioned populations. Taken together, the spheres contain recently identified population associated with RA/TRBs and are therefore called induced respiratory airway progenitors (iRAPs).

Despite using similar culture conditions, iRAPs are remarkably distinct from alveolospheres. Alveopheres as described by the Kotton group are reported to consist of type 2 alveolar epithelial (AT2) cells. They are generated from hPSC-derived lung progenitors after repeated sorting for the lung marker, NKX2.1, and then for the AT2 marker, SFTPC, using reporters.^{94,95} To maintain the alveolospheres as alveolospheres, they need to be re-sorted for the SFTPC reporter, indicating that other cells are overgrowing and displace the AT2 cells. Published scRNAseq of alveolospheres, showed that 17% of the cells are in fact “gastric”. 13% of the cells are called “AT2-like”, though from the UMAPs shown, the basis for this assignment is unclear, since the cells do not even express SFTPB, and early AT2 marker.⁹⁴ The remainder are called “AT2” or “mitotic AT2”. However, the two most specific AT2 markers, ABCA3 and SFTPC, are barely expressed in these clusters. Hence, while alveolopheres may contain AT2 cells, many other uncharacterized cells and a substantial portion of cells that not even lung-specified are present. Another essential difference is that Basil et al. suggest that SCGB3A2⁺ cells (as determined by a reporter gene) were present in airway spheres (generated in low WNT conditions) and converted to AT2 cells when culture conditions were switched to alveolosphere conditions in CK-DCI. As SCGB3A2+ cells disappeared in alveolosphere conditions (CK-DCI), they conclude from these data that SCGB3A2+ RAS (respiratory airway secretory cells in their terminology) unidirectionally differentiate into iAT2 cells.²⁷ In our hands, these same conditions maintain iRAPs, not iAT2 cells. The reason for these major discrepancies is unclear, but the way the cultures are initiated (early 2D cultures with the necessity for repeated sorting for reporters for alveolospheres vs. LOs that may better recapitulate lung development for iRAPs).

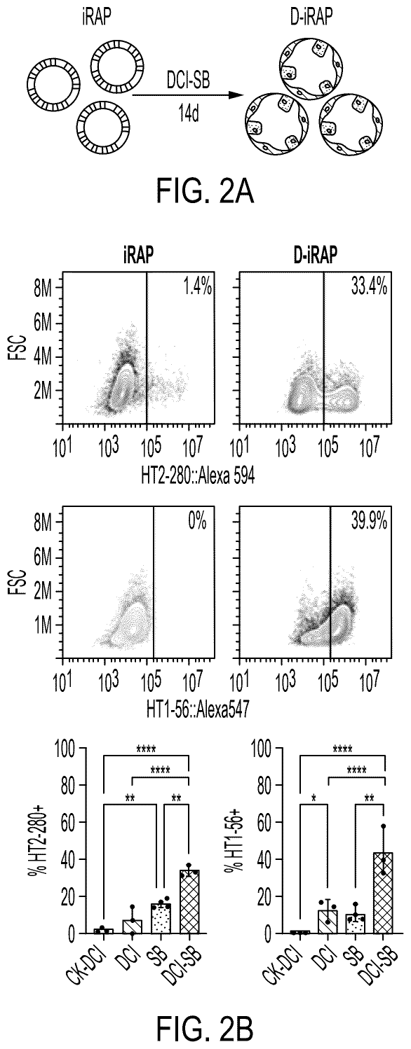

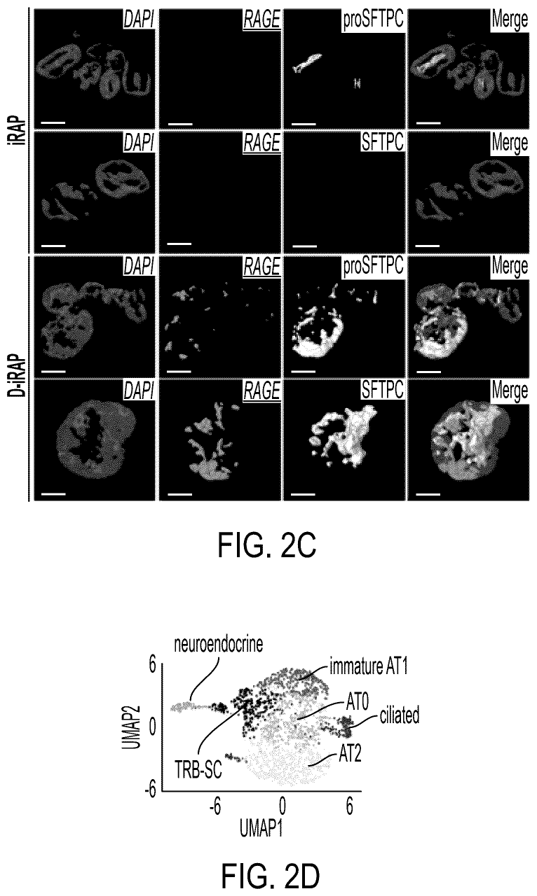

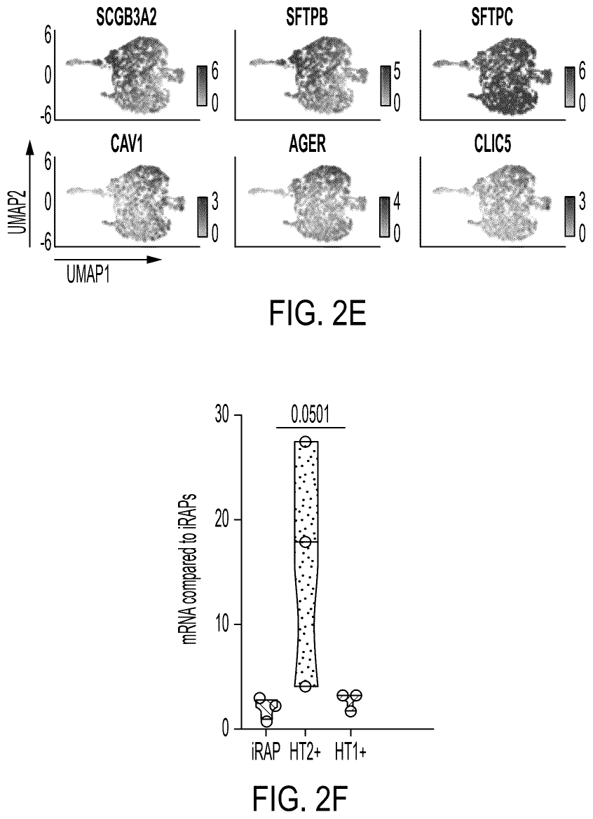

AT0/AT2 cells. In attempt to generate AT2 cells, we sought to mature iRAPs into more distal fates by retraction of CHIR, as CHIR removal was reported to induce AT2 maturation in alveolospheres.^{94,95} CHIR removal, however, led to disintegration of the spheres, decreased proliferation and a modest increase of the HT2-280⁺ fraction which was, however, not corroborated by mRNA expression of the AT2 marker, SFTPC. We did observe however that removing FGFs and CHIR and blocking TGFb induce distal differentiation (FIG. 2A). While the AT1 marker, HT1-56,¹⁰¹ is absent in iRAPs cultured in CK-DCI and expression of the AT2 marker, HT2-280, is low, iRAPs cultured with DCI-SB contained ˜40% HT1-56+ and ˜30% HT2-280⁺ cells (FIG. 2B). The spheres co-expressed SFTPC (AT2) and RAGE (AT1) in a largely non-overlapping pattern. scRNAseq and cell identity assignment using the Human Lung Cell Atlas¹⁰² showed presence of mature AT2 cells, AT0 cells and immature AT1 cells, as well as a small population of neuroendocrine cells and ciliated cells (though we could confirm the presence of the latter as no FOXJ1 was expressed) (FIG. 2D-2E). We therefore call this population AT0/AT2. Sorting for HT2-280⁺ and HT1-56⁺ cells revealed enrichment of SFTPC in the latter (FIG. 2F), confirming their nature as AT0/AT2 cells.

AT1 cells. Expression of AT1 markers and SCGB3A2 overlapped in distalized iRAPs (D-iRAPs), indicating incomplete maturation. AT1 differentiation is regulated by physical forces¹⁰³⁻¹⁰⁵ suggesting that their specification may require adherent 2D culture conditions. Moreover, BMP signaling was previously shown to increase AT1 differentiation efficiency following pneumonectomy in mice,¹⁰⁶ a finding consistent with our observation that the BMP inhibitor, NOGGIN, negatively impacted the specification of alveolar cell types in iRAPs.

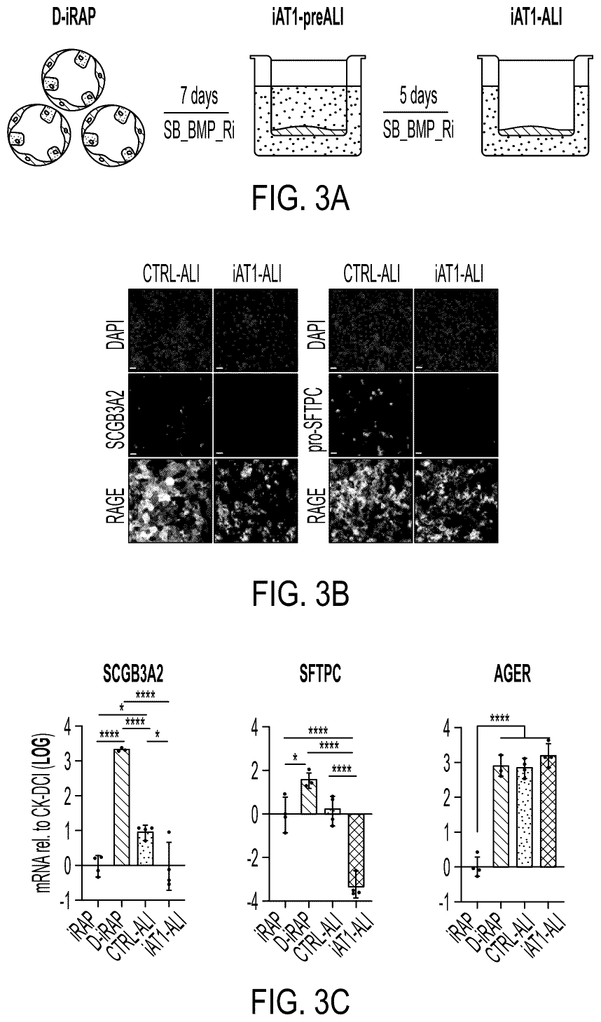

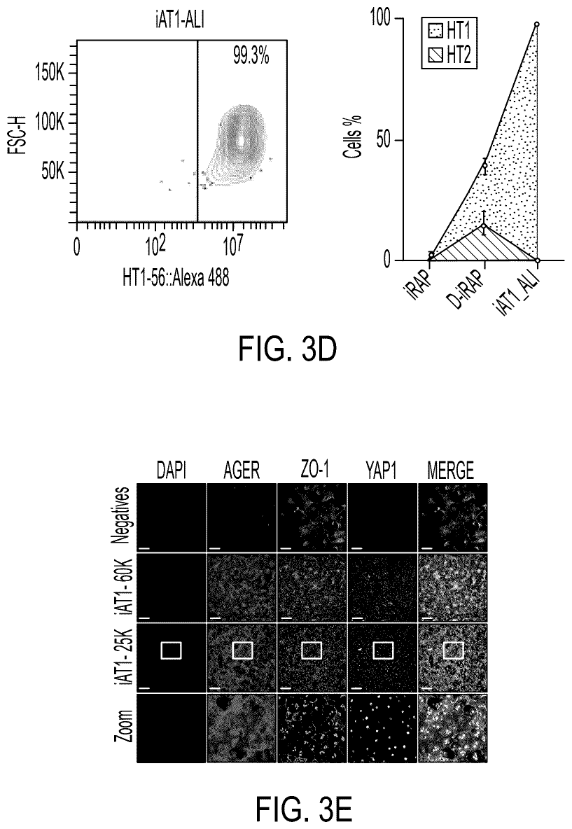

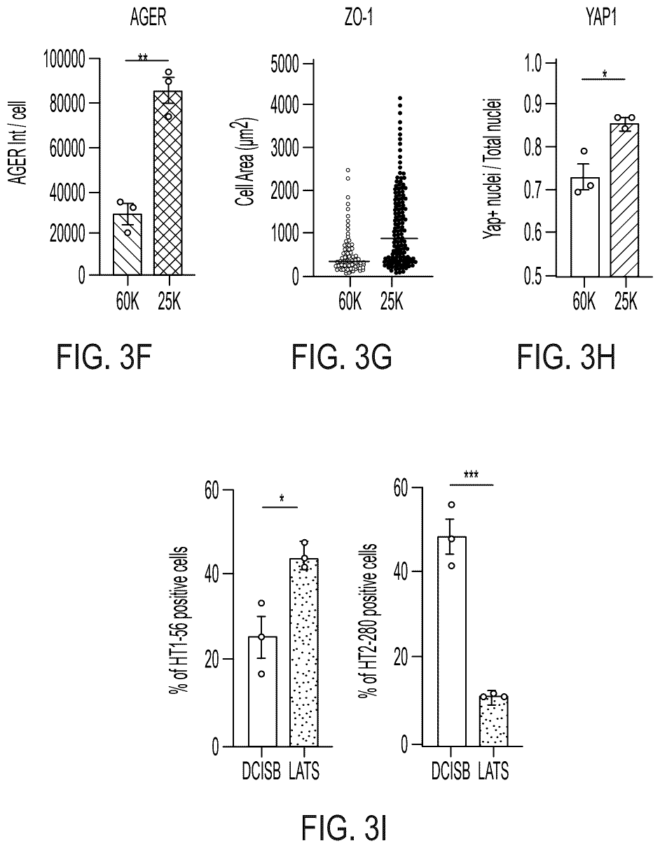

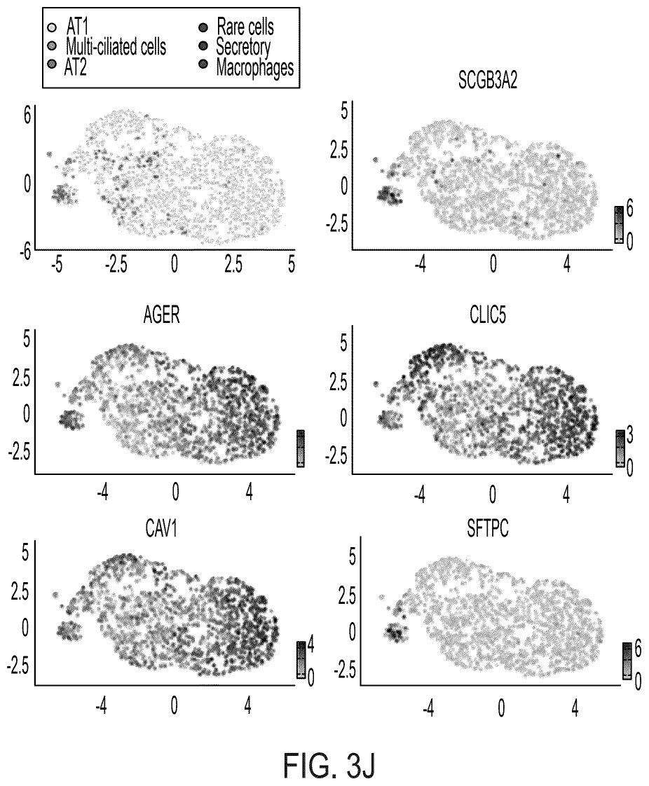

We therefore dissociated D-iRAP spheres and plated the cells on Matrigel-coated plates and subsequently in air-liquid interphase (ALI) culture (FIG. 3A). In 2D culture with BMP4 alone did not allow cells to adhere (not shown), but high expression of AGER mRNA was maintained in the presence DCI-SB, SB only and BMP4-SB (FIG. 3B). Interestingly, BMP4-SB also downregulated SCGB3A2 and SFTPC mRNA indicative of AT1 at the expense of AT2 maturation (FIG. 3B). In subsequent ALI conditions, this expression pattern was maintained: cells cultured in BMP4-SB maintained confluency, almost universally expressed AGER but lost expression of SFTPC and SCGB3A2 protein (FIG. 3C), whereas near 100% of the cells expressed HT1-56 (FIG. 3D). Furthermore, most cells showed nuclear localization of YAP (FIG. 3E), a feature of AT1 cells.¹⁰⁷ We also cultured iAT1 cells in lower density, where the cells still become confluent. At lower density, size and the expression of AT1 markers (amount of RAGE per cell as delineated by ZO1 membrane expression and fraction of YAP+ nuclei) increases (FIG. 3F-3H). This finding is consistent with a role for physical forces in the induction of AT1 maturation.¹⁰⁵ Finally, we added an inhibitor of LATS, which promotes YAP nuclear localization and transcriptional activity.¹⁰⁸ LATSi inhibited AT2 and promoted AT1 generation (FIG. 3I). This is exactly as expected from in vivo data in the mouse, where conditional deletion of YAP impairs AT1 differentiation after injury.^{107,109,110} scRNAseq analysis showed that >90% of the cells were classified as AT1 cells by Azimuth (based on Human Lung Cell Atlas)¹⁰² (FIG. 3J). A minor fraction co-expressed SCGB3A2, AT1 markers and SFTPC, indicative of AT0 identity (although Azimuth spuriously classified the cells as ‘secretory’ and ‘macrophages’). We conclude that AT1 maturation requires inhibition of TGF-b and activation of BMP signaling in both submerged and ALI 2D conditions. We call these cells induced AT1 (iAT1) cells.

Modeling epithelial defects in IPF. Hermansky-Pudlak Syndrome is caused by abnormal trafficking of endolysosome-related organelles due to mutation in one of 11 HPS genes and characterized by pigmentation abnormalities and platelet dysfunction, as both melanosomes and platelet delta granules are ELROs.¹¹¹⁻¹¹⁵ Select genotypes are associated with other abnormalities, including colitis, schizophrenia, immunodeficiency, and hemophagocytic lymphohistiocytosis indicating differential requirement of individual HPS genes for the trafficking of specific ELROs such as synaptic vesicles, lytic granules of lymphocytes, or dense core vesicles of intestinal Paneth cells.¹¹⁵ The HPS genes belong to four protein complexes: BLOC1, BLOC2, BLOC3 and AP3. HPS1 and 4 form BLOC3, HPS2 is part of AP3.¹¹¹⁻¹¹⁵ HPS1, 2 and 4 show a high (likely 100%) incidence of HPSIP,¹¹³ an entity considered clinically indistinguishable from IPF. As IPF is associated with dysfunction of AT2 cells, ¹¹⁶I HPSIP is believed to be related to abnormal biogenesis and trafficking of LBs, which are also ELROs. We showed previously that mutation of HPS1 induced extensive fibrosis in hPSC-derived LOs.^{34,38} We therefore examined whether iRAPs have intrinsic defect associated with IPF.

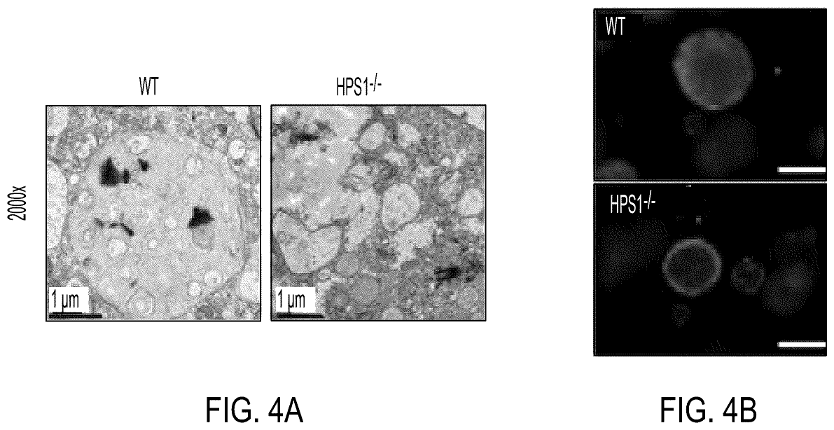

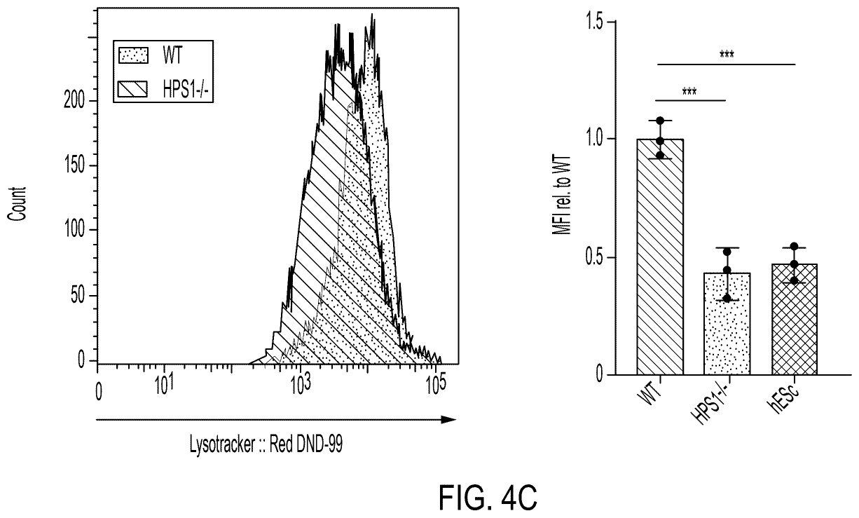

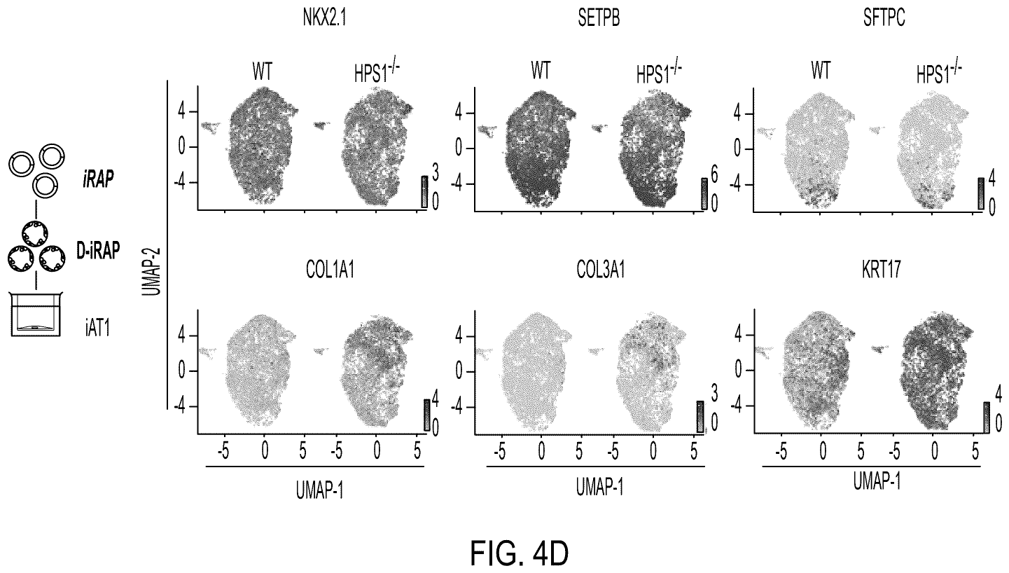

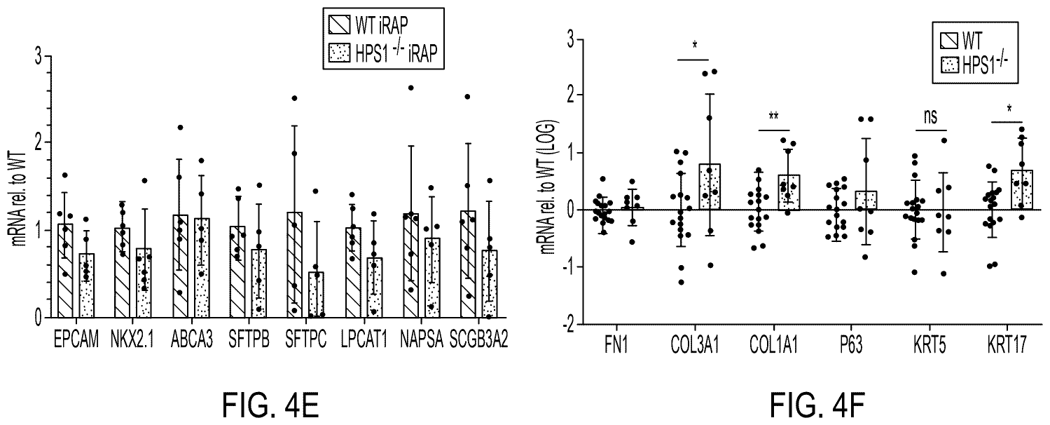

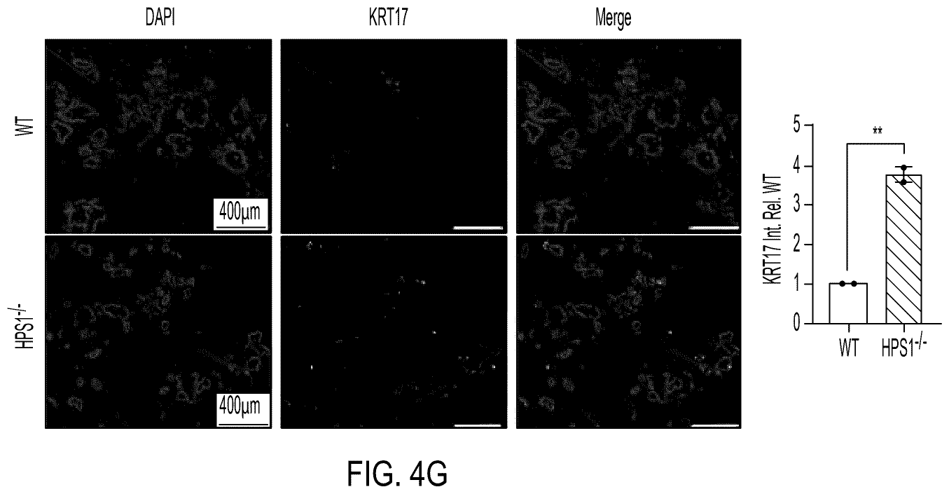

As HPS genes affect lamellar bodies, we first performed transmission electron microscopy (TEM). TEM of WT and HPS1^{-/-} iRAPs. (FIG. 4A) showed multivesicular bodies containing ‘onion-ring’ structures WT the appeared empty in HSP1^{-/-} iRAPs, thus clearly showing a defect in the (still immature) lamellar bodies. Consistent with these findings, Lysotracker Red fluorescence was reduced in HPS1^{-/-} compared to WT iRAPs (FIG. 4B-4C), indicative of dysfunction of the lysosomal/endolysosomal compartment (which includes LBs). HPS1^{-/-} iRAPs therefore showed abnormalities in the LB/MVB system.

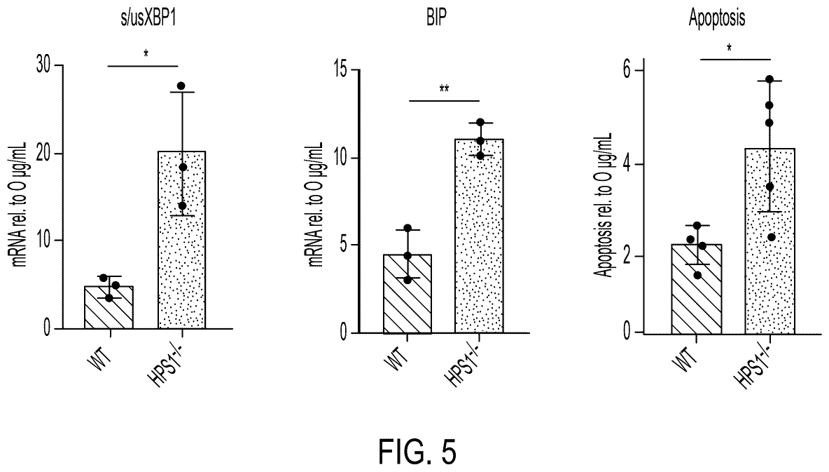

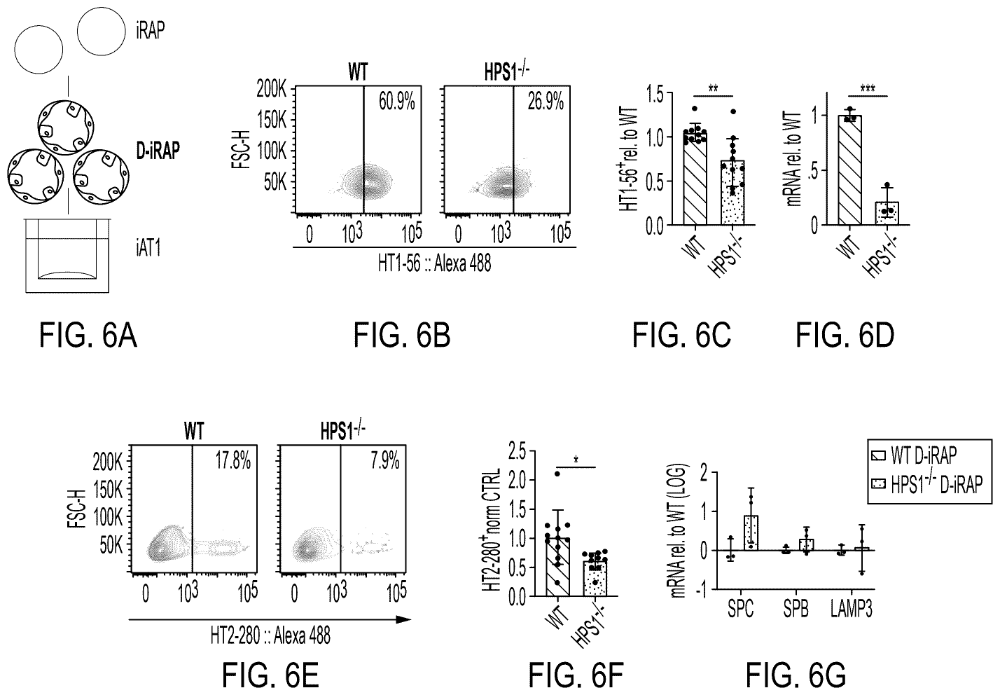

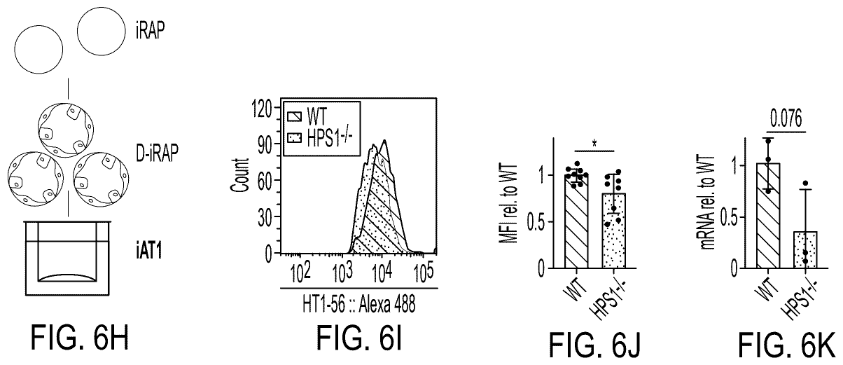

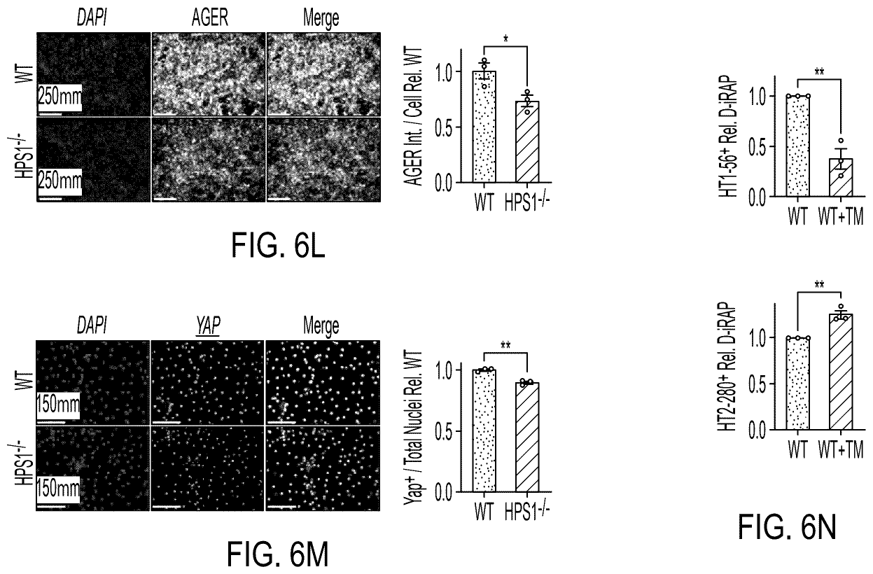

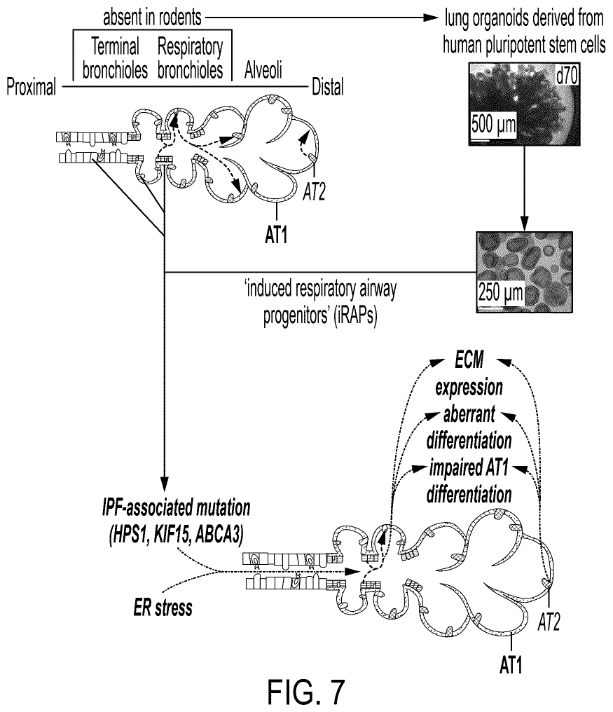

Next, we examined the presence of the three epithelial hallmarks of IPF: (a) generation of aberrant basaloid cells (aBC),^{30,31} (b) increased ER stress,¹¹⁶ and (c) impaired AT1 differentiation. 30,31
a. Accumulation of aberrant basaloid cells (FIGS. 4A-4G). scRNA-seq of WT and HSP1^{-/-} iRAPs showed similar presence of NKX2.1 and SFTPB with a small fraction of SFTPC⁺ cells in both genotypes (FIG. 4D), indicating normal development of the spheres. Similar expression of distal lung markers was also confirmed by RT-qPCR (FIG. 4E). HPS1^{-/-} iRAPs, however, contained more cells expressing KRT17, COL1A1 and COL3A1 (FIG. 4D), which was confirmed by RT-qPCR (FIG. 4F), a phenotype consistent with aBC-like cells. Consistent with our scRNAseq data, no KRT5+ cells were found but cells expressing KRT17 were identified, though at a lower frequency than expected from the scRNAseq data, suggesting that KRT17 is mostly expressed at the mRNA, but not at the protein level at this stage. In HSP1^{-/-} iRAPs, however, KRT17 expression was increased fourfold compared to WT (FIG. 4G), confirming scRNAseq (FIG. 4D) and RT-qPCR (FIG. 4F) data. Mutant iRAPs therefore spontaneously generated aberrant basaloid cells.
b. Increased susceptibility to ER stress (FIG. 5). Given the potential role of ER stress in IPF,¹¹⁶ we assessed the unfolded protein response of the ER (UPR^{ER}). Addition of tunicamycin, an inhibitor of glycosylation that induces ER stress, increased XBP1 splicing (FIG. 5, left panel), BIP expression (FIG. 5, middle panel), and apoptosis (FIG. 5, right panel) in HPS1^{-/-} compared to WT iRAPs, indicative of increased ER stress susceptibility.
c. Impaired differentiation into AT1 cells (FIGS. 6A-6N). At the D-iRAP stage (FIG. 6A), differentiation of HPS1^{-/-} cells was impaired especially into the AT1 lineage as evidenced by a reduced fraction of HT1-56⁺ cells (FIG. 6B-6C) and a sharp reduction in AGER expression (FIG. 6D). In the AT2 lineage, a mild decrease in HT2-280⁺ cells was noted (FIG. 6E-6F), but expression of other AT2 markers, LAMP3, SFTPB and SFTPC, was not significantly affected (FIG. 6G). At the iAT1 stage (FIG. 6H), HPS^{-/-} cells, while uniformly expressing HT1-56 (FIG. 6I), showed a decrease in HT1-56 mean fluorescence intensity (FIG. 6J) and a striking reduction in AGER mRNA (FIG. 6K) and RAGE protein (FIG. 6L) compared to WT, indicative of a differentiation defect. Furthermore, we found reduced fraction of YAP+ nuclei (FIG. 6M). Given the role of both AT1 depletion and ER stress in IPF, we next examined whether these features are linked by assessing the effect of ER stress on AT1 differentiation in WT cells. Induction of ER stress by low-dose tunicamycin (which induces ER stress) in D-iRAPs specifically inhibited AT1, and even enhanced AT2 commitment in D-iRAPs (FIG. 6N), thus quite elegantly linking these AT1 defects and ER stress.
d. Other mutations associated with IPF. We performed similar experiments with iRAPs generated from ESC with mutation in KIF15 (associated with familial IPF)³ and ABCA (associated with both IPF and chILD).^{62,63} Similar data (though less pronounced of ABCA3^{-/-} iRAPs) were obtained with all mutants.
Conclusions (FIG. 7). hPSC-derived iRAPs consist of a continuum of regenerative populations identified in human terminal airways, can undergo self-renewal or be differentiated into AT1 and AT2 cells in defined conditions, and show intrinsic abnormalities that are hallmarks of IPF when mutant for HPS1, KIF15 and ABCA3, thus modeling aberrant regeneration in IPF.

Together our observations suggest that a differentiation defect into the AT1 lineage, at least among others caused by ER stress, is associated with generation of likely profibrotic aBCs, which may represent the default pathway when AT1 cell differentiation is impaired. IPF is therefore at least in part a disease of aberrant differentiation by distal lung stem cells that show intrinsic defects and that is modeled by iRAPs (FIG. 7).

The iRAP model provides a tool to screen for drugs that directly impact on epithelial pathogenesis of IPF. There is currently no model to examine epithelial pathogenesis of IPF and to potentially screen for drugs that direct impact on the key pathogenic mechanism of this intractable disease.

In certain embodiments, the present cell therapy method for IPF or chILD replaces the distal lung stem cell compartment rather than just AT2 cells.

Drug Screen Using iRAPs

The goal of this task is to build the iRAP model into a drug screening tool for IPF and to use the cells for rational, hypothesis-driven drug target discovery. Literature^{117,104,103,118,119} and our own data³⁹ indicate that impairment of AT1 differentiation, leading to depletion of what is the considered the primary cell of the lung, and the reciprocal default generation of extracellular matrix-producing KRT5⁻KRT17⁺ aberrant basaloid (aBC) cells^{31,120} from distal lung progenitors³⁹ may be the root cause of progressive fibrosis. This is the final common pathway of the multiple injuries, genetic and environmental (including ER stress), that are associated with IPF. The drug discovery platform may have a read-out that reflects efficiency of AT1 differentiation at the expense of aBC generation.

We will generate iRAPs mutant for at least two IPF-associated mutations that contain a reporter for RAGE, an AT1 marker, and KRT17, which marks the aBC population, and validate these lines. Reduced soluble RAGE (sRAGE) in the peripheral blood is a potential biomarker for IPF severity and progression, mostly likely because sRAGE levels reflect the depletion of AT1 cells in the lung. ¹¹⁸

We will insert a mScarlet reporter at the 3′ end of the AGER gene (which encodes the RAGE protein) separated by a P2A sequence, and a ZsGreen reporter 3′ of the KRT17 gene in the RUES2 hESC line. We will next delete in these reporters HSP1 and KIF15, genes mutant in familial IPF^{3,113} and mutation of which in iRAPs reproduced epithelial hallmarks of IPF, so that we obtain WT, HSP1^{-/-} and KIF15^{-/-} co-isogeneic knockout lines with identical reporters. This approach eliminates bias caused by genetic background, which may affect the phenotype of knockouts. The faithfulness of the reporters will be assessed by correlating their expression with that of the reported genes throughout the generation and differentiation of iRAPs. A second validation relies on the fact that mutant lines should contain more ZsGreen+ cells in renewing iRAP conditions, and show reduced mScarlet induction after differentiation into AT1 cells compared to the WT. Further validation of the mScarlet/RAGE reporter is based on the finding that BMP agonism and TGF-b antagonism are required to generated AT1 cells.³⁹ Hence, in the presence of BMP inhibition (using NOGGIN), mScarlet⁺ cells should not arise.

A second, complementary approach is the in-depth analysis of both mutants in renewing and differentiating conditions using scRNAseq, RNAseq and proteomics. The latter is important given the fact that in both mutants, we observed increased susceptibility to ER stress, a feature of IPF, which regulates translation. We furthermore observed that inflicting ER stress in WT iRAPs reproduced the IPF phenotypes present in mutant iRAPs. These studies will reveal pathways that may be specifically targeted. Our current findings in HPS1^{-/-} iRAPs, for example, suggest a role for endolysosomal trafficking.

We will conduct directed differentiation of hPSCs into lung organoids and use CRISPR-Cas9 technology to generate the mutant hPSC lines. We will then initiate screening for small molecules that correct IPF-associated defects and/or enhance proper differentiation. Such compounds may aid in the development of optimal culture or engraftment conditions for DLEPs and may be adjuvants in CRT.

### Distal Lung Epithelial Progenitors: Engrafting Cells

As IPF and chILD are in essence epithelial diseases of the distal lung, this study is to develop epithelial CRT as an innovative curative treatment through generation and validation of engrafting distal lung progenitor cells and through the development of novel preclinical models in swine genetically modified to develop chILD and/or IPF. The models will also be used to study the pathogenesis of IPF and chILD and to test other novel therapeutic approaches.

Populations involved in lung regeneration. As type 1 alveolar epithelial (AT1) cells are the primary cell of the respiratory system and all other cells support the function of AT1 cells, gas exchange, lung regeneration is geared towards preserving or regenerating AT1 function.¹²¹ The best characterized and most studied mechanism in the mouse is transition of a fraction of surfactant-producing AT2 cells to AT1 cells through transitional intermediates,^{117,122-125} although AT1-to-AT2 plasticity exists in the neonatal mouse lung¹²⁶ and after partial pneumonectomy in the adult mouse lung.

Airway-derived cells can also contribute to alveolar regeneration after severe injury. A ‘bronchioalveolar stem cell’ at the junction between bronchioli and alveoli that expresses both SCGBlA1, a secretory cell marker, and SFTPC, an AT2 marker, may contribute to alveolar regeneration.^{127,128} After severe injury, a population of p63⁺KRT5⁺ cells resembling airway basal cells (BCs) migrates distally as ‘KRT5-pods’, and are viewed by some investigators as ‘dysplastic’.^{21,22} We are less inclined to this view, as dysplasia classically implies pre-malignancy, for which there is no evidence, and as conditional deletion of these cells (using a Krt6-Cre) impaired regeneration of ‘nets’ of AT1 cells after injury, indicating a direct or indirect role in repair however.²² Similar ‘pods’ are observed in human lungs after severe injury.¹²⁹ These cells originate at least in part from a heterogeneous population of lineage-negative epithelial progenitors (LNEPs),²⁴ that also includes a population lineage traced by the secretory cell (SC) marker, Scgb1a1 (but not expressing this marker).²⁵ Moreover, multiple publications have used lineage tracing to show that SCs^{28,29} or subpopulations thereof expressing a variant secretory marker, Upk3a,^{130,131} can convert to alveolar cells. Lineage tracing evidence in the mouse furthermore showed that the aforementioned transitional intermediates between that AT2 and AT1 cells can be derived from airway SCs after severe injury, providing perhaps the most compelling evidence for an airway-derived input into the transitional cell population pool that arises in the distal lung after severe injury.²³

Single cell transcriptomic studies have indicated AT2-to-AT1 plasticity in humans.^{100,120,121,132} A role for secretory-like (SL) cells has been revealed in humans as well, however. Cells co-expressing the secretory marker, SCGB3A2, and varying levels of AT1 and AT2 markers have been identified by scRNAseq of normal and fibrotic human lungs. These were interpreted as transitional between AT2 and AT1 states. In contrast to mice, primates and ferrets have ‘terminal respiratory bronchioles’ or ‘respiratory airways’ (RA/TRBs) into which multiple alveoli coalesce. In these structures, SCGB3A2⁺ cells were identified that, based on organoid studies and trajectory analysis of scRNAseq data, have alveolar potential and were more abundant after lung injury, and therefore likely play a role in injury repair.^{27,133,134} A subpopulation co-expressing SCGB3A2 and the mature AT2 marker, SFTPC, has been called “AT0” cells and is likely similar to the aforementioned transitional cells. These SCGB3A2-expressing cell types are absent in mice. These cells are modeled by iRAPs derived from hPSCs.

Therefore, AT2-to-AT1 transition may be an important alveolar repair mechanism, but those subsets of cells with SL phenotypes participate in distal lung regeneration as well, most likely more so after severe injury. Distal lung regeneration in humans may differ from mice, but involvement of transitional cells with SL phenotypes appears to be a commonality. Participation of SL cells in injury repair may be viewed as a reserve mechanism when AT2 cells are depleted, similar to the participation of submucosal gland myoepithelial cells to airway regeneration after severe and repeated injury where airway BCs are depleted.

Engraftment models and engrafting cell populations. Engrafting cells can be derived from adult or fetal lungs, or from human pluripotent stem cells (hPSCs). Mouse fetal distal tip progenitors, isolated using a SOX9-GFP reporter, expanded mouse AT2 cells,¹³⁵ mouse pluripotent stem cell (PSC)-derived AT2 cells and adult mouse epithelial organoid cells,¹³⁶ and LNEPs have been shown to engraft to some extent in mouse lungs treated with bleomycin. PSCs, comprising embryonic stem cells (ESCs), derived from the inner cell mass of the blastocyst, and induced pluripotent stem cells (iPSCs), generated by reprogramming of somatic cells¹³⁷ can undergo unlimited expansion in vitro and are therefore widely available and accessible. A major application for engraftment models is the functional assessment of human lung populations generated from hPSCs.

Only few studies on engraftment in NSG lungs have been reported, however. Human hPSC-derived BCs (iBCs) engraft large airways for polidocanol-treated NSG mice, although engraftment is more efficient in mouse-to-mouse transplantation.¹³⁹ PSC-derived iAT2 cells, however, have only been shown to engraft in mouse-to-mouse transplants.¹⁴⁰ Nikolic et al. could achieve engraftment of bleomycin-treated NSG mice with expanded putative human fetal lung DTPs, but did not monitor differentiation or function.¹⁴¹ The Spence group reported the generation of DTPs from organoids generated from hPSCs that showed some engraftment potential in the airways, but not distal lung, of naphthalene-treated NSG mice.^{142,143} Rosen et al. used naphthalene combined with irradiation to engraft canalicular stage fetal lung cells and adult lung cells through, quite remarkably, intravenous administration. Furthermore, they observed engraftment of proximal and distal cells, as well as mesenchymal and endothelial cells, and could reproduce these data with human cells in NSG mice.

### Distal Lung Epithelial Progenitors (DLEPs)

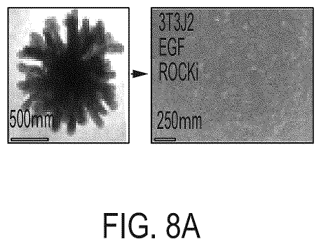

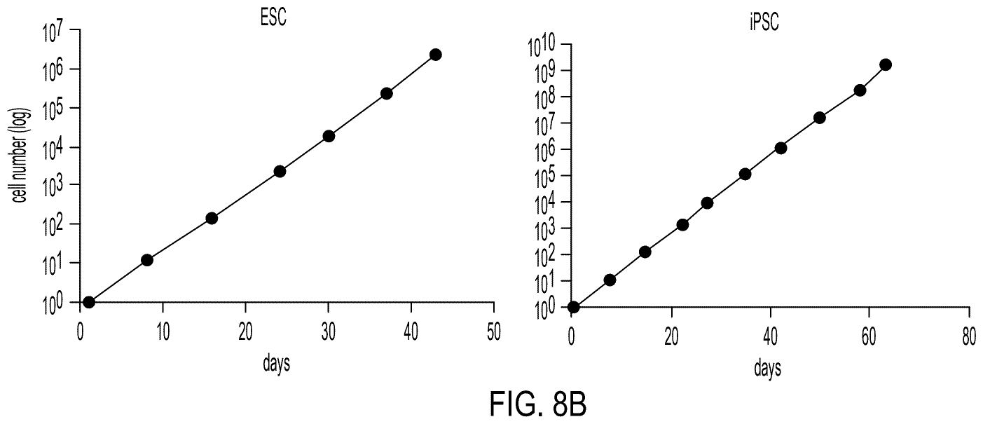

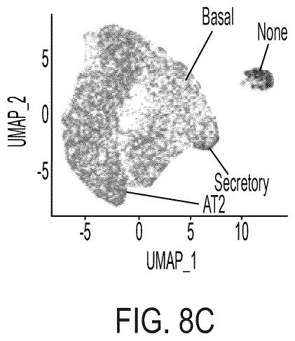

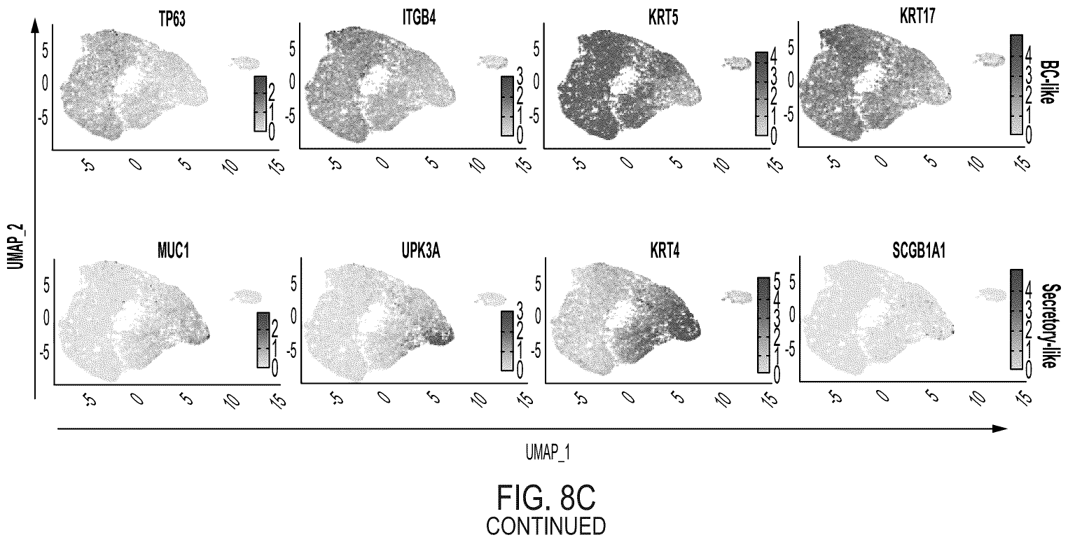

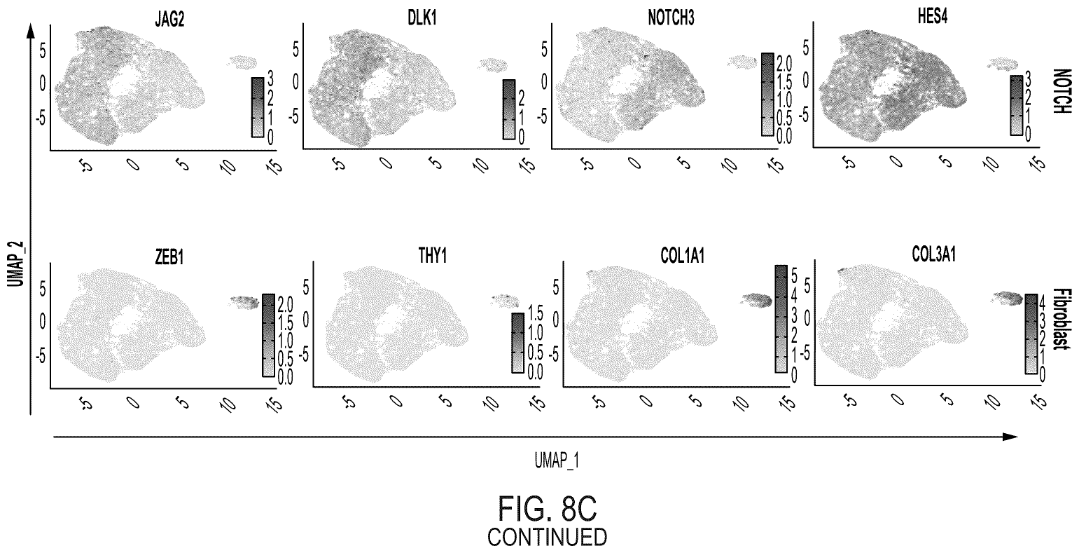

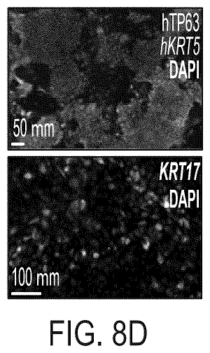

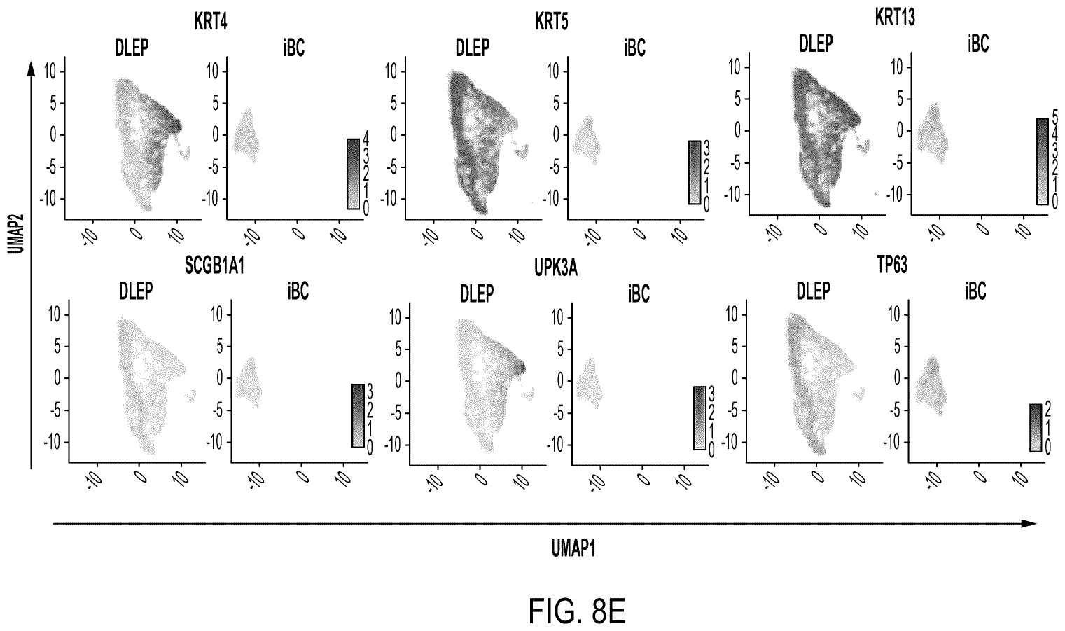

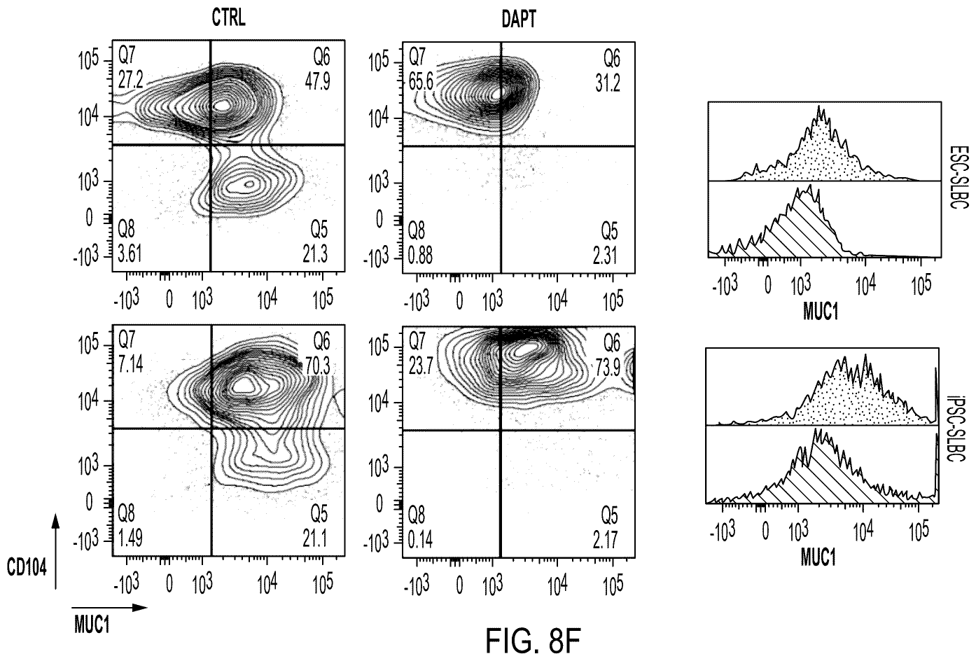

We developed a directed differentiation strategy into 3D lung organoids (LOs) that undergo a process similar to branching morphogenesis by recapitulating lung development through successive specification from definitive endoderm to lung (FIG. 8A).^{35,36,144-146} Dissociated LOs grown in the presence of 3T3-J2 feeders, ROCK inhibitor and EGF (FIG. 8A), conditions that expand epithelial cells from a variety of organs,¹⁴⁷ yielded lines that expanded exponentially and continuously (FIG. 8B), and maintained normal karyotype. Cell type assignment based on the Human Lung Cell Atlas (HLCA) using Azimuth indicated the presence of secretory (SC), basal (BC) and AT2 cells (FIG. 8C). The identification of AT2 cells was surprising, as AT2-specific markers (SFTPC, SFTPB, ABCA3, NAPSA) were not detected by immunofluorescence (IF), RT-qPCR or scRNAseq. These data may suggest that a fraction of the cells may be precursors for AT2 cells, however. In addition, a small population (˜2%) was assigned as fibroblast, T and dendritic cells using HLCA. Inspection of expressed genes in these did not identify any hematopoietic lineage-defining markers such CD45. By virtue of expression of Thy1, Zeb1 and collagens, we classify the cells as fibroblasts (FIG. 8C). In the organoids we derived the cells from a small population of pulmonary mesenchyme is consistently present as well.

Supervised analysis allowed subsetting into two major clusters based on expression of NOTCH ligands and receptors: BC-like and secretory-like (FIG. 8C). Because of their potential to engraft in the distal lung of conditioned rats (see below), we call the cells ‘distal lung epithelial cells’ (DLEPs). The secretory-like cluster (also classified as ‘secretory’ by Azimuth) expressed MUC1, NOTCH3, UPK3A, KRT4, and KRT13 (feature plots shown in FIG. 8C). KRT4 and KRT13 are also expressed in airway ‘hillocks’ in the mouse and are believed to an intermediate stage between BCs and secretory cells.¹⁴⁸ In the mouse, UPK3A is expressed in rare variant club cells that reside near neuroendocrine bodies,^{130,149} and play a role in airway regeneration. The expression of NOTCH ligands is consistent with the requirement for NOTCH signaling in the secretory lineage.¹⁵⁰ The classical mature secretory marker, SCGB1A1, was absent, however, indicating that they are “variant” secretory cells. The second and major population (that was partially assigned to the AT2 lineage and the BC lineage) expressed p63, KRT5, KRT17 and ITGB4 (FIG. 8C) as well as the NOTCH ligands, JAG2 and DLK2 (FIG. 8C), suggesting mutual regulation of both fates. Expression of KRT5, KRT17 and p63 were confirmed by IF (FIG. 8D).

We next matched the scRNAseq data with the hPSC-derived induced basal cells (iBCs), reported by the Kotton lab, that engraft airways of NSG mice after de-epithelialization with polidocanol.¹³⁹ In contrast to iBCs, DLEPs expressed UPK3A+, KRT4 and KRT13, and KRT5 (FIG. 8E). These data support our conclusion that DLEPs contain basal-like cells and variant SCs but are distinct from classical SCs and BCs. As Upka3a mRNA expressing cells (the protein is not detected in the mouse) have been shown in lineage tracing studies to contribute to alveolar repair after bleomycin injury, this is an appropriate population to test in engraftment studies.

Staining for CD104 and MUC1 showed two populations: MUC1^{hi}CD104^{lo} (secretory-like) and MUC1^{lo}CD104^{hi} (BC-like) by flow cytometry (FIG. 8F), consistent with the scRNAseq data. In the presence of the γ-secretase inhibitor, DAPT, MUC1^{hi}CD104^{lo} cells disappeared, a finding consistent with expression of NOTCH in these cells, and the culture could not be passaged anymore. These data suggest that MUC1^{hi}CD104^{lo} secretory-like cells are the progenitors among DLEPs and are strictly NOTCH-dependent for expansion.

### Development of a Lung Injury and Engraftment Model

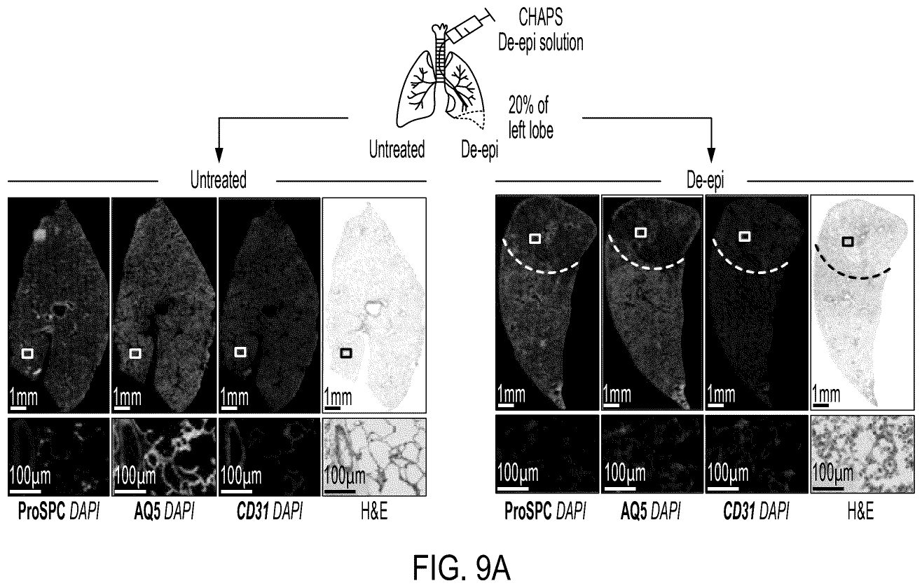

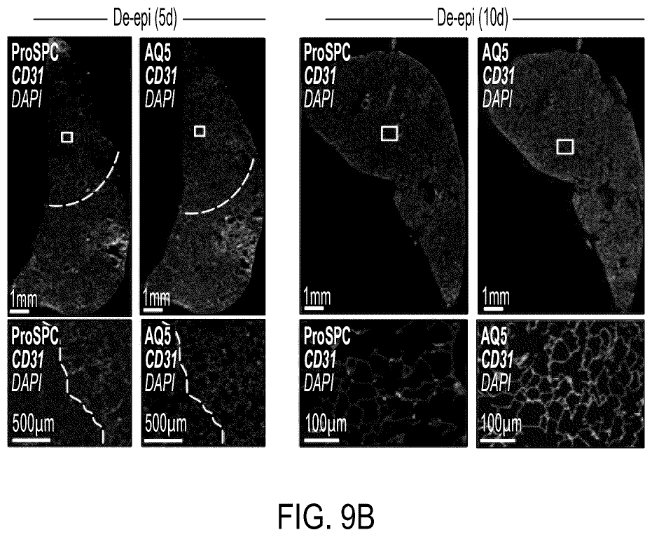

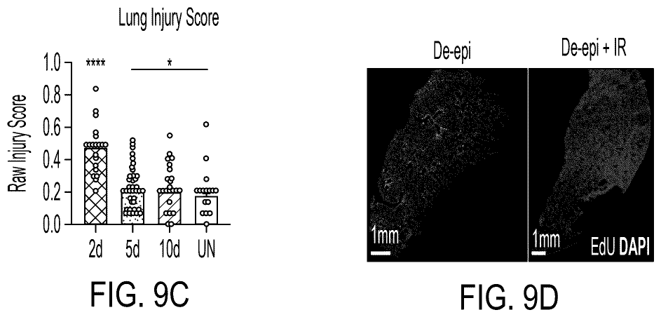

We developed a model based on locoregional de-epithelialization in a larger animal, the rat, using CHAPS, a mild, non-denaturing, zwitterionic detergent that was instilled twice using engineered cannula in the lower left lung of an anesthetized, ventilated and monitored rat, with re-aspiration after 30 mins (FIG. 9A). Mortality was <5% during the anesthesia and intubation, but none of the surviving animals showed signs of respiratory distress or blood abnormalities at 48-hours. At that time, treated lung regions showed loss of epithelial cells [AT1 (Aq5) and AT2 cells (pro-SPC)] and preservation of endothelial cells (CD31) by immunostaining (FIG. 9A). After 5 days, partial recovery of alveolar epithelium with increased expression of Aqp5 and pro-SPC in the treated regions was observed. At day 10, the alveolar epithelium was fully reconstituted (FIG. 9B). The lung injury score (LIS), assessed according to ATS guidelines¹⁵¹ was significantly elevated in the lower left lung (treated) compared to the right lung (untreated) within the same animal (FIG. 9C). EdU⁺ proliferating cells increased in and around the treated region at d2 and was abolished by local irradiation (FIG. 9D).

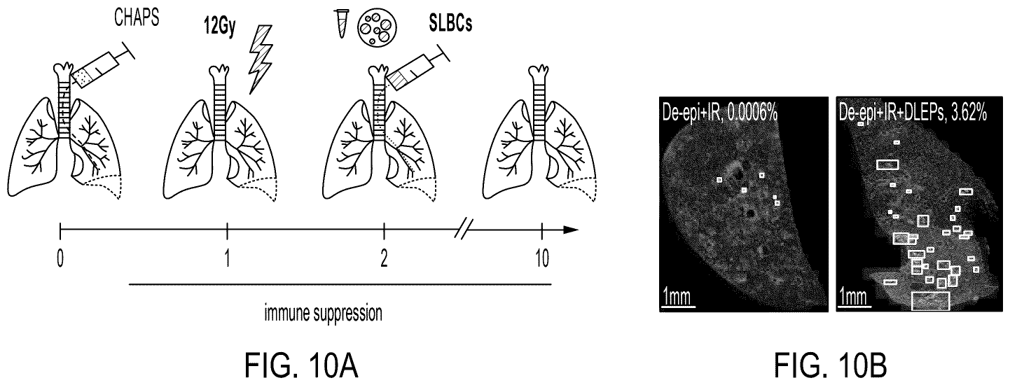

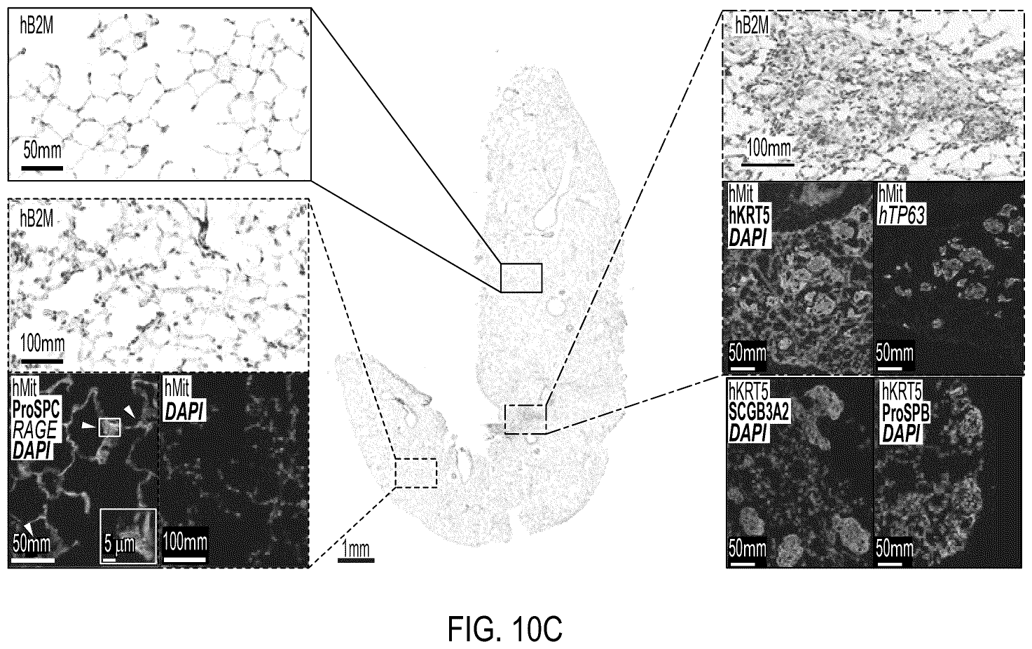

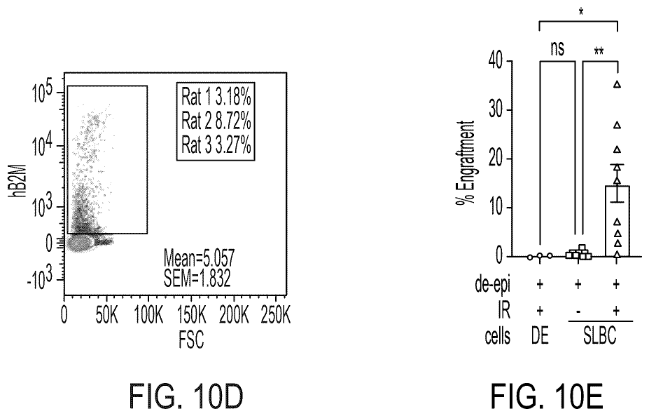

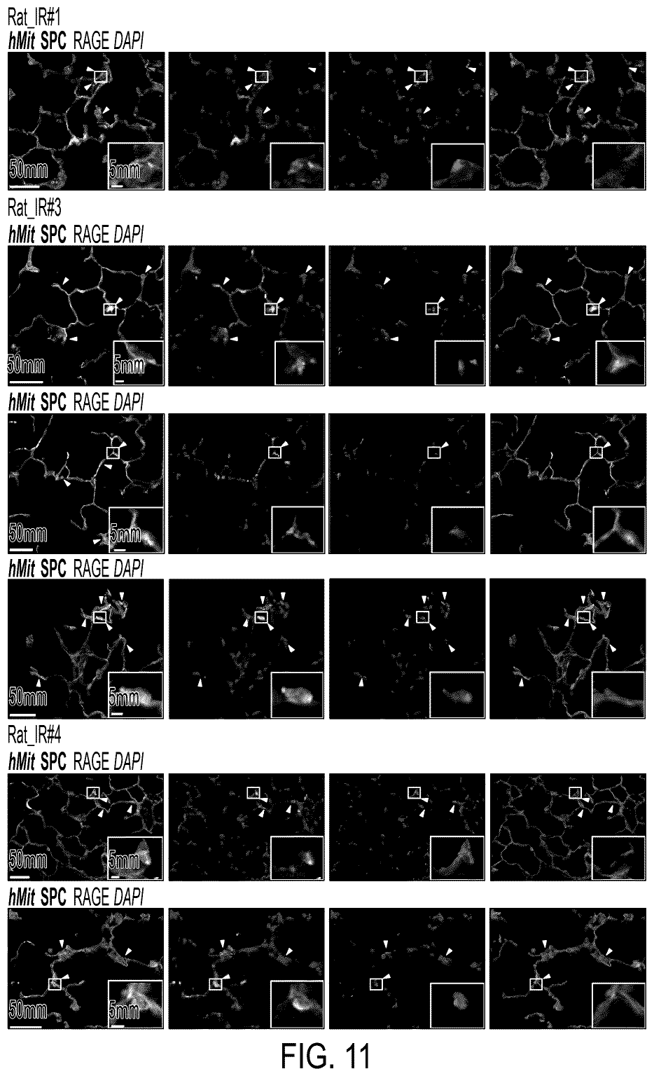

Next, we applied de-epithelialization, followed by an immune suppression [oral mycophenolate, intramuscular methylprednisolone, and subcutaneous (sq) tacrolimus], irradiation at day 1 and at day 2 administered 10⁷ DLEPs cells intrabronchially (FIG. 10A). This resulted in patchy regions (FIG. 10B) that consisted of human alveolar epithelium as evaluated by staining for human mitochondria (hMIT) and RNAscope¹⁵² for human b2-microglobulin (B2M) (FIG. 0C). In these regions, hMIT was co-expressed with RAGE and with SFTPC, indicative of AT1 and AT2 differentiation (FIG. 10C, left panels; FIG. 11). In addition, we observed dense aggregates of human cells (hMIT+ and B2M+) that stained for hKRT5 and hP63 (FIG. 10C, right panels). These structures are highly reminiscent of the KRT5-pods first identified in the distal lungs of mice and humans after severe injury.²¹ Flow cytometry for B2M of the entire lower left lung showed on average ˜5% engraftment (FIG. 10D). Genomic DNA qPCR from 150 μm thick sections for human AluYb8 compared to rat genomic DNA,¹⁵³ we estimated the number of human cells present in the section with maximal engraftment to be 0.6% of human cells/section in non-irradiated recipients (FIG. 10E), and ˜12% in irradiated recipients. Importantly, definitive endoderm cells showed no engraftment. We note that as only ˜25% of rat lung cells are epithelial,¹⁵⁴ the actual replacement fraction is in fact approximately fourfold higher.

DLEPs can convincingly engraft in rat lungs after appropriate conditioning and immunosuppression and that two patterns of engraftment were observed: KRT5-pods and integrated replacement of AT1 and AT2 cells.

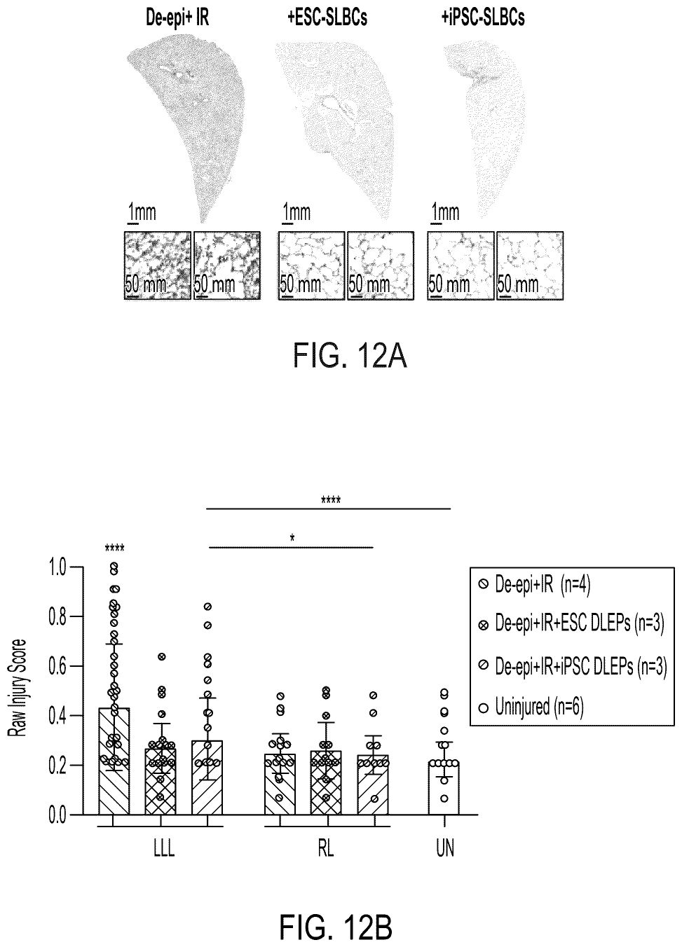

Injury repair by DLEPs. We assessed in transplanted rats the lung injury score (LIS) according to ATS guidelines.^{151,155,156} Thirty 1000×1000 μm fields were randomly selected using an ImageJ macro and assessed by a pathologist in a blinded fashion. Whereas lungs were severely injured at day 10 by the conditioning regimen, administration DLEPs resulted in a striking reduction in LIS that, importantly, quantitatively approached that of uninjured lungs (FIG. 12B). We note that injury repair appears more extensive than engraftment, suggesting paracrine effects.

### Conclusions

We have shown here that hPSC-derived cells engraft and integrate in the distal lung of rats conditioned with regional de-epithelialization followed by irradiation and promote repair of lung damage inflicted by the conditioning regimen.

Our findings that a cell population sharing expression signatures with airway BCs or secretory cells engrafted in the distal lung and promoted repair supports recent reports describing subsets of cells with BC-like and secretory-like phenotypes participating in distal lung regeneration in mice and, by inference, in humans.^{22-25,27,29,83,133}

Interestingly, DLEPs generated structures expressing KRT5 and p63.^{21,22,24} KRT5-pods with a very similar staining pattern for KRT5 and TP63 arise after severe lung injury in mice and humans.^{21,22,24,25} In another study, expansion of KRT5⁺ cells has also been demonstrated after severe lung injury in humans.¹⁵⁷ These cells co-expressed SFTPC,¹⁵⁷ consistent with the expression of SFTPC in engrafted KRT5⁺ cells in our model. DLEPs, or subpopulations thereof, therefore likely represent the equivalent to the precursors of KRT5-pods in vivo.

It is very likely that the KRT5-pods play a role in the observed repair. Conditional deletion of KRT5⁺ cells after injury in the mouse led to persistent damage after injury and reduced formation of alveolar “nets” consisting of AT1 cells.²² We observed formation of such donor-derived alveolar “nets” in our model as well. Although the quantitative contribution of KRT5⁺ cells to the alveolar epithelium is unclear, KRT5⁺ cells arising after severe lung injury may also fulfill a supportive role, either structural or mediated by paracrine factors, in lung repair.^{24,158} Consistent with this idea, the extent of injury repair by engrafted DLEPs after conditioning with de-epithelialization/irradiation was much more extensive than the actual alveolar engraftment. Our data therefore suggest that DLEPs generated from hPSCs are potentially the precursors of the human equivalent of regenerative KRT5⁺ cells in mice and provide a unique in vivo model to further investigate the biology and fate of these cells.

This pre-clinical model can be used for cell therapy for lung diseases, including those characterized by acute injury of distal lung, such as in acute respiratory distress syndrome, or by an aberrant response to chronic injury, such as in idiopathic pulmonary fibrosis.

Strategy for the Development of Cell Replacement Therapy for IPF and chILD

We developed an approach consisting of local de-epithelialization and irradiation that allows engraftment and injury repair by hPSC-derived distal lung progenitors in immunosuppressed rats. Based on these data, we will develop improved conditioning regimens that avoid irradiation.

Compared to humans, rodents have smaller alveoli,^{23,24} and do not have respiratory airways,²⁵ an airway region where gas exchange takes place but is also the niche of putative distal lung stem cells that are absent in rodents.^{19,20} The lung architecture of swine, on the other hand, is more similar to humans.^{26,27} Swine do not present the elevated cost and ethical concerns of non-human primates, and have a further advantage that, by performing initial studies on weanlings and then moving to older animals, this model can be progressively scaled to adult human size. We will therefore adapt the conditioning regimen we developed in the rat to juvenile, and then progressively older, swine to allow engraftment of hPSC-derived lung progenitors. As generating genetically modified swine models is eminently possible,^{26,28} we will, using the same principle of size scaling in the development of conditioning and engraftment approaches, generate genetically modified swine models of chILD, and in a second stage develop swine models where fibrosis is, analogous to humans with the same mutations, expected to arise later in life. We hypothesize that engraftment with human cells, possibly with attenuated conditioning because of the ongoing injury and repair response in ILD and IPF, will prevent or reverse disease progression.

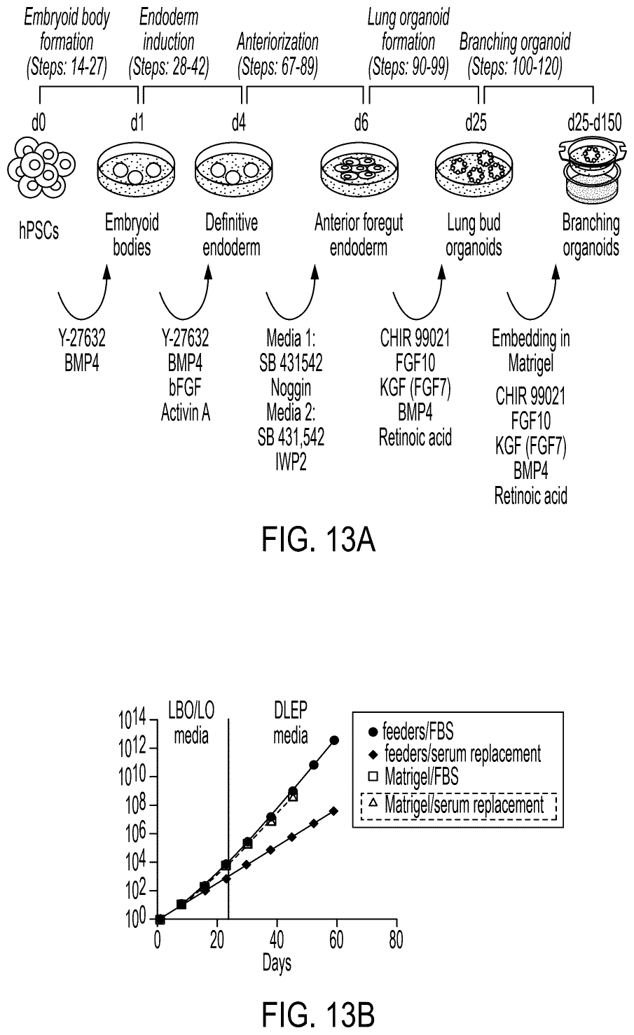

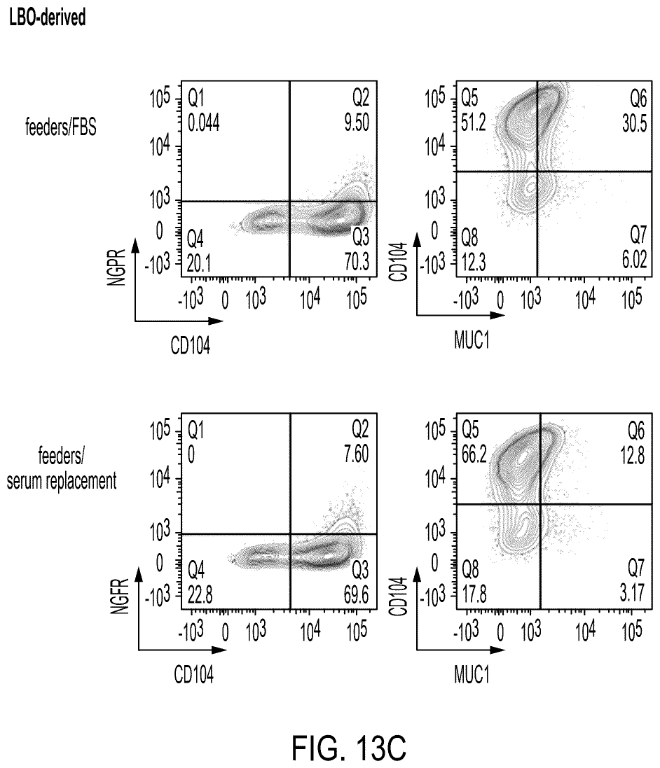

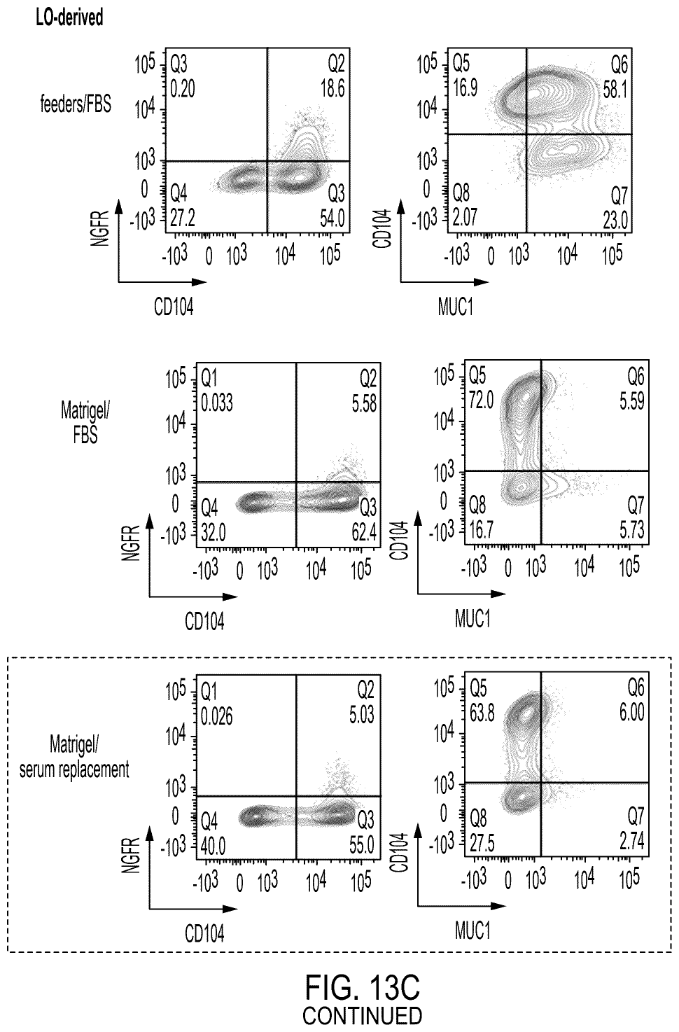

DLEPs are derived from LOs grown in Matrigel 3D cultures. The LO protocol is depicted in FIG. 13A.^{30,31,48-50} hPSCs (either ESCs or iPSCs) are first specified definitive endoderm (DE) using high concentrations of Activin A, followed by induction of anterior foregut endoderm (AFE) in defined conditions consisting of WNT, BMP and TGFb inhibition. Next, cells are resuspended as clumps and cultured in low-attachment plates where they form lung bud organoids (LBOs) in the presence of CHIR, FGF7, FGF10, dexamethasone, cyclic AMP and retinoic acid (CFKBRA). In the final stage, they are manually plated in Matrigel in the presence of the same factors, where they form branching LOs, staged at the second trimester of human gestation. DLEPs are generated from single cell suspensions of LOs and grown in the presence of 3T3-J2 cells (and fetal bovine serum (FBS)), EGF and ROCKi. DLEP generation by this method involves using Matrigel, FBS and 3T3-J2 cells, which are all animal products.

In a revised method, the Matrigel 3D step was eliminated. In these experiments, we used flow cytometry as a screening benchmark. As mentioned before, the DLEPs consist of a CD104^{hi} (BC-like) and a CD104^{lo} population (secretory-like). A fraction of the CD104^{hi}, BC-like population expresses NGFR, a BC marker (FIG. 13C). We aimed for maintenance of this profile, or for an increase in CD104^{lo} and a decrease in NGFR⁺ cells, as our data suggest that this secretory-like population contains the actual stem cells in this model (see FIG. 8F).

To accomplish this, we plated the LBO (suspension) cells for four passages in the presence of the factor cocktail used for LBOs/LOs (CFKBRA) in the presence of J2 cells followed switching to DLEP conditions with J2 feeders resulted in expansion of the cells (FIG. 13B), with maintenance of the flow cytometric profile (FIG. 13C). We subsequently replaced FBS by synthetic serum (KnockOut™ serum replacement), and feeders by Matrigel coating, which resulted in optimal cellular expansion (FIG. 13B) and maintenance of the flow cytometric profile (FIG. 13C, “Matrigel/serum replacement”). These data show that Matrigel 3D stage can be omitted, enormously simplifying the protocol in terms of manual labor and saving weeks in the protocol in terms of timeline, and that FBS and feeders can be eliminated.

We will also (1) study the scRNAseq, IF and proteomic profile, and (2) conduct transplantation into the rat model as described above.

### Conditioning of the Recipient Lung and Optimization of the Rat Model

Eliminate the Need for Local Irradiation of the Lung The rat model is useful for testing the functional capacity of the DLEPs. The model consists of bronchoscopic local de-epithelialization using a mild detergent, followed by an immune suppression [oral mycophenolate, intramuscular methylprednisolone, and subcutaneous (sq) tacrolimus], irradiation at day 1 and administration 10⁷ DLEPs intrabronchially at day 2. Engraftment (FIGS. 10A-10E, 11) and prevention of aberrant repair (FIGS. 12A-12B) were observed at day 10.

We will define the optimal conditioning strategy for DLEP engraftment by replacing irradiation by one dose of 5-fluorouracil (5FU), of CDK4/6 (G1) inhibitors (palbociclib, ribociclib and abemaciclib), or of the WEE1 inhibitor, AZD1775, that acts on the G2 checkpoint⁴² to block the endogenous regenerative response and create space for engraftment. Engraftment will be assessed by IF, scRNAseq, DNA qPCR, and determination of the lung injury score according to ATS guidelines,⁴³⁻⁴⁵ performed in a blinded fashion by a lung pathologist.

The studies will facilitate the functional testing of DLEPs expanded in conditions amenable to GMP production.

### Extension of the Observation Period

To extend the observation period, we will use custom-made slow-release pellets of tacrolimus implanted s.c. that avoid repeated s.c. injection. Tacrolimus levels and engraftment will be monitored over time. In a second approach, we will also use an immunodeficient rat model, the SRG rat (Sprague-Dawley-Rag2^{(tm2hera)}Il2rgamma^{(tm1hera)}, available from Charles River).⁴⁹ This rat model is equivalent to the NSG mouse (but does not have the NOD mutation) and lacks T, B and NK cells. In this model, the conditioning regimen likely needs to be adjusted.

Alternative immune suppression protocols, such as anti-CD2 (provided available antibodies bind rat CD2), can be explored as alternatives.

### Repair of Established Damage

DLEPs convincingly promote repair after lung damage induced by combined de-epithelialization and irradiation (FIGS. 12A-12B). As we administered cells early after injury, the administered cells in fact prevented aberrant repair and incipient fibrosis.

To study whether these cells can repair established lung damage, in a first approach we will induce distal lung damage using our established protocol as damage was severe after 10 days and only transplant at day 10. In a second approach we will administer low-dose bleomycin for 7 days, which causes more sustained fibrosis in mouse models. At day 10, 20 or 30 (fibrosis may take this long to be established), we will test three conditioning regimens: no conditioning, irradiation or 5FU only, and de-epithelialization with irradiation or 5FU. The first read-out will be 10 days after cell administration. Engraftment and repair will be assessed by IF, scRNAseq, DNA qPCR, and determination of the lung injury score according to ATS guidelines,⁴³⁻⁴⁵ performed in a blinded fashion by a lung pathologist.

It is likely that milder conditioning is sufficient, especially in the bleomycin model. This would have important implications for the future development of CRT, as local de-epithelialization may not be required when a regenerative response is ongoing. If existing damage will be repaired at least to some extent, then CRT may be an option in epithelial lung disease where interstitial damage has already been established. If not, then it is likely that any intervention in humans may need to be performed before extensive structural damage is present.

### a Preclinical Porcine Model

In contrast to rodents, where modeling IPF and chILD as well as engrafting cells in the lung has been challenging, the lung architecture in swine is more similar to that of humans.^{159,160} Swine can be genetically modified, and can be scaled to human size by weight.

### Conditioning and Engraftment in Swine Model

We will adapt the conditioning regimen (e.g. by replacing irradiation by one round cell cycle-specific agents) developed in the rat to swine under immune suppression to test engraftment of hPSC-derived lung progenitors. Initial studies will be performed in weanlings, where at least tenfold fewer cells are required and would model treatment of chILD in neonates, followed by older animals up to human body weight as scale-up of cell production progresses.

Swine Model of chILD

Although we aim to treat neonates with lethal chILD (homozygous deletion of SFTPB or ABCA3), such swine cannot be bred as the mutations are lethal.^{6,7} We will therefore generate heterozygous loss-of-function NKX2.1 mutations, which causes non-lethal respiratory distress in children in a range of severities.¹⁶¹ NKX2.1 mutation may also cause choreoathetosis and thyroid abnormalities, which, though typically mild in humans, may require additional care.

### Swine Model of IPF

We will generate swine with homozygous HPS1 deletion and heterozygous telomerase (TERT) catalytic domain mutation. HPS1 deletion causes IPF in the 3^{rd} or 4^{th} decade of life in humans (˜2-4 years in pigs), as well as albinism and mild bleeding abnormalities. TERT mutations cause IPF in the 5^{th} and 6^{th} decade of human life, but can show anticipation (i.e. disease develops earlier in life in later generations) likely because of progressive germline telomere shortening.⁷¹ We will breed three generations and assess for IPF development.

The swine models of chILD and IPF will be treated with optimized protocols and/or with drugs developed using iRAPs.

### Methods

Animals, anesthesia, and euthanasia. Sprague-Dawley (SD) rats, 8-12 weeks old, weighing 230-250 and 180-220 g were used in the experiments conducted to optimize and characterize the regional lung de-epithelialization and irradiation (n=36), and the transplant studies (n=14), respectively. Rats, both males and females, were anesthetized with isoflurane vapor (3-5%) and an intraperitoneal (IP) injection of ketamine (VEDCO, Saint Joseph, MO) (80-95 mg/kg) and xylazine (Covetrus, Portland, ME) (5-10 mg/kg). The animals were then positioned upright on a rodent workstand (Hallowell EMC, Pittsfield, MA) and endotracheally intubated with a modified cannula (JorVet, Loveland, CO). The cannula had a tightly fitting tracheal plug (cut from a 200 mL pipette tip) to seal trachea during removal of de-epithelialization solution. Throughout the whole experiment, animals, maintained on 1% isoflurane, were spontaneously breathing through the lung (right) not intubated and mechanically ventilated through the intubated lung (left). The left lung was ventilated using synchronized intermitted mandatory ventilation (SIMV) on volume control modality for single lung ventilation (Tidal volume 3 cc/Kg; PEEP 5; PIP limit 35) using the Inspira Asv Advanced Safety Animal Ventilator (Harvard Apparatus, Boston, MA). Euthanasia was performed with Isoflurane (Covetrus) (5%, 15 min) inhalation, followed by bilateral thoracotomy. Following a transverse incision of the aorta to facilitate blood release from left ventricle, the main pulmonary artery was perfused by phosphate-buffered saline (PBS, Sigma-Aldrich, St. Louis, MO) via the right ventricle with static pressure that was maintained at 13 cm H₂O above the heart until lungs, perfused, were blanched. OCT compound (Sakura Finetek, Torrance, CA) was diluted in PBS (8° C. T:2 PBS in volume) and injected into the trachea. The injection volume was equivalent to 60% of the animal's total lung capacity (TLC). A total of ten tissue samples were taken from each animal and embedded in the undiluted OCT compound for frozen sections.

In vivo de-epithelialization. The de-epithelialization solution contains 4 mM 3-[(3-cholamidopropyl)dimethylammonio]-1-propanesulfonate (CHAPS, Sigma-Aldrich), 0.5 M NaCl (Sigma-Aldrich), and 25 mM EDTA (Gibco, Waltham, MA) in deionized water. Each dose was instilled in two equal aliquots, 30 minutes apart. 30 minutes after the second instillation, the work stand was lowered to its horizontal plane position. After 15 minutes, bronchoalveolar lavage (BAL) was performed twice (1 mL of PBS each) via a 3-mL syringe. The tracheal plug was pushed down the cannula to create a temporary seal at the entrance of the trachea to facilitate the BAL. The plug was quickly retrieved after each lavage to ensure ventilation. The rats were kept on the ventilator for further 15-30 min while anesthesia was weaning off, extubated and returned to their cages.

Monitoring and blood analysis. Heart rate, oxygen saturation, and rectal temperature were monitored during each experiment using a pulse oximeter (8500, Nonin Medical, Plymouth, MN) and a digital thermometer, respectively. Tail vein blood was collected one day before and 48 hours after DE experiments and delivered for pathologic analysis. In the engraftment experiments, the plasma level of tacrolimus was measured in blood samples collected from experimental and control rats the day of cells delivery and at 7 days post-transplant. Plasma was processed with the Abbott specific kit for tacrolimus level, according to the kit manufacturer's protocol (Abbott, Abbott Park, IL) and read with the Abbott Architect machine. All samples with a tacrolimus level above 18-20 ng/ml were considered optimal for rat immunosuppression.

Lung irradiation. One day after de-epithelialization, rats were anesthetized with Isoflurane (1-3%) and placed on the animal holder of the Small Animal Radiation Research Platform (SARRP). After the total body computerized tomography (CT) scan, the lung area was contoured and selected for irradiation with a build-in software of the platform. Each rat received 12 Gy delivered into two beams, one from top and one from bottom respect to the animal.

hPSC maintenance. RUES 2 (Rockfeller University Embryonic Stem Cell Line 2, passage 20-27), Sendai Virus-induced human dermal fibroblast iPSC lines (from healthy fibroblasts, purchased from Mount Sinai Stem Cell Core Facility, passage 17-23) were cultured on mouse embryonic fibroblasts (GlobalStem, Gaithersbug, MD) plated at 17,000-20,000 cells/cm². hPSC maintenance media consisted of DMEMIF12 (Croning, Teterboro, NJ), 20% Knockout Serum Replacement (Gibco), 0.1 mM β-mercaptoethanol (Sigma-Aldrich), 1% GlutaMax (Gibco), 1% non-essential amino acids (Gibco), 0.2% primocin (InvivoGen, San Diego, CA) and 20 ng/ml FGF-2 (R&D Systems, Minneapolis, MN). Media were changed daily, and cells were passaged every 4 to 5 days using Accutase/EDTA (Innovative Cell Technologies, San Diego, CA) and plated at 10,000-12,000 cells/cm² density. Cells were maintained in an undifferentiated state in a humidified 5% CO₂ atmosphere at 37° C. The cells were tested for Mycoplasma contamination by PCR every 6 months. Karyotype was performed every 6 months.

Generation of hPSC-derived lung organoids. The hPSC-derived human lung organoids were generated as described.³³ Briefly, MEFs were depleted by passaging 5-7×10⁶ hPSCs (both ESC and iPSC) onto a Matrigel-coated 10-cm dish. Cells were maintained in hPSC media in a humidified 5% CO₂ atmosphere at 37° C. After 24 hours, cells were detached with 0.05% Trypsin/EDTA (Gibco) and distributed to the 6-well low-attachment plate containing primitive streak/embryoid body media (10 μM Y-27632, 3 ng/ml BMP4, R&D Systems) to allow embryoid body formation. Embryoid bodies (EB) were fed every day with fresh endoderm induction media (10 μM Y-27632, 0.5 ng/ml BMP4, 2.5 ng/ml FGF2 and 100 ng/ml ActivinA, R&D Systems) and maintained in a humidified 5% CO₂/5% O₂ atmosphere at 37° C. Endoderm yield was checked between 74-79 hours after exposure of EBs to Activin A from endoderm induction. Endoderm yield efficiency was determined by dissociating EBs and evaluating CXCR4 and c-KIT (BioLegend, San Diego, CA) co-expression by flow cytometry. Cells used in all experiments had >90% endoderm yield (c-Kit and CXCR4 double positive in flow analysis) and were plated on 0.2% fibronectin (R&D Systems)-coated wells at a density of 80,000 cells/cm². Cells were incubated in Anteriorization media-1 (100 ng/ml Noggin and 10 μM SB431542, R&D Systems) for 24 hours, followed by Anteriorization media-2 (10 μM SB431542 and 1 μM IWP2, R&D Systems) for another 24 hours. At the end of anterior foregut endoderm induction, cells were switched to Ventralization/Branching media (3 μM CHIR99021, 10 ng/ml FGF10, 10 ng/ml rhKGF, 10 ng/ml BMP4 and 50 nM all-trans Retinoic acid, R&D Systems) for 48 hours and three-dimensional clump formation was observed. The adherent clumps were detached by gentle pipetting and transferred to the low-attachment plate, where they folded into lung bud organoids as early as d10-d12 (LBOs).

Branching media was changed every other day until d20-d25. Optionally, LBOs were embedded in 100% Matrigel in 24-well trans-well inserts. Branching media was added after Matrigel solidified and changed every 2-3 days to facilitate proper growth into lung organoids. Culture of embedded organoids can be kept for more than 6 months.

Generation of hPSC-Derived Distal Lung Epithelial Progenitors (DLEP).

Matrigel-embedded lung organoids can be used for generating DLEP when they reach d42 development. Lung organoids were released from Matrigel by incubating with dispase (1 U/ml, Corning) for 30-45 minutes in normoxic incubator. Organoids were transferred and washed in a 15 ml-conical tube with wash media (IMDM, 5% FBS) to neutralize protease, then centrifuged at 200 g for 5 minutes. Pellet was dissociated into small cell clump to single cell with 0.05% Trypsin/EDTA in normoxic incubator for 10-12 minutes with occasional pipetting with P1000. Dissociated cells were neutralized with wash media, then centrifuged at 400 g for 4 minutes. Dissociated cells were seeded on the Mitomycin C treated 3T3-J2 feeders (20,000 cells/cm²) and cultured with DLEP media (DMEM: Ham's-F12=2:1, 6% FBS, 250 ng/ml amphotericin B (Fisher Bioreagent, Pittsburgh, PA), 25 ng/ml hydrocortisone (STEMCELL Technology, Cambridge, MA), 5 μg/ml recombinant human insulin (PeproTech, Cranbury, NJ), 8 ng/ml cholera toxin (Sigma-Aldrich), 0.1 ng/ml EGF, 5 μM Y-27632 Rock Inhibitor). DLEP media was changed every other day. DLEP cells were passaged every week and could be kept more than 20 passages with normal karyotype.

LBOs can be used for generating DLEP. LBOs were dissociated into small cell clump to single cell with 0.05% Trypsin/EDTA in normoxic incubator for 10-12 minutes with occasional pipetting with P1000. Dissociated cells were neutralized with wash media, then centrifuged at 400 g for 4 minutes. Dissociated cells were seeded on Matrigel-coated cell culture plates and cultured with the LBO media first (3 μM CHIR99021, 10 ng/ml FGF10, 10 ng/ml rhKGF, 10 ng/ml BMP4 and 50 nM all-trans Retinoic acid) for four passages, followed by switching to the DLEP media (DMEM: Ham's-F12=2:1, 6% FBS, 250 ng/ml amphotericin B (Fisher Bioreagent, Pittsburgh, PA), 25 ng/ml hydrocortisone (STEMCELL Technology, Cambridge, MA), 5 μg/ml recombinant human insulin (PeproTech, Cranbury, NJ), 8 μg/ml cholera toxin (Sigma-Aldrich), 0.1 ng/ml EGF, 5 μM Y-27632 Rock Inhibitor). DLEP media was changed every other day. DLEP cells were passaged every week and could be kept more than 20 passages with normal karyotype.

The basal cell culture media include one or more of the following: Essential 8, TeSR, DMEM F12, etc.

Serum replacement media include a serum substitute or synthetic serum, StemPro-34, StemSpan, or KnockOut™ serum replacement.

In some experiments, we also added a conditioned medium to the first cell culture medium or the second cell culture medium (e.g., containing 5 μg/ml recombinant human insulin, 8 ng/ml cholera toxin, 0.1 ng/ml EGF, 5 μM Y-27632 Rock Inhibitor). The conditioned medium was a (filtered) medium conditioned by 3T3-J2 cells, grown, e.g., in the presence of the first cell culture medium or second cell culture medium.

Cell transplantation. For the transplantation studies, rats were started on a daily triple drug immunosuppression regiment consisting of with Tacrolimus (4 mg/kg daily subcutaneous, Prograf from Astellas, Northbrook, IL), Mycophenolate (0.1 mg/500 ml water, oral, Accord, Durham, NC), and Methylprednisolone (20 mg/500 ml of water, oral, Sagent, Schaumburg, IL) 24 hours after de-epithelialization and 24 hours prior to administration of the cells. 6 hours prior to the cell delivery, rats were also treated with Methylprednisolone (5 mg/kg, intramuscular). Rats were anesthetized and intubated with the same modified cannula used for the regional de-epithelialization (JorVet) targeting the lower left lobe with of a flexible bronchoscope (1800Endoscope, Bradenton, Florida). 10⁷ cells (DLEPs resuspended in their culture media) were delivered through the cannula divided into two boluses, ventilating the animal for 15 min between them. Rats were weaned from anesthesia, returned to their cages, and euthanized as described 10 days from the de-epithelialization.

Flow Cytometry analysis. Single-cell suspensions of DLEPs were obtained dissociating with 0.05% Trypsin/EDTA in normoxic incubator for 10-12 minutes with occasional pipetting with P1000. Dissociated cells were neutralized with wash media, then centrifuged at 400 g for 4 minutes. Cells were stained with conjugated antibodies for 20 minutes in FACS buffer (1% BSA in PBS) at 4 C and then resuspended in FACS buffer with 1:1000 DAPI for live/dead cells quantification. FACS was performed on BD® LSR II Flow Cytometer (BD, Franklin Lakes, NJ) and data analyzed with FlowJo 10.

Air-liquid interphase culture. 10⁶ DLEPs were seeded onto 24-well trans-well insert and kept with DLEP media on the upper and lower chamber overnight. Cells were further cultured with PneumaCult™-Ex Plus Medium (Stem Cell Technologies) on the upper and lower chamber 48 hours, after which ALI was induced and cells maintained in PneumaCult™-ALI media (Stem Cell Technologies).

scRNAseq analysis. Single-cell gene expression profiles for ESC-derived and iPSC-derived DLEPs cells were generated with 10× Genomics Chromium Single-Cell 3′ RNAseq platform. The Columbia University Genomic core performed raw data processing with 10× Genomics Cell Ranger pipeline. The reads were mapped to a human reference genomes (GRCh38). Counts for each sample were analyzed with Seurat, separately. Cells with a unique molecular identifier (UMI) lower than 500 counts were filtered. Cells with high level of mitochondrial reads (>20% of counts) were removed. The counts were normalized, scaled and analyzed for principal component analysis (PCA) with default methods. The principal components (PC) were used to generate the uniform manifold approximation and projection (UMAP), find neighbouring cells and identify cell clusters using default Seurat parameters. During clustering the resolution was adjusted to 0.1. To estimate the level of contamination by feeder cells, known cell type specific marker genes (ZEB2, FGF7, ZEB1) were used and identified as expressed by a separate cluster of cells in both samples. The feeder-associated cluster was subsetted out and integrated analysis implemented in Seurat was repeated with identical parameters. Differentially expressed genes among clusters and sample types were identified with the FindMarkers function in Seurat, with a log fold-change threshold of 0.25 and statistical cutoff of adjusted P value (>0.5). Clusters were reordered based on top differentially gene expression similarities.

EdU (5-ethynyl-2′-deoxyuridine) incorporation. 50 mg/kg EdU (Click Chemistry Tools, Scottsdale, AZ) was administered intraperitoneally 3 hours before euthanasia to label proliferating cells. EdU incorporation was visualized using the Click-iT Plus EdU Alexa Fluor 555 Imaging kit (ThermoFisher, Fair Lawn, NJ), according to the manufacturer's instructions.

Histology and immunofluorescence (IF) staining. Frozen sections (5 μm thick) and H&E stainings from each experiment were prepared. Upon returning to the room temperature, the sections were submerged in 4% paraformaldehyde for 10 minutes, washed with PBS for 5 minutes, and permeabilized in 0.25% Triton X-100/PBS solution for 20 min. After one hour of blocking in 10% donkey serum/PBS solution, EdU staining was performed in the experiments where in vivo labeling was performed. Subsequently, primary antibodies were added to the sections diluted in 5% donkey serum/PBS solution. The slides were then incubated overnight in the dark at 4° C. The next day, following three 10-minute washes in 0.025% Triton X (ThermoFisher)-100/PBS solution, the sections were treated with secondary antibodies (in 5% donkey serum/PBS) for a 1 hr in the dark. Slides were again washed three times for 10 mins in 0.025% Triton X-100/PBS solution. DAPI staining (1:1000 in PBS) was performed for 10 mins and the slides washes for 5 mins in PBS before being mounted and sealed with nail polish. IF images were taken and processed using a Leica DMi8 system and with the Leica Stellaris for confocal imaging.

RNAscope. RNAscope stainings were performed according to the manufacturer's instructions (ACD—a Bio-Techne brand, Newark, CA) using the following probe and reagents: Beta-microglobulin (B2M)-C2 (Cat No. 1211661-C2) with RNAscope 2.5 HD Duplex Detection Ki (Chromogenic). Briefly, tissue was freshly harvested and embedded into OCT and further stored at −80° C. Tissues were sectioned in 5 μm and fixed with ice cold 4% paraformaldehyde at 4° C. for 15 min. Slides were dehydrated with ethanol and air-dried completely. A hydrophobic barrier was drawn around the tissue with ImmEdge Pen (Vector Labs, Newark, CA). Endogenous peroxidase activity was blocked with hydrogen peroxide for 10 min at room temperature (RT). Slides were treated with RNAscope Protease IV for 15-30 min at RT and then processed to run the RNAscope assay. We hybridized the probes, applied RNAscope signal amplifiers and labeled probes according to the manufacturer's instructions. The images were taken with Leica AT2 bright field whole slide scanning system with 40× magnification.

Genomic qPCR. Genomic DNA was extracted from 200-250 μm-thick cryopreserved rat lung tissue using the Zymo Quick-DNA MicroPrep Kit (Zymo, Irvine, CA), according to the manufacturer's instructions. Tissue samples were washed with water to remove residual OCT before lysing with Zymo Genomic Lysis Buffer. DNA concentration was assessed by absorbance in a spectrophotometer (Thermo Scientific NanoDrop2000c). Serially diluted human genomic DNA (0.01 pg-0 ng) was mixed with 100 ng of rat genomic DNA to determine a standard curve at each set of qPCR primers. PCR was performed on QuantStudio 5 Real-Time PCR System. qPCR was performed with denaturation at 95° C. for 10 minutes, followed by 40 cycles of amplification of 95° C. for 15 seconds, 62° C. for 5 seconds and 72° C. for 15 seconds. For quantification engraftment by human cells in rat lungs, human AluYb8 qPCR was performed in 100 ng gDNA extracted from each section of the rat lung. Human gDNA quantity was calculated from raw Cq value based on the standard curve generated from each batch. Human cell engraftment percentage was calculated on the quantity of human gDNA within 100 ng rat tissue per section.

Lung Injury Score (LIS). Hematoxylin and Eosin (H&E) staining was performed using a standard protocol. The slides were scanned using a bright field whole slide scanning system (Leica AT2 digital scanning system) with a pixel size 0.25 μm. We then developed and applied a custom script to select random and non-overlapping regions of interest from whole slide image scans of lung sections. A section from the targeted left lobe and a section from the contralateral right lobe was used for the analysis. The script was used to obtain 30 regions of interest (ROI) per sample. Each ROI was then blindly evaluated for LIS according to guidelines of the 2011 ATS Workshop Report on Features and Measurements of Experimental Acute Lung Injury in Animals¹⁵¹ by a pulmonary pathologist (AS).

Statistical analysis. All statistical tests are reported in the legends of the respective figures. All the statistical tests were performed using Prism v10 (GraphPad). A value of p<0.05 was considered statistically significant.

### REFERENCES

1. Lederer, D. J. & Martinez, F. J. Idiopathic Pulmonary Fibrosis. N Engl J Med 378, 1811-1823 (2018).
2. Renzoni, E. A., Poletti, V. & Mackintosh, J. A. Disease pathology in fibrotic interstitial lung disease: is it all about usual interstitial pneumonia? The Lancet 398, 1437-1449 (2021).
3. Zhang, D. et al. Rare and Common Variants in KIF15 Contribute to Genetic Risk of Idiopathic Pulmonary Fibrosis. Am J Respir Crit Care Med 206, 56-69 (2022).
4. Alder, J. K. & Armanios, M. Telomere-mediated lung disease. Physiol Rev 102, 1703-1720 (2022).
5. Katzen, J. & Beers, M. F. Contributions of alveolar epithelial cell quality control to pulmonary fibrosis. J Clin Invest (2020) doi:10.1172/jci139519.
6. Singh, J., Jaffe, A., Schultz, A. & Selvadurai, H. Surfactant protein disorders in childhood interstitial lung disease. Eur J Pediatr 180, 2711-2721 (2021).
7. Nogee, L. M. Genetic causes of surfactant protein abnormalities. Curr. Opin. Pediatr. 31, 330-339 (2019).
8. Noble, P. W. et al. Pirfenidone in patients with idiopathic pulmonary fibrosis (CAPACITY): two randomised trials. Lancet 377, 1760-1769 (2011).
9. King, T. E., Jr. et al. A phase 3 trial of pirfenidone in patients with idiopathic pulmonary fibrosis. The New England journal of medicine 370, 2083-92 (2014).
10. Richeldi, L. et al. Efficacy and safety of nintedanib in idiopathic pulmonary fibrosis. The New England journal of medicine 370, 2071-82 (2014).
11. Bos, S., Vos, R., Van Raemdonck, D. E. & Verleden, G. M. Survival in adult lung transplantation: where are we in 2020? Curr Opin Organ Transplant 25, 268-273 (2020).
12. Florissi, I. et al. Racial Disparities in Waiting List Outcomes of Patients Listed for Lung Transplantation. Ann Thorac Surg S0003-4975(23)00918-9 (2023)
13. Malas, J. et al. Lung transplantation outcomes in patients from socioeconomically distressed communities. J Heart Lung Transplant 42, 1690-1699 (2023).
14. Riley, L. E. & Lascano, J. Gender and racial disparities in lung transplantation in the United States. J Heart Lung Transplant 40, 963-969 (2021).
15. Goobie, G. C. et al. Neighborhood-Level Disadvantage Impacts on Patients with Fibrotic Interstitial Lung Disease. Am J Respir Crit Care Med 205, 459-467 (2022).
16. Swaminathan A. C. et al. Disparities in Lung Transplant among Patients with Idiopathic Pulmonary Fibrosis: An Analysis of the IPF-PRO Registry. Ann Am Thorac Soc 19, 981-990 (2022).
17. Mooney, J. J. et al., Racial and ethnic disparities in lung transplant listing and waitlist outcomes. J Heart Lung Transplant 37, 394-400 (2018).
18. Barkauskas, C. E. et al. Type 2 alveolar cells are stem cells in adult lung. J Clin Invest 123, 3025-3036 (2013).
19. Zacharias, W. J. et al. Regeneration of the lung alveolus by an evolutionarily conserved epithelial progenitor. Nature 555, 251-255 (2018).
20. Nabhan, A. N. et al., Single-cell Wnt signaling niches maintain stemness of alveolar type 2 cells. Science 359, 1118-1123 (2018).
21. Kumar, P. A. et al. Distal airway stem cells yield alveoli in vitro and during lung regeneration following H1N1 influenza infection. Cell 147, 525-538 (2011).
22. Zuo, W. et al. p63(+)Krt5(+) distal airway stem cells are essential for lung regeneration. Nature 517, 616-620 (2015).
23. Strunz, M. et al. Alveolar regeneration through a Krt8+ transitional stem cell state that persists in human lung fibrosis. Nat Commun 11, 3559 (2020).
24. Vaughan, A. E. et al. Lineage-negative progenitors mobilize to regenerate lung epithelium after major injury. Nature 517, 621-625 (2015).
25. Kathiriya, J. J. et al., Distinct Airway Epithelial Stem Cells Hide among Club Cells but Mobilize to Promote Alveolar Regeneration. Cell Stem Cell 26, 346-358.e4 (2020).
26. Kadur Lakshminarasimha Murthy, P. et al. Human distal lung maps and lineage hierarchies reveal a bipotent progenitor. Nature 604, 111-119 (2022).
27. Basil, M. C. et al. Human distal airways contain a multipotent secretory cell that can regenerate alveoli. Nature 604, 120-126 (2022).
28. Choi, J. et al. Inflammatory Signals Induce AT2 Cell-Derived Damage-Associated Transient Progenitors that Mediate Alveolar Regeneration. Cell Stem Cell 27, 366-382.e7 (2020).
29. Choi, J. et al. Release of Notch activity coordinated by IL-1β signalling confers differentiation plasticity of airway progenitors via Fos12 during alveolar regeneration. Nat Cell Biol 23, 953-966 (2021).
30. Habermann, A. C. et al. Single-cell RNA sequencing reveals profibrotic roles of distinct epithelial and mesenchymal lineages in pulmonary fibrosis. Sci Adv 6, eaba1972 (2020).
31. Adams, T. S. et al. Single-cell RNA-seq reveals ectopic and aberrant lung-resident cell populations in idiopathic pulmonary fibrosis. Sci Adv 6, eaba1983 (2020).
32. Yamanaka, S. Pluripotent Stem Cell-Based Cell Therapy-Promise and Challenges. Cell Stem Cell 27, 523-531 (2020).
33. Matkovic Leko, I. et al. A distal lung organoid model to study interstitial lung disease, viral infection and human lung development. Nat Protoc 18, 2283-2312 (2023).
34. Chen, Y.-W. et al. A three-dimensional model of human lung development and disease from pluripotent stem cells. Nat Cell Biol 19, 542-549 (2017).
35. Huang, S. X. L. et al. Efficient generation of lung and airway epithelial cells from human pluripotent stem cells. Nat Biotechnol 32, 84-91 (2014).
36. Huang, S. X. L. et al. The in vitro generation of lung and airway progenitor cells from human pluripotent stem cells. Nat Protoc 10, 413-425 (2015).
37. Green, M. D. et al. Generation of anterior foregut endoderm from human embryonic and induced pluripotent stem cells. Nat Biotechnol 29, 267-272 (2011).
38. Strikoudis, A. et al. Modeling of Fibrotic Lung Disease Using 3D Organoids Derived from Human Pluripotent Stem Cells. Cell Rep 27, 3709-3723.e5 (2019).
39. Pezet, M. G. et al. Human pluripotent stem cell-derived respiratory airway progenitors generate alveolar epithelial cells and recapitulate features of idiopathic pulmonary fibrosis. bioRxiv 2023.01.30.526265 (2023) doi:10.1101/2023.01.30.526265.
40. Guha, A., Deshpande, A., Jain, A., Sebastiani, P. & Cardoso, W. V. Uroplakin 3a+ Cells Are a Distinctive Population of Epithelial Progenitors that Contribute to Airway Maintenance and Post-injury Repair. Cell Rep 19, 246-254 (2017).
41. Liu, H.-Y. et al. Engraftment and injury repair in regionally conditioned rat lung in vivo by lung progenitors derived from human pluripotent stem cells. bioRxiv 2023.11.28.569060 (2023) doi:10.1101/2023.11.28.569060.
42. Wijsenbeek, M. & Cottin, V. Spectrum of Fibrotic Lung Diseases. New England Journal of Medicine 383, 958-968 (2020).
43. Wang, Y. et al. Genetic defects in surfactant protein A2 are associated with pulmonary fibrosis and lung cancer. Am J Hum Genet 84, 52-59 (2009).
44. van Moorsel, C. H. M. et al. SFTPA2 Mutations in Familial and Sporadic Idiopathic Interstitial Pneumonia. Am J Respir Crit Care Med 192, 1249-1252 (2015).
45. Nathan, N. et al. Germline SFTPA1 mutation in familial idiopathic interstitial pneumonia and lung cancer. Hum Mol Genet 25, 1457-1467 (2016).
46. Maitra, M., Wang, Y., Gerard, R. D., Mendelson, C. R. & Garcia, C. K. Surfactant protein A2 mutations associated with pulmonary fibrosis lead to protein instability and endoplasmic reticulum stress. J Biol Chem 285, 22103-22113 (2010).
47. Nogee, L. M. et al. A mutation in the surfactant protein C gene associated with familial interstitial lung disease. N Engl J Med 344, 573-579 (2001).
48. van Moorsel, C. H. M. et al. Surfactant protein C mutations are the basis of a significant portion of adult familial pulmonary fibrosis in a dutch cohort. Am J Respir Crit Care Med 182, 1419-1425 (2010).
49. Lawson, W. E. et al. Endoplasmic reticulum stress enhances fibrotic remodeling in the lungs. Proceedings of the National Academy of Sciences of the United States of America 108, 10562-7 (2011).
50. Beers, M. F. Inhibition of cellular processing of surfactant protein C by drugs affecting intracellular pH gradients. The Journal of biological chemistry 271, 14361-70 (1996).
51. Zhong, Q. et al. Role of endoplasmic reticulum stress in epithelial-mesenchymal transition of alveolar epithelial cells: effects of misfolded surfactant protein. American journal of respiratory cell and molecular biology 45, 498-509 (2011).
52. Maguire, J. A., Mulugeta, S. & Beers, M. F. Endoplasmic reticulum stress induced by surfactant protein C BRICHOS mutants promotes proinflammatory signaling by epithelial cells. Am J Respir Cell Mol Biol 44, 404-414 (2011).
53. Seward, S. L., Jr. & Gahl, W. A. Hermansky-Pudlak syndrome: health care throughout life. Pediatrics 132, 153-60 (2013).
54. Vicary, G. W., Vergne, Y., Santiago-Cornier, A., Young, L. R. & Roman, J. Pulmonary Fibrosis in Hermansky-Pudlak Syndrome. Ann Am Thorac Soc (2016) doi:10.1513/AnnalsATS.201603-186FR.
55. Anderson, P. D. et al., Hermansky-Pudlak syndrome type 4 (HPS-4): clinical and molecular characteristics. Hum Genet 113, 10-7 (2003).
56. Hengst, M. et al. Hermansky-Pudlak syndrome type 2 manifests with fibrosing lung disease early in childhood. Orphanet J Rare Dis 13, 42 (2018).
57. Luzio, J. P., Hackmann, Y., Dieckmann, N. M. & Griffiths, G. M. The biogenesis of lysosomes and lysosome-related organelles. Cold Spring Harb Perspect Biol 6, a016840 (2014).
58. Huizing, M. et al., Disorders of lysosome-related organelle biogenesis: clinical and molecular genetics. Annu Rev Genomics Hum Genet 9, 359-86 (2008).
59. Li, W. et al. Murine Hermansky-Pudlak syndrome genes: regulators of lysosome-related organelles. Bioessays 26, 616-28 (2004).
60. Tomer, Y. et al. The common ABCA3E292V variant disrupts AT2 cell quality control and increases susceptibility to lung injury and aberrant remodeling. Am J Physiol Lung Cell Mol Physiol 321, L291-L307 (2021).
61. Shulenin, S. et al. ABCA3 gene mutations in newborns with fatal surfactant deficiency. The New England journal of medicine 350, 1296-303 (2004).
62. Peca, D. et al., ABCA3, a key player in neonatal respiratory transition and genetic disorders of the surfactant system. Biochem Soc Trans 43, 913-919 (2015).
63. Klay, D. et al., ABCA3 mutations in adult pulmonary fibrosis patients: a case series and review of literature. Curr Opin Pulm Med 26, 293-301 (2020).
64. Wambach, J. A. et al. Genotype-phenotype correlations for infants and children with ABCA3 deficiency. Am J Respir Crit Care Med 189, 1538-1543 (2014).
65. Diaz de Leon, A. et al. Subclinical lung disease, macrocytosis, and premature graying in kindreds with telomerase (TERT) mutations. Chest 140, 753-763 (2011).
66. Stuart, B. D. et al. Exome sequencing links mutations in PARN and RTEL1 with familial pulmonary fibrosis and telomere shortening. Nat Genet 47, 512-517 (2015).
67. Armanios, M. Syndromes of telomere shortening. Annu Rev Genomics Hum Genet 10, 45-61 (2009).
68. Armanios, M. Telomerase mutations and the pulmonary fibrosis-bone marrow failure syndrome complex. The New England journal of medicine 367, 384; author reply 384 (2012).
69. Armanios, M. Y. et al. Telomerase mutations in families with idiopathic pulmonary fibrosis. The New England journal of medicine 356, 1317-26 (2007).
70. Garcia, C. K. Running short on time: lung transplant evaluation for telomere-related pulmonary fibrosis. Chest 147, 1450-2 (2015).
71. Alder, J. K. & Armanios, M. Telomere-mediated Lung Disease. Physiol Rev (2022) doi:10.1152/physrev.00046.2021.
72. Kelich, J. et al. Telomere dysfunction implicates POT1 in patients with idiopathic pulmonary fibrosis. J Exp Med 219, e20211681 (2022).
73. Dodson, L. M. et al. From incomplete penetrance with normal telomere length to severe disease and telomere shortening in a family with monoallelic and biallelic PARN pathogenic variants. Hum Mutat 40, 2414-2429 (2019).
74. Cogan, J. D. et al. Rare variants in RTEL1 are associated with familial interstitial pneumonia. Am J Respir Crit Care Med 191, 646-655 (2015).
75. Stanley, S. E. et al. Loss-of-function mutations in the RNA biogenesis factor NAF1 predispose to pulmonary fibrosis-emphysema. Sci Transl Med 8, 351ra107 (2016).
76. Zhang, D. et al. Homozygous Rare PARN Missense Mutation in Familial Pulmonary Fibrosis. Am J Respir Crit Care Med 199, 797-799 (2019).
77. Petrovski, S. et al. An Exome Sequencing Study to Assess the Role of Rare Genetic Variation in Pulmonary Fibrosis. Am J Respir Crit Care Med 196, 82-93 (2017).
78. Gonzilez-Quiroz, M. et al. When Endoplasmic Reticulum Proteostasis Meets the DNA Damage Response. Trends Cell Biol 30, 881-891 (2020).
79. Haneke, K. et al. CDK1 couples proliferation with protein synthesis. J Cell Biol 219, e201906147 (2020).
80. Matos, L., Gouveia, A. M. & Almeida, H. ER Stress Response in Human Cellular Models of Senescence. J Gerontol A Biol Sci Med Sci 70, 924-935 (2015).
81. Borok, Z. et al. Grp78 Loss in Epithelial Progenitors Reveals an Age-linked Role for Endoplasmic Reticulum Stress in Pulmonary Fibrosis. Am J Respir Crit Care Med 201, 198-211 (2020).
82. Chilosi, M. et al. Abnormal Re-epithelialization and Lung Remodeling in Idiopathic Pulmonary Fibrosis: The Role of ΔN-p63. Laboratory Investigation 82, 1335-1345 (2002).
83. Kobayashi, Y. et al. Persistence of a regeneration-associated, transitional alveolar epithelial cell state in pulmonary fibrosis. Nat Cell Biol 22, 934-946 (2020).
84. Wu, H. et al. Progressive Pulmonary Fibrosis Is Caused by Elevated Mechanical Tension on Alveolar Stem Cells. Cell 180, 107-121.e17 (2020).
85. Bridges, J. P., Wert, S. E., Nogee, L. M. & Weaver, T. E. Expression of a human surfactant protein C mutation associated with interstitial lung disease disrupts lung development in transgenic mice. J Biol Chem 278, 52739-52746 (2003).
86. Wang, W.-J. et al., Deletion of exon 4 from human surfactant protein C results in aggresome formation and generation of a dominant negative. J Cell Sci 116, 683-692 (2003).
87. Mulugeta, S. et al., A surfactant protein C precursor protein BRICHOS domain mutation causes endoplasmic reticulum stress, proteasome dysfunction, and caspase 3 activation. Am J Respir Cell Mol Biol 32, 521-530 (2005).
88. Conkright, J. J. et al., Overexpression of surfactant protein-C mature peptide causes neonatal lethality in transgenic mice. Am J Respir Cell Mol Biol 26, 85-90 (2002).
89. Nureki, S.-I. et al. Expression of mutant Sftpc in murine alveolar epithelia drives spontaneous lung fibrosis. J Clin Invest 128, 4008-4024 (2018).
90. Tashiro, J. et al. Exploring Animal Models That Resemble Idiopathic Pulmonary Fibrosis. Front Med (Lausanne) 4, 118 (2017).
91. Mathai, S. K., Newton, C. A., Schwartz, D. A. & Garcia, C. K. Pulmonary fibrosis in the era of stratified medicine. Thorax 71, 1154-1160 (2016).
92. Sutton, R. M. et al. Rare surfactant-related variants in familial and sporadic pulmonary fibrosis. Hum Mutat 43, 2091-2101 (2022).
93. Basil, M. C. et al. Human distal airways contain a multipotent secretory cell that can regenerate alveoli. Nature 604, 120-126 (2022).
94. Jacob, A. et al. Derivation of self-renewing lung alveolar epithelial type II cells from human pluripotent stem cells. Nat Protoc 14, 3303-3332 (2019).
95. Jacob, A. et al. Differentiation of Human Pluripotent Stem Cells into Functional Lung Alveolar Epithelial Cells. Cell Stem Cell 21, 472-488.e10 (2017).
96. Yamamoto, Y. et al. Long-term expansion of alveolar stem cells derived from human iPS cells in organoids. Nat Methods 14, 1097-1106 (2017).
97. Olmeda, B., Martinez-Calle, M. & Perez-Gil, J. Pulmonary surfactant metabolism in the alveolar airspace: Biogenesis, extracellular conversions, recycling. Ann Anat 209, 78-92 (2017).
98. Brehier, A. & Rooney, S. A. Phosphatidylcholine synthesis and glycogen depletion in fetal mouse lung: developmental changes and the effects of dexamethasone. Exp Lung Res 2, 273-287 (1981).
99. Chi, E. Y. The ultrastructural study of glycogen and lamellar bodies in the development of fetal monkey lung. Exp Lung Res 8, 275-289 (1985).
100. Kadur Lakshminarasimha Murthy, P. et al. Human distal lung maps and lineage hierarchies reveal a bipotent progenitor. Nature 604, 111-119 (2022).
101. Dobbs, L. G., Gonzalez, R. F., Allen, L. & Froh, D. K. HTI56, an integral membrane protein specific to human alveolar type I cells. J Histochem Cytochem 47, 129-137 (1999).
102. Sikkema, L. et al. An integrated cell atlas of the lung in health and disease. Nat Med 29, 1563-1577 (2023).
103. Liu, Z. et al. MAPK-Mediated YAP Activation Controls Mechanical-Tension-Induced Pulmonary Alveolar Regeneration. Cell Rep 16, 1810-1819 (2016).
104. Wu, H. et al. Progressive Pulmonary Fibrosis Is Caused by Elevated Mechanical Tension on Alveolar Stem Cells. Cell 180, 107-121.e17 (2020).
105. Shiraishi, K. et al. Biophysical forces mediated by respiration maintain lung alveolar epithelial cell fate. Cell 186, 1478-1492.e15 (2023).
106. Chung, M.-I., Bujnis, M., Barkauskas, C. E., Kobayashi, Y. & Hogan, B. L. M. Niche-mediated BMP/SMAD signaling regulates lung alveolar stem cell proliferation and differentiation. Development 145, dev163014 (2018).
107. Gokey, J. J., Patel, S. D. & Kropski, J. A. The Role of Hippo/YAP Signaling in Alveolar Repair and Pulmonary Fibrosis. Front Med (Lausanne) 8, 752316 (2021).
108. Kastan, N. et al. Small-molecule inhibition of Lats kinases may promote Yap-dependent proliferation in postmitotic mammalian tissues. Nat Commun 12, 3100 (2021).
109. Nantie, L. B. et al. Lats1/2 inactivation reveals Hippo function in alveolar type I cell differentiation during lung transition to air breathing. Development 145, dev163105 (2018).
110. van Soldt, B. J. et al., Hippo-Yap/Taz signaling: Complex network interactions and impact in epithelial cell behavior. Wiley Interdiscip Rev Dev Biol 9, e371 (2020).
111. Banushi, B. & Simpson, F. Overlapping Machinery in Lysosome-Related Organelle Trafficking: A Lesson from Rare Multisystem Disorders. Cells 11, 3702 (2022).
112. Delevoye, C., Marks, M. S. & Raposo, G. Lysosome-related organelles as functional adaptations of the endolysosomal system. Curr Opin Cell Biol 59, 147-158 (2019).
113. Vicary, G. W., Vergne, Y., Santiago-Cornier, A., Young, L. R. & Roman, J. Pulmonary Fibrosis in Hermansky-Pudlak Syndrome. Ann Am Thorac Soc 13, 1839-1846 (2016).
114. Introne, W. J., Huizing, M., Malicdan, M. C. V., O'Brien, K. J. & Gahl, W. A. Hermansky-Pudlak Syndrome. in GeneReviews® (eds. Adam, M. P. et al.) (University of Washington, Seattle, Seattle (WA), 1993).
115. Li, W. et al. New insights into the pathogenesis of Hermansky-Pudlak syndrome. Pigment Cell Melanoma Res 35, 290-302 (2022).
116. Katzen, J. & Beers, M. F. Contributions of alveolar epithelial cell quality control to pulmonary fibrosis. J Clin Invest 130, 5088-5099 (2020).
117. Kobayashi, Y. et al. Persistence of a regeneration-associated, transitional alveolar epithelial cell state in pulmonary fibrosis. Nat Cell Biol 22, 934-946 (2020).
118. Perkins, T. N. & Oury, T. D. The perplexing role of RAGE in pulmonary fibrosis: causality or casualty? Ther Adv Respir Dis 15, 17534666211016071 (2021).
119. Konkimalla, A. et al. Transitional cell states sculpt tissue topology during lung regeneration. Cell Stem Cell 30, 1486-1502.e9 (2023).
120. Habermann, A. C. et al. Single-cell RNA sequencing reveals profibrotic roles of distinct epithelial and mesenchymal lineages in pulmonary fibrosis. Sci Adv 6, eaba1972 (2020).
121. Basil, M. C. et al. The Cellular and Physiological Basis for Lung Repair and Regeneration: Past, Present, and Future. Cell Stem Cell 26, 482-502 (2020).
122. Barkauskas, C. E. et al. Type 2 alveolar cells are stem cells in adult lung. J Clin Invest 123, 3025-3036 (2013).
123. Zacharias, W. J. et al. Regeneration of the lung alveolus by an evolutionarily conserved epithelial progenitor. Nature 555, 251-255 (2018).
124. Nabhan, A. N. et al., Single-cell Wnt signaling niches maintain stemness of alveolar type 2 cells. Science 359, 1118-1123 (2018).
125. Strunz, M. et al. Alveolar regeneration through a Krt8+ transitional stem cell state that persists in human lung fibrosis. Nat Commun 11, 3559 (2020).
126. Penkala, I. J. et al. Age-dependent alveolar epithelial plasticity orchestrates lung homeostasis and regeneration. Cell Stem Cell 28, 1775-1789.e5 (2021).
127. Kim, C. F. B. et al. Identification of bronchioalveolar stem cells in normal lung and lung cancer. Cell 121, 823-835 (2005).
128. Liu, Q. et al. Lung regeneration by multipotent stem cells residing at the bronchioalveolar-duct junction. Nat Genet 51, 728-738 (2019).
129. Taylor, M. S. et al. A Conserved Distal Lung Regenerative Pathway in Acute Lung Injury. Am J Pathol 188, 1149-1160 (2018).
130. Guha, A. et al. Analysis of Notch signaling-dependent gene expression in developing airways reveals diversity of Clara cells. PLoS One 9, e88848 (2014).
131. Guha, A., Deshpande, A., Jain, A., Sebastiani, P. & Cardoso, W. V. Uroplakin 3a+ Cells Are a Distinctive Population of Epithelial Progenitors that Contribute to Airway Maintenance and Post-injury Repair. Cell Rep 19, 246-254 (2017).
132. Melms, J. C. et al. A molecular single-cell lung atlas of lethal COVID-19. Nature 595, 114-119 (2021).
133. Kadur Lakshminarasimha Murthy, P. et al. Human distal lung maps and lineage hierarchies reveal a bipotent progenitor. Nature 604, 111-119 (2022).
134. Habermann, A. C. et al. Single-cell RNA sequencing reveals profibrotic roles of distinct epithelial and mesenchymal lineages in pulmonary fibrosis. Sci Adv 6, eaba1972 (2020).
135. Weiner, A. I. et al. Mesenchyme-free expansion and transplantation of adult alveolar progenitor cells: steps toward cell-based regenerative therapies. NPJ Regen Med 4, 17 (2019).
136. Louie, S. M. et al. Progenitor potential of lung epithelial organoid cells in a transplantation model. Cell Rep 39, 110662 (2022).
137. Álvarez-Palomo, B. et al. Evaluation of the Spanish population coverage of a prospective HLA haplobank of induced pluripotent stem cells. Stem Cell Res Ther 12, 233 (2021).
138. Byrne, A. T. et al. Interrogating open issues in cancer precision medicine with patient-derived xenografts. Nat Rev Cancer 17, 254-268 (2017).
139. Ma, L. et al. Airway stem cell reconstitution by the transplantation of primary or pluripotent stem cell-derived basal cells. Cell Stem Cell 30, 1199-1216.e7 (2023).
140. Herriges, M. J. et al. Durable alveolar engraftment of PSC-derived lung epithelial cells into immunocompetent mice. Cell Stem Cell 30, 1217-1234.e7 (2023).
141. Nikolić, M. Z. et al. Human embryonic lung epithelial tips are multipotent progenitors that can be expanded in vitro as long-term self-renewing organoids. Elife 6, e26575 (2017).
142. Miller, A. J. et al. In Vitro Induction and In Vivo Engraftment of Lung Bud Tip Progenitor Cells Derived from Human Pluripotent Stem Cells. Stem Cell Reports 10, 101-119 (2018).
143. Dye, B. R. et al. A bioengineered niche promotes in vivo engraftment and maturation of pluripotent stem cell derived human lung organoids. Elife 5, e19732 (2016).
144. Chen, Y.-W. et al. A three-dimensional model of human lung development and disease from pluripotent stem cells. Nat Cell Biol 19, 542-549 (2017).
145. Strikoudis, A. et al. Modeling of Fibrotic Lung Disease Using 3D Organoids Derived from Human Pluripotent Stem Cells. Cell Rep 27, 3709-3723.e5 (2019).
146. Matkovic Leko et al. A distal lung organoid model to study interstitial lung disease, viral infection and human lung development. Nat Protoc (2023) doi:10.1038/s41596-023-00827-6.
147. Butler, C. R. et al. Rapid Expansion of Human Epithelial Stem Cells Suitable for Airway Tissue Engineering. Am J Respir Crit Care Med 194, 156-168 (2016).
148. Montoro, D. T. et al. A revised airway epithelial hierarchy includes CFTR-expressing ionocytes. Nature 560, 319-324 (2018).
149. Guha, A. et al. Neuroepithelial body microenvironment is a niche for a distinct subset of Clara-like precursors in the developing airways. Proc Natl Acad Sci USA 109, 12592-12597 (2012).
150. Tsao, P.-N. et al. Notch signaling controls the balance of ciliated and secretory cell fates in developing airways. Development 136, 2297-2307 (2009).
151. Kulkarni, H. S. et al. Update on the Features and Measurements of Experimental Acute Lung Injury in Animals: An Official American Thoracic Society Workshop Report. Am J Respir Cell Mol Biol 66, e1-e14 (2022).
152. Wang, F. et al. RNAscope: a novel in situ RNA analysis platform for formalin-fixed, paraffin-embedded tissues. J Mol Diagn 14, 22-29 (2012).
153. Prigent, J. et al. Human Progenitor Cell Quantification after Xenotransplantation in Rat and Mouse Models by a Sensitive qPCR Assay. Cell Transplant 24, 1639-1652 (2015).
154. Haies, D. M. et al., Morphometric study of rat lung cells. I. Numerical and dimensional characteristics of parenchymal cell population. Am Rev Respir Dis 123, 533-541 (1981).
155. Dorrello, N. V. et al. Functional vascularized lung grafts for lung bioengineering. Sci Adv 3, e1700521 (2017).
156. Toth, A. et al. Inflammatory blockade prevents injury to the developing pulmonary gas exchange surface in preterm primates. Sci Transl Med 14, eab18574 (2022).
157. Kathiriya, J. J. et al. Human alveolar type 2 epithelium transdifferentiates into metaplastic KRTS+ basal cells. Nat Cell Biol 24, 10-23 (2022).
158. Basil, M. C. et al. The Cellular and Physiological Basis for Lung Repair and Regeneration: Past, Present, and Future. Cell Stem Cell 26, 482-502 (2020).
159. Lunney, J. K. et al. Importance of the pig as a human biomedical model. Sci Transl Med 13, eabd5758 (2021).
160. Judge, E. P. et al. Anatomy and bronchoscopy of the porcine lung. A model for translational respiratory medicine. Am J Respir Cell Mol Biol 51, 334-343 (2014).
161. Krude, H. et al. Choreoathetosis, hypothyroidism, and pulmonary alterations due to human NKX2-1 haploinsufficiency. J Clin Invest 109, 475-480 (2002).
162. Chen, G. et al. Chemically defined conditions for human iPSC derivation and culture. Nat Methods 8, 424-429 (2011).
163. Sun, N. et al. Feeder-free derivation of induced pluripotent stem cells from adult human adipose stem cells. Proc Natl Acad Sci USA 106, 15720-15725 (2009).

The scope of the present invention is not limited by what has been specifically shown and described hereinabove. Those skilled in the art will recognize that there are suitable alternatives to the depicted examples of materials, configurations, constructions and dimensions. Numerous references, including patents and various publications, are cited and discussed in the description of this invention. The citation and discussion of such references is provided merely to clarify the description of the present invention and is not an admission that any reference is prior art to the invention described herein. All references cited and discussed in this specification are incorporated herein by reference in their entirety. Variations, modifications and other implementations of what is described herein will occur to those of ordinary skill in the art without departing from the spirit and scope of the invention. While certain embodiments of the present invention have been shown and described, it will be obvious to those skilled in the art that changes and modifications may be made without departing from the spirit and scope of the invention. The matter set forth in the foregoing description is offered by way of illustration only and not as a limitation.

What is claimed is:

1. A method for generating lung progenitor cells, the method comprising:
(a) producing anterior foregut endoderm cells from mammalian pluripotent stem cells (PSCs);
(b) culturing the anterior foregut endoderm cells in a suspension culture comprising a glycogen synthase kinase (GSK) inhibitor, a bone morphogenic protein (BMP) agonist, at least one FGF agonists, and retinoic acid, to generate a lung bud organoid (LBO);
(c) dissociating the LBO and culturing the dissociated LBO on a first cell culture substrate in presence of a GSK inhibitor, a BMP agonist, at least one FGF agonist, and retinoic acid, to generate plated LBO, wherein the first cell culture substrate is coated with a first biomolecule; and
(d) culturing the plated LBO on a second cell culture substrate in a first culture medium or a second culture medium, wherein the first culture medium comprises an inhibitor of Rho kinase (ROCK), a GSK inhibitor, at least one FGF agonist, a BMP agonist, and retinoic acid, wherein the second culture medium comprises insulin, an EGF agonist, a corticosteroid, a 3′,5′-cyclic adenosine monophosphate (cAMP) pathway activator, and an inhibitor of ROCK, wherein the second cell culture substrate is coated with a second biomolecule.

2. The method of claim 1, wherein the GSK inhibitor is CHIR99021.

3. The method of claim 1, wherein the GSK inhibitor is at a concentration ranging from about 1 μM to about 10 μM.

4. The method of claim 1, wherein the GSK inhibitor is at a concentration of about 3 μM.

5. The method of claim 1, wherein the BMP agonist is BMP4.

6. The method of claim 1, wherein the BMP agonist is at a concentration ranging from about 5 ng/ml to about 20 ng/ml.

7. The method of claim 1, wherein the BMP agonist is at a concentration of about 10 ng/ml.

8. The method of claim 1, wherein the at least one FGF agonist comprises FGF10 and keratinocyte growth factor (KGF).

9. The method of claim 1, wherein the at least one FGF agonist is at a concentration ranging from about 5 ng/ml to about 20 ng/ml.

10. The method of claim 1, wherein the at least one FGF agonist is at a concentration of about 10 ng/ml.

11. The method of claim 1, wherein retinoic acid is at a concentration ranging from about 20 nM to about 80 nM.

12. The method of claim 1, wherein retinoic acid is at a concentration of about 50 nM.

13. The method of claim 1, wherein the BMP agonist is BMP4, the at least one FGF agonist is KGF and FGF10, and wherein KGF, FGF10, and/or BMP4 are at a concentration of about 10 ng/ml.

14. The method of claim 1, wherein the inhibitor of ROCK is Y27632.

15. The method of claim 1, wherein the inhibitor of ROCK is at a concentration ranging from about 5 μM to about 15 μM.

16. The method of claim 1, wherein the inhibitor of ROCK is at a concentration ranging from about 5 μM to about 10 μM.

17. The method of claim 1, wherein the inhibitor of ROCK is at a concentration of about 10 μM in the first cell culture medium, and wherein the inhibitor of ROCK is at a concentration of about 5 μM in the second cell culture medium.

18. The method of claim 1, wherein insulin is at a concentration ranging from about 1 μg/ml to about 10 μg/ml.

19. The method of claim 1, wherein insulin is at a concentration of about 5 μg/ml.

20. The method of claim 1, wherein the EGF agonist is EGF.

21. The method of claim 1, wherein the EGF agonist is at a concentration ranging from about 0.05 ng/ml to about 0.5 ng/ml.

22. The method of claim 1, wherein the EGF agonist is at a concentration of about 0.1 ng/ml.

23. The method of claim 1, wherein the corticosteroid is hydrocortisone.

24. The method of claim 1, wherein the corticosteroid is at a concentration ranging from about 10 ng/ml to about 50 ng/ml.

25. The method of claim 1, wherein the corticosteroid is at a concentration of about 25 ng/ml.

26. The method of claim 1, wherein the cAMP pathway activator is cholera toxin.

27. The method of claim 1, wherein the cAMP pathway activator is at a concentration ranging from about 1 ng/ml to about 10 ng/ml.

28. The method of claim 1, wherein the cAMP pathway activator is at a concentration of about 8 ng/ml.

29. The method of claim 1, wherein the first culture medium or the second culture medium is serum-free.

30. The method of claim 1, wherein the first culture medium or the second culture medium comprises a serum substitute.

31. The method of claim 1, wherein the first culture medium or the second culture medium comprises a cell culture medium conditioned by feeder cells.

32. The method of claim 31, wherein the feeder cells are fibroblasts.

33. The method of claim 32, wherein the fibroblasts are 3T3-J2 cells.

34. The method of claim 1, wherein the first biomolecule or the second biomolecule comprises a solubilized basement membrane preparation from the Engelbreth-Holm-Swarm (EHS) mouse sarcoma.

35. The method of claim 1, wherein the first biomolecule or the second biomolecule comprises gelatin and/or collagen.

36. The method of claim 1, wherein the first cell culture substrate and the second cell culture substrate are identical, and wherein the first biomolecule and the second biomolecule are identical.

37. The method of claim 1, wherein the LBO comprises (i) lung epithelial cells expressing FOXA2, FOXA1, NKX2.1 and EPCAM, and (ii) mesenchymal progenitors expressing PDGFRa, CD90, TBX4 and HOXA5.

38. The method of claim 1, wherein in step (c) the LBO is dissociated to single cells.

39. The method of claim 1, wherein the mammalian pluripotent stem cells (PSCs) are human pluripotent stem cells (hPSCs).

40. The method of claim 1, wherein the mammalian pluripotent stem cells (PSCs) are embryonic stem cells (ESCs) and/or induced pluripotent stem cells (iPSCs).

41. The method of claim 1, wherein the lung progenitor cells comprise secretory-like cells, and airway basal-like cells.

42. The method of claim 41, wherein the lung progenitor cells further comprise fibroblasts, T cells and dendritic cells.

43. The method of claim 1, wherein the lung progenitor cells comprise cells expressing MUC1, NOTCH3, UPK3A, KRT4, and KRT13, and cells expressing p63, KRT5, KRT17, ITGB4, JAG2 and DLK2.

44. The method of claim 1, wherein the lung progenitor cells comprise MUC1^{hi}CD104^{lo} cells and MUC1^{lo}CD104^{hi} cells.

45. Lung progenitor cells generated by the method of claim 1.

46. The lung progenitor cells of claim 45, comprising secretory-like cells, and airway basal-like cells.

47. The lung progenitor cells of claim 46, further comprising fibroblasts, T cells and dendritic cells.

48. The lung progenitor cells of claim 45, comprising cells expressing MUC1, NOTCH3, UPK3A, KRT4, and KRT13, and cells expressing p63, KRT5, KRT17, ITGB4, JAG2 and DLK2.

49. A cell population comprising lung progenitor cells generated by the method of claim 1.

50. Lung progenitor cells generated in vitro, comprising secretory-like cells, and airway basal-like cells.

51. The lung progenitor cells of claim 50, further comprising fibroblasts, T cells and dendritic cells.

52. Lung progenitor cells generated in vitro, comprising cells expressing MUC1, NOTCH3, UPK3A, KRT4, and KRT13, and cells expressing p63, KRT5, KRT17, ITGB4, JAG2 and DLK2.

53. A cell population comprising the lung progenitor cells of claim 45.

54. A pharmaceutical composition comprising the lung progenitor cells of claim 45.

55. A method of treating a pulmonary disorder or injury in a subject in need thereof, the method comprising administering to the subject an effective amount of the lung progenitor cells of claim 45.

56. The method of claim 55, wherein the pulmonary disorder or injury is selected from the group consisting of: cystic fibrosis; emphysema; chronic obstructive pulmonary disease (COPD); pulmonary fibrosis; idiopathic pulmonary fibrosis (IPF); Hermansky-Pudlak Syndrome; hypersensitivity pneumonitis; sarcoidosis; asbestosis; autoimmune-mediated interstitial lung disease; pulmonary hypertension; lung cancer; acute lung injury (adult respiratory distress syndrome); respiratory distress syndrome of prematurity, chronic lung disease of prematurity (bronchopulmonary dysplasia); surfactant protein B deficiency, surfactant protein C deficiency, ABCA3 deficiency; NKX2.1 mutation; ciliopathies; congenital diaphragmatic hernia; pulmonary alveolar proteinosis; pulmonary hypoplasia; lung injury, and combinations thereof.

57. The method of claim 55, wherein the pulmonary disorder or injury is an interstitial lung disease or a congenital surfactant deficiency.

58. The method of claim 55, wherein the lung progenitor cells are non-syngeneic with the subject.

59. The method of claim 55, wherein the lung progenitor cells are syngeneic with the subject.

60. The method of claim 55, wherein the lung progenitor cells are allogeneic or xenogeneic with the subject.
